(19) United States
(12) Patent Application Publication
(10) Pub. No.: US 2009/0291091 A1
(43) Pub. Date: Nov. 26, 2009

(54) USE OF THE GLOBULAR DOMAIN OF ACRP30 FOR THE PREPARATION OF A MEDICAMENT FOR THE PREVENTION AND/OR TREATMENT OF THROMBOSIS-RELATED DISEASES

(75) Inventors: Bernard Bihain, Cancale (FR); Michel Dreano, Collonges-sous-Saleve (FR); Jennifer Hantson, Collonges-sous-Saleve (FR); Virginie Ogier, Nancy (FR); Pierre-Alan Vitte, Cranves-Sales (FR); Frances Yen-Potin, Ludres (FR)

Correspondence Address: SALIWANCHIK LLOYD & SALIWANCHIK;A PROFESSIONAL ASSOCIATION, PO Box 142950, GAINESVILLE, FL, 32614, US

(73) Assignee: Laboratoires Serono SA, Coinsins. Vaud (CH)

(21) Appl. No.: 11/997,181

(22) PCT Filed: Jun. 20, 2006
(86) PCT No.: PCT/EP2006/063341

§ 371 Date: Jan. 29, 2008

Related U.S. Application Data
(60) Provisional application No. 60/704,254, filed on Aug. 1, 2005.

(30) Foreign Application Priority Data
Jul. 29, 2005 (EP) 05107038.1

(51) Int. Cl. A61K 39/395; A61K 38/22

(52) U.S. Cl. 424/178.1; 514/12

(57) ABSTRACT

It is the object of the present invention to provide novel means for the treatment and/or prevention of thrombosis, tumor implantation, tumor seeding and metastasis. More specifically, the present invention relates to the use of a polypeptide comprising the globular head of Acrp30 for the manufacture of a medicament for treatment and/or prevention of thrombosis-related disorder, an hypertensive disorder of the pregnancy, tumor implantation, tumor seeding and metastasis.

### FIELD OF THE INVENTION

The present invention is generally in the field of thrombosis and of cancer. More specifically, the present invention relates to the use of a polypeptide comprising the globular head of Acrp30 for the manufacture of a medicament for treatment and/or prevention of a thrombosis-related disorder, an hypertensive disorder of the pregnancy, tumor implantation, tumor seeding and metastasis.

### BACKGROUND OF THE INVENTION

1. Acrp30

Adipose tissue, while long known for its capacity to store fat, has an important role as the source for a number of hormones and paracrine mediators, including resistin, adipsin, leptin, and TNF-α. Collectively, these molecules are termed adipokines, to emphasize their role as hormone and site of synthesis. Acrp30, also referred to as Adiponectin or ApM-1, is one such adipokine and is produced by adipose tissue. However, Acrp30 cannot be considered as an hormone because its concentration in plasma is not within the hormonal range. Indeed, Acrp30 concentrations in plasma vary from 2 to 18 μg/ml, whereas hormone concentrations are typically below or within the ng/ml range.

Mouse Acrp30 was first identified in 1995 (Scherer et al., 1995), and was shown to be up-regulated over 100-fold during adipocyte differentiation. The human homolog was identified in 1996 (Maeda et al., 1996). Acrp30 contains an amino-terminal signal sequence, followed by a central region comprising collagen repeats, and a carboxyl-terminal domain with homology to the globular complement factor Clq. This latter domain is commonly referred to as the “globular head” of Acrp30, Several studies have specifically focused on fragments of Acpr30 comprising the globular head (e.g., WO 01/51645 and Fruebis et al., 2001).

Different species of Acrp30 polypeptides, having different molecular weights, exist. The structure of these species of different apparent molecular weight was investigated (Tsao et al., 2002; Tsao et al., 2003). When expressed in bacteria as a full-length fusion protein and separated by gel-filtration chromatography, three species of Acrp30 were identified: hexamers and two species of trimers. Eukaryotic cell expression studies generated three Acrp30 species: a high-molecular weight (HMW) species, which is not seen in bacterially produced protein, and species corresponding to hexamers and one species of trimers.

Studies have demonstrated that Acrp30 is linked to obesity and type II diabetes. Genetic data have demonstrated linkage of type II diabetes with non-coding Single Nucleotide Polymorphisms (SNPs) located within the Acrp30 gene in a Japanese cohort of patients (Hara et al., 2002). It was further demonstrated that missense mutations affecting the globular head are correlated with serum levels of Acrp30 (Kondo et al., 2002).

In addition, serum levels of Acrp30 are decreased in several models of obesity, including leptin-deficient mice, leptin-receptor deficient mice, and monkey models (Hu et al., 1996; Yamauchi et al., 2001). In human studies, Acrp30 levels are inversely correlated to both diabetes and obesity, and they are further reduced in patients with coronary artery disorder (Arita et al., 1999). Further evidence for a causal relationship between reduced levels of Acrp30 and development of insulin resistance and type II diabetes was obtained by Lindsay et al., who showed that individuals in the Pima Indian population who had lower serum levels of Acrp30 were more likely to develop type II diabetes than those with higher levels (Lindsay et al., 2002). In 2002, it was found that homozygous Acrp30-deficient mice were not hyperglycemic when maintained on a normal diet, but they exhibited reduced clearance of serum free fatty acid. When switched to a high-fat, high-sucrose diet, they exhibited severe insulin resistance and demonstrated increased weight gain relative to control animals (Maeda et al., 2002).

In addition to its pivotal role in obesity and diabetes, Acrp30 has been suggested to play a role in other disorders. Specifically, association of serum or plasma levels of Acrp30 with polycystic ovary syndrome (Panidis et al., 2003), endometrial cancer (Petridou et al., 2003), preeclampsia (Ramsay et al., 2003) and the nephritic syndrome (Zoccali et al., 2003) has been observed. Acrp30 has also been shown to display anti-inflammatory properties (Yokota et al., 2000) and to alleviate fatty liver diseases in mice (Xu et al., 2003).

In addition, Clark et al. (2004) teaches that full-length Acpr30 down-regulates the production of TNF-alpha from myocardium. Clark et al. (2004) further discloses the use of full-length Acpr30 for the treatment of acute and chronic heart failure associated with myocardial ischemia.

2. Diseases Associated with Hypercoagulation and/or Hyper Platelet Aggregation.

Thromboembolic diseases are the third most common acute cardiovascular diseases, second to cardiac ischemic syndromes and stroke.

Thromboembolic diseases are caused by hypercoagulability or obstruction, which leads to the formation of thrombus in the deep veins of the legs, pelvis, or arms. As the clot propagates, proximal extension occurs, which may dislodge or fragment and embolize to the pulmonary arteries. This causes pulmonary artery obstruction and the release of vasoactive agents (ie, serotonin) by platelets increases pulmonary vascular resistance. The arterial obstruction increases alveolar dead space and leads to redistribution of blood flow, thus impairing gas exchange due to the creation of low ventilation-perfusion areas within the lung. Stimulation of irritant receptors causes alveolar hyperventilation. Reflex bronchoconstriction occurs and augments airway resistance. Lung edema decreases pulmonary compliance. The increased pulmonary vascular resistance causes an increase in right ventricular after load, and tension rises in the right ventricular wall, which may lead to dilatation, dysfunction, and ischemia of the right ventricle. Right heart failure can occur and lead to cardiogenic shock and even death. In the presence of a patent foramen ovale or atrial septal defect, paradoxical embolism may occur, as well as right-to-left shunting of blood with severe hypoxemia.

Currently available therapies of thromboembolitic diseases include treatments using an anti-coagulant agent, treatments using a fibrinolytic agent and surgery (Nutescu, 2004; Haines, 2004; Hawkins, 2004). Most currently available therapies for the treatment of thromboembolitic diseases are based on anti-coagulant properties of an agent, said agent degrading the protein component of a blood clot. Thus far hirudin, which is both an anti-coagulant and anti-aggregant, is the sole agent acting directly on platelets.

2.1. Venous Thrombosis-Related Diseases

Venous thromboembolism, the syndrome in which blood clots (thrombi) form in the deep veins and often break loose to travel to the lungs, is one of the most difficult and serious problems in modern medicine. Venous thromboembolism encompasses two interrelated conditions that are part of the same spectrum, deep venous thrombosis (DVT) and pulmonary embolism (PE). PE is the obstruction of blood flow to one or more arteries of the lung by a blood clot lodged in a pulmonary vessel. PE and DVT can occur in the setting of disease processes, following hospitalization for serious illness, or following major surgery.

Both DVT and PE frequently remain undiagnosed because they may be clinically unsuspected. The spectrum of disease ranges from clinically unsuspected, to clinically unimportant, to massive embolism causing death. Untreated acute proximal DVT causes clinical PE in 33-50% of patients. Untreated PE often is recurrent over days to weeks and can either improve spontaneously or cause death. About one third of PE cases are fatal. 67% of these are not diagnosed pre-mortem, and 34% occur rapidly. A high rate of clinically unsuspected DVT and PE leads to significant diagnostic and therapeutic delays, and this accounts for substantial morbidity and mortality.

Anti-coagulant agents prevent the formation of blood clots, and have been the mainstay of therapy for DVT and PE since the initial introduction of heparin into clinical use in the 1930s. Anti-coagulant drugs currently on the market for treating thromboembolitic diseases include intravenous heparin, which acts by inactivating thrombin and several other clotting factors required for a clot to form, and oral anti-coagulants such as warfarin and dicumarol, which act by inhibiting the liver's production of vitamin K-dependent factors crucial to clotting. The mechanism of action of anti-vitamin K agents is to reduce availability of vitamin K in the liver. Therefore, warfarin and dicumarol take days to weeks to be effective. Both heparin and anti-vitamin K agents act on the coagulation system, which involves the activation of a cascade of proteolytic enzyme present in the plasma. Both heparin and anti-vitamin K agents primarily act on the activity of the proteolytic enzymes of the activation cascade. This activation cascade ultimately produces thrombin, which cleaves fibrinogen in such a way to produce fibrin, the proteic part of blood clot. Platelets constitute the cellular part of the plot. Aggregation is a process through which platelets, activated by substances such as, e.g., thrombin, bind to one another to form the cellular component of the clot.

Fibrinolytic therapy allows removing an abnormal clot by activating a plasma proenzyme, plasminogen, to its active form, plasmin. Plasmin degrades fibrin to soluble peptides. Besides restoring an outflow channel, lysis of a thrombus has been demonstrated to preserve and restore normal venous valve structure and function if performed early enough in the course of the disease process. Marketed drugs that belong to this category include streptokinase and urokinase.

Unfortunately, when thrombosis is extensive, fibrinolysis alone may be inadequate to dissolve the volume of thrombus present, and surgery becomes mandatory. Venous thrombectomy, although rarely used, may improve the long-term outcome.

2.2. Arterial Thrombosis-Related Diseases

The central importance of platelets in the development of arterial thrombosis and cardiovascular diseases is well established (Jackson and Schoenwaelder, 2003; Bhatt and Topol, 2003). No other single cell type is responsible for as much morbidity and mortality as the platelet and, as a consequence, it represents a major target for therapeutic intervention. Various anti-aggregant therapies have proved successful in the treatment of arterial thrombosis and cardiovascular diseases. For example, the clinical data supporting the efficacy of aspirin, an inhibitor of the thromboxane pathway, in atherosclerosis are overwhelming. The Antiplatelet Trialists' Collaboration (ATC) found an approximately 25% relative risk reduction of vascular death, myocardial infarction or stroke for antiplatelet therapy, primarily aspirin, versus placebo (ATC, 1994). Clopidogrel and Ticlopidine, which are irreversible platelet inhibitors, have also been proven to be efficient therapies for the treatment of arterial thrombosis. Indeed, there is a large body of data supporting the efficacy of ticlopidine in conditions such as claudication, unstable angina, coronary artery bypass surgery, peripheral artery bypass surgery and cerebrovascular disease.

2.3. Hypertensive Disorders of the Pregnancy

Platelet activation is also an important aspect of the pathogenesis of hypertensive disorders of the pregnancy and its complications. Hypertensive disorders occur in 6% to 8% of all pregnancies, and are the second leading cause of maternal death, and contribute to significant neonatal morbidity and mortality. In such disorders, platelet activation occurs as a result of the widespread endothelial dysfunction that is associated with this disorder. Indeed, antiplatelets drugs are effective in preventing the complications associated with hypertensive disorders of the pregnancy, as well as preventing the occurrence of the disorder to a certain extent (Nadar and Lip, 2004).

2.4. Cancer

In patients with venous thromboembolism, there is a concomitant cancer in 15 to 20% of patients. It was observed that anti-coagulant drugs used in the treatment of thrombosis were also beneficial for the treatment of cancer. This was first observed by Michaels, who found that oral anti-coagulants reduced mortality in cancer patients (Michaels., 1964). Berkarda et al. showed that the warfarin anti-coagulant inhibited metastasis formation in mice inoculated with Lewis lung tumor (Berkarda et al., 1978). The observation was later confirmed in human (Zacharski et al., 1990; Berkarda et al., 1992). Recently, Hu et al. have shown that hirudin, a potent inhibitor of thrombin, inhibits tumor implantation, seeding and spontaneous metastasis (Hu et al., 2004).

Anti-coagulants and/or anti-aggregants are thus efficient not only for the treatment of arterial thrombosis and venous thrombosis, but also for the prevention of metastasis formation in cancer, and for the treatment of complications associated with hypertensive disorders of the pregnancy.

### SUMMARY OF THE INVENTION

It has been found in the frame of the present invention that fragments of Acrp30 comprising the globular head exhibit anti-coagulant and/or anti-aggregant properties. In addition, two novel naturally-occurring cleavage products of Acrp30 of 15.4 kDa and of 20 kDa have been identified.

Therefore, a first aspect of the invention relates to the use of an Acrp30g polypeptide, or of an agonist thereof, for the manufacture of a medicament for the treatment and/or the prevention of thrombosis.

A second aspect relates to the use of an Acrp30g polypeptide or of an agonist thereof, for the manufacture of a medicament for the treatment and/or the prevention of tumor implantation, tumor seeding and metastasis.

A third aspect relates to the use of an Acrp30g polypeptide or of an agonist thereof, for the manufacture of a medicament for the treatment and/or the prevention of hypertensive disorders of the pregnancy.

A fourth aspect relates to the use of a nucleic acid molecule for manufacture of a medicament for the treatment and/or prevention of a disease selected from the group consisting of a thrombosis-related disease, an hypertensive disorder of the pregnancy, tumor implantation, tumor seeding and metastasis, wherein the nucleic acid molecule comprises a nucleic acid sequence encoding an Acrp30g polypeptide.

A fifth aspect relates to the use of a vector for inducing and/or enhancing the endogenous production of an Acrp30g polypeptide, or of an agonist thereof, in a cell in the manufacture of a medicament for the treatment and/or prevention of a disease selected from the group consisting of a thrombosis-related disease, an hypertensive disorder of the pregnancy, tumor implantation, tumor seeding and metastasis.

A sixth aspect relates to the use of a cell that has been genetically modified to produce an Acrp30g polypeptide, or of an agonist thereof, in the manufacture of a medicament for the treatment and/or prevention of a disease selected from the group consisting of a thrombosis-related disease, an hypertensive disorder of the pregnancy, tumor implantation, tumor seeding and metastasis.

A seventh aspect relates to a method for treating and/or preventing a disease selected from the group consisting of a thrombosis-related disease, an hypertensive disorder of the pregnancy, tumor implantation, tumor seeding and metastasis comprising administering to a patient in need thereof an effective amount of an Acrp30g polypeptide or of an agonist thereof, optionally together with a pharmaceutically acceptable carrier.

An eighth aspect relates to an antibody specifically binding to an Acrp30 fragment characterized by a mass of about 15.4 kDa and/or about 20 kDa.

A ninth aspect relates to diagnostic kits comprising such antibodies in accordance with the invention.

An tenth aspect relates to methods of diagnosing a disease selected from the group consisting of a thrombosis-related disease, an hypertensive disorder of the pregnancy, a metabolic disease, tumor implantation, tumor seeding and metastasis, in which either the presence or the absence, or the levels, of an Acrp30 fragment characterized by a mass of about 15.4 kDa and/or 20 kDa is assessed in a plasma sample.

A eleventh aspect relates to the use of an Acrp30g polypeptide for the manufacture of a medicament for the treatment and/or the prevention of a metabolic disorder characterized in that the Acrp30g polypeptide comprises a fragment of Acrp30 of about 20 kDa.

### BRIEF DESCRIPTION OF THE DRAWINGS

FIG. 1 shows the effect of Acrp30g-1 and of Acrp30g-2 on the volume of blood collected either by retroorbital puncture (ROP) or by decapitation in db/db mice.

FIG. 2 demonstrates the anti-aggregant and/or anti-coagulant activity of an acute subcutaneous treatment with Acrp30g-2 in C57BL/6 mice. Mice were injected with 100 μg/kg of Acrp30g-2 (+). The controls mice (−) did not received any injection. The Howell time and the platelet rich plasma (PRP) were calculated as described in Example 2. A This Figure shows the effect of Acrp30g-2 injection on the Howell time. The white bar corresponds to the negative control. The gray bars correspond to the mice injected with Acrp30g-2. B. This Figure shows the effect of Acrp30g-2 injection on the PRP. The white bar corresponds to the negative control. The gray bars correspond to the mice injected with Acrp30g-2. C. This Figure shows the effect of increasing doses of Acrp30g-2 injection on the Howell time.

FIG. 3 shows the effect of an acute subcutaneous treatment with Acrp30g-2 on the Howell time in C57BL/6 mice. Mice were injected with a slaine solution, Acrp30g-2 or heparin.

FIG. 4 shows the effect of a 2 weeks treatment with Acrp30g-2 on tail bleeding time in C57BL/6 mice. The mice were either fed with high-fat diet (obese) or normal diet (lean). Mice were injected with 100 μg/kg of Acrp30g-2 (+) or with a saline solution (−).

FIG. 5 shows the lack of significant effect of a 2 weeks treatment with Acrp30g-2 on: A thrombin clotting time (TCT); B. platelet number (PLT); and C. fibrinogen levels. Mice were injected with Acrp30g-2 (+) or with a saline solution (−).

FIG. 6 shows the effect of a single subcutaneous injection comprising increasing doses of Acrp30g-2 on tail bleeding time in C57BL/6 mice. “N” indicates the number of mice tested for each dose.

FIG. 7 shows the effect, on in vitro clotting time, of supplementing platelet rich plasma from mildly obese mice with Acrp30g-2 (+). The controls correspond to platelet rich plasma from mildly obese mice supplemented with saline solution (−). After addition of Acrp30g-2, the samples were supplemented either with 22 mM Ca²⁺ or with 11 mM Ca²⁺.

FIG. 8 shows the survival rate of female C57BL/6 mice suffering from acute pulmonary embolism after tail vein injection of collagen (−). The effect of an injection of 500 μg/kg of Acrp30g-2 is also shown (+).

FIG. 9 shows the affect of Acrp30g-2 on survival rate and ECG profile in pulmonary embolism mouse model. A Typical ECG profile before, 30, 60 and 90 sec after tail vein injection of 375 μg/kg collagen and 45 μg/kg epinephrine into mice anesthetized with sodium pentobarbital. The 6 distal derivations, I, II, III, aVF, avR and avL are indicated. B. Four groups of 12 mice were injected IV with saline (□), 100 (▪), 130 (▴) or 200 (Δ) μg/kg Acrp30g-2, followed 5 min after the collagen/epinephrine challenge as described in a. Survival rate is indicated as the number of survivors at each time point. C. Derivation II of the ECG profile of a survivor 30 and 60 sec after injection of 200 μg/kg Acrp30g-2. The typical biphasic peak was not observed during the duration of the experiment.

FIG. 10 shows the effect of Acrp30g-2 injection on the survival rate of female C57BL/6 mice suffering acute pulmonary embolism after tail vein injection of collagen. Acrp30g-2 was injected three hours prior to collagen challenge.

FIG. 11 shows the effect of Acrp30g-2 injection on the survival rate of female C57BL/6 mice suffering acute pulmonary embolism after tail vein injection of collagen. 200 μg/kg of Acrp30g-2 or an equivalent volume of saline solution was injected intravenously 30 seconds after collagen injection.

FIG. 12 shows the effect of Acrp30g-2 as preventative or curative measure for pulmonary embolism in mouse. A Five groups of mice were injected with saline (n=20, □), 400 μg/kg Acrp30g-2 (n=12, □), 125 IU/kg (n=12, ▴) or 500 IU/kg heparin (n=12, Δ), or both 400 μg/kg Acrp30g-2 and 500 IU/kg heparin (n=8, ♦). Heparin and Acrp30g-2 were administered IP 30 min and IV 5 min, respectively, before the collagen and epinephrine challenge. Survival rate was monitored and is indicated on the left panel. Statistical testing of low dose efficacy by Kaplan-Meier analysis is shown on the right panel. B. Kaplan-Meyer analysis of the data shown in FIG. 11A. C. In three groups of 12 mice, either saline (□), 100 (▪), or 200 (▴) μg/kg Acrp30g-2 was administered IV 60 sec after the collagen/epinephrine challenge. Results are indicated as % survival rate over time. Statistical differences were tested using χ² method. D. Mice were injected SC with the indicated concentrations of Acrp30g-2 (n=10 for each group) 3 h before the collagen/epinephrine challenge.

FIG. 13 shows the effect of Acrp30g-2 on thrombin proteolytic activity. Thrombin proteolytic activity was measured as fibrin formation from fibrinogen induced by thrombin in the absence or presence the indicated concentrations of Acrp30g-2 or heparin. Results are indicated as the mean±SEM of triplicate determinations.

FIG. 14 shows the effect of Acrp30g-2 on platelet aggregation. A Platelet aggregation induced by 10 μg/mL collagen and 1 μg/mL (5.5 μM) epinephrine was measured in mouse platelet rich plasma (PRP) in the absence or in the presence of 400 or 800 ng/ml Acrp30g-2. Results are expressed as the average % platelet aggregation observed between 2 and 4 min as compared to control. B-C. Human platelet aggregation induced by 10 μg/mL collagen and 1 μg/mL epinephrine, 10 μM ADP (d) was measured in the absence (□) or presence (▪) of 800 ng/ml Acrp30g-2. D. Washed Human platelet aggregation induced by 0.1 U/mL thrombin was measured in the absence (□) or presence (▪) of 800 ng/ml Acrp30g-2 using washed platelets.

FIG. 15 shows the absence of effect of full-length Acrp30 on thrombin-induced platelet aggregation.

FIG. 16 shows the effect of Acrp30g-2 on platelet aggregation induced by different doses of thrombin. A 0.1 U/ml of thrombin was injected. B. 0.5 U/ml of thrombin was injected.

FIG. 17 shows the effect of Acrp30g-2 on thrombin-induced platelet aggregation when Acrp30g-2 is added before thrombin.

FIG. 18 shows the effect of Acrp30g-2 on thrombin-induced platelet aggregation when Acrp30g-2 is added after thrombin.

FIG. 19 compares the effect of Acrp30g-2, heparin and aspirin on thrombin-induced platelet aggregation.

FIG. 20 shows the identification of 15 kDa globular head of Acrp30 in human plasma. A Western blot of eluted fraction of immunoprecipitation (IP) on fresh human plasma using a novel conformation-dependent affinity purified antibody directed against the globular head of human Acrp30. The Western blot was revealed by antibodies directed against the globular head of Acrp30 (lane 1) or by antibodies directed against the collagen tail of Acrp30 (lane 2) as described in detail in Example 18. Western blot on human plasma was revealed by antibodies directed against the globular head of Acrp30 (lane 3) or antibodies directed against the collagen tail of Acrp30 (lane 4). B. Molecular mass determination of recombinant Acrp30g-2 by gel filtration on Superdex 200 HR10-30. Chromatography was performed at 0.5 mL/min in a Superdex 200 HR10-30 column (GE-Healthcare) with PBS buffer (30 mM Sodium Phosphate, 150 mM NaCl, pH 7.4) (see Example 18). Molecular mass standards cytochrome c, myoglobin, carbonic anhydrase and bovine serum albumin are represented () by a number from 1 to 4, respectively. Data for Acrp30g-2 (◯) is also indicated by an arrow. A chromatogram of Acrp30g-2 is represented in Insert. C. Western blot of eluted fraction of IP performed on fresh human plasma of 2 subjects: a male (lane 1) and a female (lanes 3 and 5) or on recombinant Acrp30g-2 (lanes 4 and 6). Recombinant Acrp30g-2 was also loaded directly on the gel without IP (lane 2). The Western blot was revealed by antibody directed against the globular head of Acrp30 (lanes 1 to 4) or by antibody directed against the collagen tail of Acrp30 (lanes 5 and 6). D. Surface-enhanced laser desorption ionization time-of-flight mass spectrometry profiles (14500-18000 mass-to-charge ratio) obtained by spiking recombinant Acrp30g-2 in human all blood before and after coagulation. Affinity purified anti-Acrp30g-2 antibodies were covalently immobilized on Protein Chip Arrays and incubated with human all blood containing 10 μg/mL of recombinant Acrp30g-2 (see Example 18). (A) 10 μg/mL recombinant Acrp30g-2 was spiked in fresh human blood and coagulation was prevented to occur by EDTA. (B) 10 μg/mL recombinant Acrp30g-2 was spiked in fresh human blood and coagulation was allowed to occur for 30 min at 37° C. (C) 10 μg/mL recombinant Acrp30g-2 was spiked in fresh human all blood after coagulation has occurred i.e. serum. The determined m/z ratio of recombinant Acrp30g-2 is also indicated.

FIG. 21 shows the effect of Acrp30g-2 after induction of PE in eNOS−/− mice and in C57BI6/J mice, pre-treated or not with the eNOS inhibitor Nω-Nitro-L-arginine methyl ester hydrochloride (L-NAME). A A saline solution (n=6) or Acrp30g-2 (300 μg/kg, n=6 per group) was injected intravenously (IV) 5 min before induction of PE (t=0 min) by injection of collagen (375 μg/kg) and epinephrine (45 μg/kg) in eNOS−/− mice. Results are expressed as the average survival time after induction of PE. B. One group of C57BI6/J mice was injected with a saline solution (n=9) and two other groups with 300 μg/kg of Acrp30g-2 (n=9 and 10) intravenously 5 min before the collagen/epinephrine challenge was performed as in FIG. 2 (t=0 min). In one group of mice that received Acrp30g-2 (n=9), L-NAME (100 mg/kg) was also injected IP 1 h before induction of PE. Results are expressed as the % survival rate at t=10 min after induction of PE.

FIG. 22 shows the effect of Acrp30g-2 on a mouse arterial thrombus model. The carotid arteries of anesthetized C57BI/6J mice were exposed and blood flow was monitored with a Transonic® flowprobe throughout the experiment. After the rate was stabilized (basal), thrombosis was induced by applying filter paper saturated with 3.75% FeCl₃. When a 50% decrease in blood flow was achieved, a physiological saline solution (□, n=5) or 400 μg/kg of Acrp30g-2 (▪, n=6) was injected. In a third group of mice, 100 mg/kg L-NAME (▴, n=3) was injected IP 1 h before induction of arterial thrombosis. Results are expressed as mean blood flow observed before, during, and after application of saline, or Acrp30g-2 L-NAME. The carotid arteries were exposed to FeCl₃ during the entire experiment.

FIG. 23 compares the effect of Acrp30g-2 and of full-length Acrp30 (fl-Acrp30) on platelet aggregation. Collagen and epinephrine-induced platelet aggregation was measured in human PRP in the absence (n=4) or the presence of 400 ng/ml of Acrp30g-2 (n=6) or 800 ng/ml of full-length Acrp30 (n=3). In another group (n=6), L-NAME was preincubated with PRP 10 min before addition of Acrp30g-2, collagen and epinephrine. Results are expressed as the average % platelet aggregation 5 min after addition of collagen/epinephrine.

FIG. 24 shows the effect of Acrp30g-2 on NO production in ECV 304 cells. A. ECV 304 cells were incubated 5 min at 37° C. in the presence of increasing concentrations of Acrp30g-2 or Acrp30 (fl-Acrp30). After which the cell media was recovered and the concentration of nitrate and nitrite determined using a fluorimetric assay. Results are expressed as mean % of the control. B. ECV 304 cells were preincubated 30 min at 37° C. in PBS containing 0.2% BSA and increasing concentrations of L-NAME. Acrp30g-2 (50 ng/ml) was then added followed by incubation for 2 h at 37° C. Nitrate and nitrite levels in the cell media was then determined. Results are expressed as mean % of the value obtained in absence of L-NAME.

### BRIEF DESCRIPTION OF THE SEQUENCES

SEQ ID NO: 1 corresponds to the amino acid sequence of the full length Acrp30 polypeptide.

SEQ ID NO: 2 corresponds to an Acrp30 polypeptide referred to as Acrp30g-1.

SEQ ID NO: 3 corresponds to an Acrp30 polypeptide referred to as Acrp30g-2.

### DETAILED DESCRIPTION OF THE INVENTION

In the frame of the present invention, it has been found that fragments of Acrp30 comprising the globular head inhibit thrombin-induced platelet aggregation, and exhibits a potent anti-aggregant activity.

Specifically, it has been shown that chronic treatments of normal or db/db mice with a polypeptide of SEQ ID NO: 2 or 3 (further referred to as Acrp30g-1 and Acrp30g-2 respectively) increased the blood volume recovered after bleeding (Example 1). It was further shown that acute treatment of normal mice or chronic treatment of db/db with Acrp30g-2 induced a significant increase of the Howell time without modifying the platelet number and without visible gastric prohemorrhagic effect (Examples 2 and 3). It was also demonstrated that chronic treatment of lean or obese mice with Acrp30g-2 increased the tail bleeding time, with no significant effect on thrombin clotting time, platelet number or circulating concentration of fibrinogen (Examples 4 and 5). It was further found that acute treatment of normal mice with Acrp30g-2 increased the tail bleeding time (Examples 6 and 7). A preventive treatment with Acrp30g-2 of a mouse model developing a collagen induced acute deep venous thromboembolism leading to a rapid pulmonary embolism and death allowed a significant reduction of death (Example 10). A curative treatment with Acrp30g-2 was shown to induce a significant reduction of death in this animal model (Example 11). Data show that Acrp30g-2, at 400 μg/kg, is more efficient that heparin, injected at a higher dose than the current therapeutic dose, for increasing the survival rate in a mouse model for pulmonary embolism (Example 12). In this mouse model, heparin and famoxin display a cumulative effect when injected simultaneously (Example 12). Acrp30g-2 inhibits platelet aggregation induced either by collagen or by thrombin, but does not inhibit aggregation induced by ADP (Example 15). This effect is not seen with full-length Acrp30, which does not inhibits platelet aggregation induced by thrombin (Example 16). It has further been demonstrated that Acrp30g-2 causes desaggregation of human platelet activated by thrombin, whereas neither heparin nor aspirin cause desaggregation of human platelet activated by thrombin (Example 17). It was also demonstrated that the nitric oxide synthase (eNOS) is critical for the anti-thrombotic effect of Acrp30g-2 (Example 26) and that Acrp30g-2 but not full-length Acrp30 increased NO production (Example 29). Finally, it was also shown that Acrp30g-2 restored arterial blood flow in a mouse model for arterial thrombosis (Example 27).

The experimental evidence presented herein therefore provides for a new possibility of treating a thrombosis-related disease, tumor implantation, tumor seeding, metastasis, as well as preventing the complications associated with hypertensive disorders of the pregnancy.

In addition, two novel naturally-occurring cleavage products of Acrp30 have been identified in the frame of the present invention (Example 19). The first cleavage products has a mass of about 15 kDa, and it has been shown that it corresponds to a naturally-occurring polypeptide of SEQ ID NO: 3. It has further been shown that it undergoes structural changes during coagulation (Example 21). The second cleavage product is 20 kDa, and it was shown that its presence in plasma is correlated with free fatty acid levels and resting energy expenditure in obese individuals (Example 20).

The experimental evidence presented herein therefore provides for a new possibility of diagnosing metabolic diseases, thrombosis-related diseases, tumor implantation, tumor seeding, metastasis and hypertensive disorders of the pregnancy using antibodies binding to an Acrp30 fragment characterized by a mass of about 15.4 kDa and/or of about 20 kDa.

In a first aspect, the invention therefore relates to the use of an Acrp30g polypeptide or of an agonist thereof for the manufacture of a medicament for the treatment and/or the prevention of a thrombosis-related disease.

The term “Acrp30 polypeptide”, as used herein, refers to a full-length or mature Acrp30 protein and to fragments thereof having biological activity.

The term “Acrp30g polypeptide”, as used herein, refers to a polypeptide comprising a fragment of Acrp30, said fragment (i) comprising amino acids 114 to 244 of SEQ ID NO: 1 and (ii) lacking amino acids 1 to 70 of SEQ ID NO: 1, wherein said polypeptide has biological activity. The term also encompasses muteins of such fragments of the Acrp30 protein. The term further encompasses homologues of a human Acrp30g polypeptide in other species. However, a human Acrp30g is preferably used in the methods and uses of the present invention. The Acrp30g polypeptide may correspond to a fused protein, a functional derivative, an active fraction or fragment, a circularly permutated derivative or a salt of a polypeptide comprising amino acids 114 to 244 of SEQ ID NO: 1 and lacking amino acids 1 to 70 of SEQ ID NO: 1, or a mutein thereof.

As used herein, the term “biological activity” of an Acrp30g polypeptide refers to anti-coagulant activity and/or anti-aggregant activity. The biological activity of an Acrp30g polypeptide can be assessed as described in any of the Examples. The anti-coagulant and/or anti-aggregant activity of an Acrp30g polypepitde can be assessed by measuring, e.g., the Howell time as described in Example 2, or by measuring the thrombin-induced platelet aggregation as described in Example 14.

In a preferred embodiment, an Acrp30g polypeptide has biological activity if the Howell time, preferably measured as described in Example 2, increases in a dose dependent manner upon injection of increasing doses of Acrp30g polypeptides. In a more preferred embodiment, an Acrp30g polypeptide has biological activity if the Howell time is increased of at least 5%, 10%, 15%, 20%, 25%, 30%, 40% or 50% when a dose of 0.3 mg/ml of Acrp30g is injected to a mouse as compared to the control (e.g., a mouse injected with a saline solution). In a most preferred embodiment, said Howell time is increased of at least 15% when a dose of 0.3 mg/ml of Acrp30g is injected to a mouse as compared to as compared to the control.

The term “agonist of an Acrp30g polypeptide” as used herein, relates to a molecule stimulating or imitating the anti-coagulant and/or anti-aggregant activity mediated by the Acrp30g polypeptide. Such agonists encompass any agent enhancing the biological activity of an Acrp30g polypeptide. All methods and uses disclosed herein may be carried out either with an Acrp30g polypeptide or with an agonist thereof.

The agonist of an Acrp30g polypeptide may be naturally occurring and synthetic compounds. Such compounds include, e.g., natural ligands, agonistic small molecules, agonistic antibodies and agonistic aptamers. As used herein, the term “natural ligand” refers to any signaling molecule that binds to an Acrp30g polypeptide in vivo and includes molecules such as, e.g., lipids, nucleotides, polynucleotides, amino acids, peptides, polypeptides, proteins, carbohydrates and inorganic molecules. As used herein, the term “small molecule” refers to an organic compound. As used herein, the term “antibody” refers to a protein produced by cells of the immune system or to a fragment thereof that binds to an antigen. As used herein, the term “aptamer” refers to an artificial nucleic acid ligand (Ellington and Szostak, 1990).

In a preferred embodiment, said agonist corresponds to a small molecule, an aptamer or an antibody that binds to a receptor for Acrp30, thereby activating said receptor. Preferably, said agonist corresponds to an agonistic antibody that binds to a receptor for Acrp30, Several receptors for Acrp30, which include T-cadherin (Hug et al., 2004 and WO 2005/057222), Omoxin (WO 03/013578), and AdipoRl and AdipoR2 (Yamauchi et al., 2003), are know in the art. Preferably, said agonist binds to T-cadherin or to adipoR1.

One example of a method that may be used for screening candidate compounds for an agonist is a method comprising the steps of:
a) contacting an Acrp30g polypeptide with the candidate compound; and
b) testing the activity of said Acrp30g polypeptide in the presence of said candidate compound;
   wherein an increased activity of said Acrp30g polypeptide in the presence of said compound compared to the activity of said Acrp30g polypeptide in the absence of said compound indicates that the compound is an agonist of said Acrp30g polypeptide. Preferably, such a method also comprises the step of testing the activity of said Acrp30g polypeptide in the absence of said candidate compound.

Another example of a method that may be used for screening candidate compounds for an agonist is a method comprising the steps of:
a) contacting a cell expressing a receptor for Acrp30 with the candidate compound; and
b) testing the activity of Acrp30g in the presence of said candidate compound;
   wherein an increased activity of said Acrp30g polypeptide in the presence of said compound compared to the activity of said Acrp30g polypeptide in the absence of said compound indicates that the compound is an agonist of said Acrp30g polypeptide. Preferably, such a method also comprises the step of testing the activity of said Acrp30g polypeptide in the absence of said candidate compound.

The terms “treating” and “preventing”, as used herein, should be understood as preventing, inhibiting, attenuating, ameliorating or reversing one or more symptoms or cause(s) of a disease selected from the group consisting of a thrombosis-related disease, an hypertensive disorder of the pregnancy, tumor implantation, tumor seeding and metastasis, as well as symptoms, diseases or complications accompanying said disease. When “treating” a disease selected from the group consisting of a thrombosis-related disease, an hypertensive disorder of the pregnancy, tumor implantation, tumor seeding and metastasis, the substances according to the invention are given after onset of the disease, “prevention” relates to administration of the substances before signs of disease can be noted in the patient.

As used herein, the term “thrombosis-related disease” encompasses both arterial thrombosis-related diseases, venous thrombosis-related diseases and diseases related thereto.

The terms “venous thrombosis-related disease” and “thromboembolic disease” are used interchangeably herein. These terms encompasses the following diseases, disorders and syndromes: thromboembolism, deep vein thrombosis (DVT), thrombophlebitis, venous claudication, venous thromboembolism or venous thromboembolism (VTE), pulmonary thromboembolism (PTE), pulmonary embolism (PE), venous thrombosis, deep vein thrombus, deep venous thrombus, obstructed venous outflow, chronic venous insufficiency (CVI), postphlebitic syndrome. These diseases include those described in detail in the “Background of the invention” and those disclosed in the “The Merck Manual for Diagnosis and Therapy”, Seventeenth Edition, published by Merck Research Laboratories, 1999. Preferably, said thrombosis-related disease is selected from the group consisting of deep vein thrombosis (DVT), pulmonary embolism (PE), chronic venous insufficiency (CVI), thrombophlebitis and postphlebitic syndrome. Most preferably, said thromboembolitic disease is DVT or PE.

The terms “arterial thrombosis” or “arterial thrombosis-related disease”, as used herein, encompasses the following diseases, disorders and syndromes: coronary arterial thrombosis (e.g., unstable angina, stable angina or myocardial infarction), ischemic stroke, intermittent claudication and atrial fibrillation. The arterial thrombosis may be associated with a primary and/or secondary ischemic event. For example, a coronary arterial thrombosis may be associated with a primary and/or secondary ischemic event selected from the group consisting of myocardial infarction, unstable or stable angina, acute reocclusion after percutaneous transluminal coronary angioplasty or restenosis. An ischemic stroke may be associated with, e.g., a primary and/or secondary ischemic event selected from the group consisting of a thrombotic stroke, a transient ischemic attack and a reversible ischemic neurological deficit. The Acrp30g polypeptide may either be used:
(i) during the acute phase of the arterial thrombosis;
(ii) during a chronic arterial thrombosis; and/or
(iii) to treat and/or to prevent a secondary ischemic event.

Other thrombosis-related diseases include, e.g.:
(i) Thrombosis of arterio-veinous shunts (e.g. surgical fistulas);
(ii) Diseases associated with increased clotting and thrombotic risk such as, e.g., disseminated intravascular coagulation, acquired or congenital hypercoagulation syndromes (e.g. anti-phospholipid syndrome, nephrotic syndrome), thrombophilia (e.g. primary thrombophilia, myeloproliferative syndrome); and
(iii) Diseases associated with micro-vessel partial or complete occlusion such as, e.g., thrombotic microangiopathie, diabetic micro and macro-angiopathies (proliferative and non proliferative), osteonecrosis, frost, Raynaud syndrome (and associated conditions such as, e.g., systemic scleroderma, Sharp syndrome and systemic lupus), erectile dysfunction, angiomas, angeitis.
(iv) Clotting and organ loss after organ reimplantation such as, e.g., finger reimplantation.

The capacity of an Acrp30g polypeptide or of an agonist thereof to prevent or to treat a thrombosis-related disease can for example be assessed as described in Example 10 and 11.

In a second aspect, the invention relates to the use of an Acrp30g polypeptide or of an agonist thereof for the manufacture of a medicament for the treatment and/or the prevention of tumor implantation, tumor seeding and metastasis.

As used herein, the term “tumor” refers to a malignant tumor. In particular, this term encompasses primary cancerous tumors and metastatic tumors. This term encompasses, e.g., colon cancer, endometrial cancer, breast cancer, melanomas, myelomas, sarcomas, lymphomas, leukemias such as chronic or acute lymphocytic leukemia, carcinomas such as non-small cell lung carcinoma and breast carcinoma.

The capacity of an Acrp30g polypeptide or of an agonist thereof to inhibit tumor implantation, tumor seeding and metastasis can be assessed as described in, e.g., Examples 23 to 25 and in Hu et al. (2004).

In a preferred embodiment, the tumor implantation, tumor seeding and/or metastasis is associated with a thrombosis-related disease. As a matter of fact, there is a concomitant cancer in a number of patients suffering from thrombosis-related diseases. Specifically, there is a concomitant cancer in 15 to 20% of patients suffering from venous thromboembolism.

In a third aspect, the invention further relates to the use of an Acrp30g polypeptide or of an agonist thereof for the manufacture of a medicament for the treatment and/or the prevention of a hypertensive disorder of the pregnancy.

As used herein, the term “hypertensive disorder of the pregnancy” encompasses gestational hypertension (GH), nonproteinuric gestational hypertension, preeclampsia, nonproteinuric preeclampsia, eclampsia, nonproteinuric eclampsia and pregnancy-induced hypertension (PIH).

In a preferred embodiment, the hypertensive disorder of the pregnancy is associated with a thrombosis-related disease. Indeed, antiplatelets drugs are effective in preventing the complications such as, e.g., thrombosis-related disorders, associated with hypertensive disorders of the pregnancy, as well as preventing the occurrence of the hypertensive disorder of the pregnancy to a certain extent (Nadar and Lip, 2004).

The invention further relates to methods of treating and/or preventing a disease selected from the group consisting of a thrombosis-related disease, an hypertensive disorder of the pregnancy, tumor implantation, tumor seeding and metastasis comprising the step of administering an Acrp30g polypeptide or an agonist thereof to an individual suffering from said disease.

In a preferred embodiment of the present invention, the Acrp30g polypeptide is selected from the group consisting of:
a) A polypeptide comprising amino acids 114 to 244 of SEQ ID NO: 1;
b) A polypeptide comprising SEQ ID NO: 2;
c) A polypeptide comprising SEQ ID NO: 3;
d) A polypeptide comprising amino acids 106 to 244 of SEQ ID NO: 1;
e) A polypeptide comprising amino acids 79 to 244 of SEQ ID NO: 1;
f) A mutein of any of (a) to (e), wherein the amino acid sequence has at least 75%, 80%, 85%, 90%, 95%, 96%, 97%, 98% or 99% identity to at least one of the sequences in (a) to (e);
g) A mutein of any of (a) to (e) which is encoded by a DNA sequence which hybridizes to the complement of the native DNA sequence encoding any of (a) to (e) under moderately stringent conditions or under highly stringent conditions;
h) A mutein of any of (a) to (e) wherein any changes in the amino acid sequence are conservative amino acid substitutions to the amino acid sequences in (a) to (e);
i) A salt or a fused protein, functional derivative, active fraction or circularly permutated derivative of any of (a) to (h).
   wherein said Acrp30g polypeptide does not comprise amino acids 1 to 70 of SEQ ID NO: 1; and wherein said Acrp30 polypeptide has anti-coagulant and/or anti-aggregant activity.

Preferably, the anti-coagulant and/or anti-aggregant activity is assessed by measuring the Howell time, i.e., the Howell time increases in a dose dependent manner upon injection of increasing doses of Acrp30g polypeptides. More preferably, an Acrp30g polypeptide has biological activity if the Howell time is increased of at least 5%, 10%, 15%, 20%, 25%, 30%, 40% or 50% when a dose of 0.3 mg/ml of Acrp30g is injected to a mouse as compared to the control (e.g., a mouse injected with a saline solution). Most preferably, said Howell time is increased of at least 15% when a dose of 0.3 mg/ml of Acrp30g is injected to a mouse as compared to the control.

An Acrp30g polypeptide in accordance with the invention does not comprise amino acids 1 to 70 of SEQ ID NO: 1. Preferably, it does not comprise amino acids 1 to 75, 1 to 80, 1 to 90, 1 to 95, 1 to 100, 1 to 105, 1 to 107, 1 to 109, 1 to 110 or 1 to 113 of SEQ ID NO: 1.

As used herein, a polypeptide consisting of SEQ ID NO: 2 is referred to as Acrp30g-1 and a polypeptide consisting of SEQ ID NO: 3 is referred to as Acrp30g-2.

In one embodiment, the Acrp30g polypeptide in accordance with the present invention is selected from the Acrp30 polypeptides disclosed in WO 01/51645.

In a preferred embodiment of the present invention, the Acrp30g polypeptide in accordance with the present invention corresponds to the 15.4 kDa cleavage product of Acrp30 that is described in Examples 19 and 21. More preferably, the Acrp30g polypeptide in accordance with the present invention comprises a contiguous span of SEQ ID NO: 1 starting at amino acid position 105, 106, 107, 108, 109, 110, 111, 112, 113, or 114 and ending at amino acid position 244 of SEQ ID NO: 1. Most preferably, the Acrp30g polypeptide in accordance with the present invention comprises a contiguous span of SEQ ID NO: 1 starting at amino acid position 107, 108, 109 or 110 and ending at amino acid position 244 of SEQ ID NO: 1.

Alternatively, the Acrp30g polypeptide in accordance with the present invention corresponds to the 20 kDa cleavage product of Acrp30 that is described in Examples 19 and 20. Preferably, the Acrp30g polypeptide in accordance with the present invention comprises a contiguous span of SEQ ID NO: 1 starting at amino acid position 75, 76, 77, 78, 79, 80, 81, 82, 83, 84, 85, 86, 87, 88, 89, 90, 91 or 92 and ending at amino acid position 244 of SEQ ID NO: 1. Most preferably, the Acrp30g polypeptide in accordance with the present invention comprises a contiguous span of SEQ ID NO: 1 starting at amino acid position 78, 79 or 80 and ending at amino acid position 244 of SEQ ID NO: 1.

Such an Acrp30g polypeptide corresponding to the 20 kDa cleavage product of Acrp30 may be obtained, e.g., by carrying out an immunoprecipitation of human plasma. For example, the immunoprecipitation can be carried out using a polyclonal antibody obtained from a mammal immunized by injection of a recombinant polypeptide consisting of amino acids 110 to 244 of SEQ ID NO: 1. Alternatively, the immunoprecipitation can be carried out using Preprotech's biotinylated antibody directed to the globular head of human Acrp30. The 20 kDa cleavage product of Acrp30 can also be mimicked by producing a recombinant polypeptide starting at amino acid position 75, 76, 77, 78, 79, 80, 81, 82, 83, 84, 85, 86, 87, 88, 89, 90, 91 or 92 and ending at amino acid position 244 of SEQ ID NO: 1.

The person skilled in the art will appreciate that splice variants, allelic variants, muteins, fragments, salts, homologues in other species, fused proteins, functional derivatives, active fractions and circularly permutated derivatives of the Acrp30g polypeptides of SEQ ID Nos. 2 or 3 will retain a similar, or even better, biological activity than Acrp30g polypeptides of SEQ ID Nos. 2 or 3

Preferred active fractions have an activity which is equal or better than the activity of Acrp30g polypeptides of SEQ ID Nos. 2 or 3, or which have further advantages, such as a better stability or a lower toxicity or immunogenicity, or they are easier to produce in large quantities, or easier to purify. The person skilled in the art will appreciate that muteins, active fragments and functional derivatives can be generated by cloning the corresponding cDNA in appropriate plasmids and testing them in the co-culturing assay, as mentioned above.

The Acrp30g polypeptides according to the present invention may be glycosylated or non-glycosylated, they may be derived from natural sources, such as body fluids, or they may preferably be produced recombinantly. Recombinant expression may be carried out in prokaryotic expression systems such as E. coli, or in eukaryotic, such as insect cells, and preferably in mammalian expression systems, such as CHO-cells or HEK-cells.

As used herein the term “muteins” refers to analogs of an Acrp30g polypeptide comprising amino acids 114 to 244 of SEQ ID NO: 1 and lacking amino acids 1 to 70 of SEQ ID NO: 1 in which one or more of the amino acid residues of said polypeptide are replaced by different amino acid residues, or are deleted, or one or more amino acid residues are added to the natural sequence of said polypeptide, without changing considerably the activity of the resulting products as compared with the polypeptide of SEQ ID NO: 2 or 3. These muteins are prepared by known synthesis and/or by site-directed mutagenesis techniques, or any other known technique suitable therefore. The term “muteins” encompasses naturally-occurring allelic variants and naturally-occurring splice variants or cleavage products of an Acrp30 polypeptide of SEQ ID NO: 1.

Muteins of an Acrp30g polypeptide comprising amino acids 114 to 244 of SEQ ID NO: 1 and lacking amino acids 1 to 70 of SEQ ID NO: 1, which can be used in accordance with the present invention, or nucleic acid coding thereof, include a finite set of substantially corresponding sequences as substitution peptides or polynucleotides which can be routinely obtained by one of ordinary skill in the art, without undue experimentation, based on the teachings and guidance presented herein.

Muteins in accordance with the present invention include proteins encoded by a nucleic acid, such as DNA or RNA, which hybridizes to DNA or RNA, which encodes an Acrp30g polypeptide comprising amino acids 114 to 244 of SEQ ID NO: 1 and lacking amino acids 1 to 70 of SEQ ID NO: 1, under moderately or highly stringent conditions. The term “stringent conditions” refers to hybridization and subsequent washing conditions, which those of ordinary skill in the art conventionally refer to as “stringent”. See Ausubel et al., Current Protocols in Molecular Biology, supra, Interscience, N.Y., §§6.3 and 6.4 (1987, 1992), and Sambrook et al. (Sambrook, J. C., Fritsch, E. F., and Maniatis, T. (1989) Molecular Cloning: A Laboratory Manual, Cold Spring Harbor Laboratory Press, Cold Spring Harbor, N.Y.).

Without limitation, examples of stringent conditions include washing conditions 12-20° C. below the calculated Tm of the hybrid under study in, e.g., 2×SSC and 0.5% SDS for 5 minutes, 2×SSC and 0.1% SDS for 15 minutes; 0.1×SSC and 0.5% SDS at 37° C. for 30-60 minutes and then, a 0.1×SSC and 0.5% SDS at 68° C. for 30-60 minutes. Those of ordinary skill in this art understand that stringency conditions also depend on the length of the DNA sequences, oligonucleotide probes (such as 10-40 bases) or mixed oligonucleotide probes. If mixed probes are used, it is preferable to use tetramethyl ammonium chloride (TMAC) instead of SSC. See Ausubel, supra.

In a preferred embodiment, any such mutein has at least 40% identity with the sequence of an Acrp30g polypeptide comprising amino acids 114 to 244 of SEQ ID NO: 1 and lacking amino acids 1 to 70 of SEQ ID NO: 1. More preferably, it has at least 50%, 55%, 60%, 65%, 70%, 75%, 80%, 85% or, most preferably, at least 90%, 95%, 96%, 97%, 98% or 99% identity thereto.

In another preferred embodiment, such mutein has at least 40% identity with the sequence of an Acrp30g polypeptide of SEQ ID Nos. 2 or 3. More preferably, it has at least 50%, 55%, 60%, 65%, 70%, 75%, 80%, 85% or, most preferably, at least 90%, 95%, 96%, 97%, 98% or 99% identity thereto.

Identity reflects a relationship between two or more polypeptide sequences or two or more polynucleotide sequences, determined by comparing the sequences. In general, identity refers to an exact nucleotide to nucleotide or amino acid to amino acid correspondence of the two polynucleotide or two polypeptide sequences, respectively, over the length of the sequences being compared.

For sequences where there is not an exact correspondence, a “% identity” may be determined. In general, the two sequences to be compared are aligned to give a maximum correlation between the sequences. This may include inserting “gaps” in either one or both sequences, to enhance the degree of alignment. A % identity may be determined over the whole length of each of the sequences being compared (so-called “global alignment”), that is particularly suitable for sequences of the same or very similar length, or over shorter, defined lengths (so-called “local alignment”), that is more suitable for sequences of unequal length. In the frame of the present invention, the “% of identity” refers to the global percent of identity that has been determined over the whole length of each of the sequences being compared.

Known computer programs may be used to determine whether any particular polypeptide is a percentage identical to a sequence of the present invention. Such algorithms and programs include, e.g. TBLASTN, BLASTP, FASTA, TFASTA, and CLUSTALW (Altschul et al., 1990; Altschul et al., 1997; Higgins et al., 1996; Pearson and Lipman, 1988; Thompson et al., 1994). Protein and nucleic acid sequence homologies are preferably evaluated using the Basic Local Alignment Search Tool (“BLAST”), which is well known in the art (Altschul et al., 1990; Altschul et al., 1997; Karlin and Altschul, 1990).

The BLAST programs identify homologous sequences by identifying similar segments, which are referred to herein as “high-scoring segment pairs,” between a query amino or nucleic acid sequence and a test sequence which is preferably obtained from a protein or nucleic acid sequence database. High-scoring segment pairs are preferably identified (i.e., aligned) by means of a scoring matrix, many of which are known in the art. The scoring matrix used may be the BLOSUM62 matrix (Gonnet et al., 1992; Henikoff and Henikoff, 1993). The PAM or PAM250 matrices may also be used (See, e.g., Schwartz and Dayhoff, eds, (1978) Matrices for Detecting Distance Relationships Atlas of Protein Sequence and Structure, Washington: National Biomedical Research Foundation). The BLAST programs evaluate the statistical significance of all high-scoring segment pairs identified, and preferably selects those segments which satisfy a user-specified threshold of significance, such as a user-specified percent homology. Preferably, the statistical significance of a high-scoring segment pair is evaluated using the statistical significance formula of Karlin (Karlin and Altschul, 1990). The BLAST programs may be used with the default parameters or with modified parameters provided by the user.

A preferred method for determining the best overall match between a query sequence (a sequence of the present invention) and a subject sequence, also referred to as a global sequence alignment, can be determined using the FASTDB computer program based on the algorithm of Brutlag (Brutlag et al., 1990). In a sequence alignment the query and subject sequences are both amino acid sequences. The result of said global sequence alignment is in percent identity. Preferred parameters used in a FASTDB amino acid alignment are: Matrix=PAM 0, k-tuple=2, Mismatch Penalty=1, Joining Penalty=20, Randomization Group=25 Length=0, Cutoff Score=1, Window Size=sequence length, Gap Penalty=5, Gap Size Penalty=0.05, Window Size=247 or the length of the subject amino acid sequence, whichever is shorter.

If the subject sequence is shorter than the query sequence due to N- or C-terminal deletions, not because of internal deletions, the results, in percent identity, must be manually corrected because the FASTDB program does not account for N- and C-terminal truncations of the subject sequence when calculating global percent identity. For subject sequences truncated at the N- and C-termini, relative to the query sequence, the percent identity is corrected by calculating the number of residues of the query sequence that are N- and C-terminal of the subject sequence, that are not matched/aligned with a corresponding subject residue, as a percent of the total bases of the query sequence. Whether a residue is matched/aligned is determined by results of the FASTDB sequence alignment. This percentage is then subtracted from the percent identity, calculated by the above FASTDB program using the specified parameters, to arrive at a final percent identity score. This final percent identity score is what is used for the purposes of the present invention. Only residues to the N- and C-termini of the subject sequence, which are not matched/aligned with the query sequence, are considered for the purposes of manually adjusting the percent identity score. That is, only query amino acid residues outside the farthest N- and C-terminal residues of the subject sequence.

For example, a 90 amino acid residue subject sequence is aligned with a 100-residue query sequence to determine percent identity. The deletion occurs at the N-terminus of the subject sequence and therefore, the FASTDB alignment does not match/align with the first residues at the N-terminus. The 10 unpaired residues represent 10% of the sequence (number of residues at the N- and C-termini not matched/total number of residues in the query sequence) so 10% is subtracted from the percent identity score calculated by the FASTDB program. If the remaining 90 residues were perfectly matched the final percent identity would be 90%.

Preferred changes for muteins in accordance with the present invention are what are known as “conservative” substitutions. Conservative amino acid substitutions of Acrp30g polypeptides in accordance with the present invention may include synonymous amino acids within a group which have sufficiently similar physicochemical properties that substitution between members of the group will preserve the biological function of the molecule (Grantham, 1974). It is clear that insertions and deletions of amino acids may also be made in the above-defined sequences without altering their function, particularly if the insertions or deletions only involve a few amino acids, e.g. under thirty, and preferably under ten, and do not remove or displace amino acids which are critical to a functional conformation, e.g. cysteine residues. Proteins and muteins produced by such deletions and/or insertions come within the purview of the present invention.

Preferably, the synonymous amino acid groups are those defined in Table I. More preferably, the synonymous amino acid groups are those defined in Table II; and most preferably the synonymous amino acid groups are those defined in Table III.

TABLE I
Preferred Groups of Synonymous Amino Acids

| Amino Acid | Synonymous Group |
|---|---|
| Ser | Ser, Thr, Gly, Asn |
| Arg | Arg, Gln, Lys, Glu, His |
| Leu | Ile, Phe, Tyr, Met, Val, Leu |
| Pro | Gly, Ala, Thr, Pro |
| Thr | Pro, Ser, Ala, Gly, His, Gln, Thr |
| Ala | Gly, Thr, Pro, Ala |
| Val | Met, Tyr, Phe, Ile, Leu, Val |
| Gly | Ala, Thr, Pro, Ser, Gly |
| Ile | Met, Tyr, Phe, Val, Leu, Ile |
| Phe | Trp, Met, Tyr, Ile, Val, Leu, Phe |
| Tyr | Trp, Met, Phe, Ile, Val, Leu, Tyr |
| Cys | Ser, Thr, Cys |
| His | Glu, Lys, Gln, Thr, Arg, His |
| Gln | Glu, Lys, Asn, His, Thr, Arg, Gln |
| Asn | Gln, Asp, Ser, Asn |
| Lys | Glu, Gln, His, Arg, Lys |
| Asp | Glu, Asn, Asp |
| Glu | Asp, Lys, Asn, Gln, His, Arg, Glu |
| Met | Phe, Ile, Val, Leu, Met |
| Trp | Trp |

TABLE II
More Preferred Groups of Synonymous Amino Acids

| Amino Acid | Synonymous Group |
|---|---|
| Ser | Ser |
| Arg | His, Lys, Arg |
| Leu | Leu, Ile, Phe, Met |
| Pro | Ala, Pro |
| Thr | Thr |
| Ala | Pro, Ala |
| Val | Val, Met, Ile |
| Gly | Gly |
| Ile | Ile, Met, Phe, Val, Leu |
| Phe | Met, Tyr, Ile, Leu, Phe |
| Tyr | Phe, Tyr |
| Cys | Cys, Ser |
| His | His, Gln, Arg |
| Gln | Glu, Gln, His |
| Asn | Asp, Asn |
| Lys | Lys, Arg |
| Asp | Asp, Asn |
| Glu | Glu, Gln |
| Met | Met, Phe, Ile, Val, Leu |
| Trp | Trp |

TABLE III
Most Preferred Groups of Synonymous Amino Acids

| Amino Acid | Synonymous Group |
|---|---|
| Ser | Ser |
| Arg | Arg |
| Leu | Leu, Ile, Met |
| Pro | Pro |
| Thr | Thr |
| Ala | Ala |
| Val | Val |
| Gly | Gly |
| Ile | Ile, Met, Leu |
| Phe | Phe |
| Tyr | Tyr |
| Cys | Cys, Ser |
| His | His |
| Gln | Gln |
| Asn | Asn |
| Lys | Lys |
| Asp | Asp |
| Glu | Glu |
| Met | Met, Ile, Leu |
| Trp | Trp |

Examples of production of amino acid substitutions in polypeptides which can be used for obtaining muteins of an Acrp30g polypeptide of SEQ ID Nos. 2 or 3 include any known method steps, such as presented in U.S. Pat. Nos. 4,959,314, 4,588,585 and 4,737,462, to Mark et al; 5,116,943 to Koths et al., 4,965,195 to Namen et al; 4,879,111 to Chong et al; and 5,017,691 to Lee et al; and lysine substituted proteins presented in U.S. Pat. No. 4,904,584 (Shaw et al).

The term “fused protein” refers to a polypeptide comprising amino acids 114 to 244 of SEQ ID NO: 1 and lacking amino acids 1 to 70 of SEQ ID NO: 1 or a mutein thereof fused with another protein, which e.g. has an extended residence time in body fluids. The Acrp30g moiety may be fused to another protein, polypeptide or the like, e.g. an immunoglobulin or a fragment thereof. Immunoglobulin Fc portions are particularly suitable for production of di- or multi-meric Ig fusion proteins. The Acrp30g moiety in accordance with the present invention may e.g. be linked to portions of an immunoglobulin in such a way as to produce an Acrp30g polypeptide dimerized by the Ig Fc portion. Alternatively, the sequence of the Acrp30g moiety is fused to a signal peptide and/or to a leader sequence allowing enhanced secretion. The leader sequence may for example corresponds to the IgSP-tPA pre-propeptide disclosed in PCT application PCT/EP2004/052302.

In a preferred embodiment, the Acrp30g polypeptide in accordance with the present invention is a fused protein comprising a carrier molecule, a peptide or a protein that promotes the crossing of the blood brain barrier, and/or comprising a carrier molecule, a peptide or a protein that increases half-life.

The fusion may be direct, or via a short linker peptide which can be as short as 1 to 3 amino acid residues in length or longer, for example, 13 amino acid residues in length. Said linker may be a tripeptide of the sequence E-F-M (Glu-Phe-Met), for example, or a 13-amino acid linker sequence comprising Glu-Phe-Gly-Ala-Gly-Leu-Val-Leu-Gly-Gly-Gln-Phe-Met introduced between the Acrp30g sequence and the immunoglobulin sequence, for instance. The resulting fusion protein has improved properties, such as an extended residence time in body fluids (half-life), or an increased specific activity, increased expression level. The Ig fusion may also facilitate purification of the fused protein.

In a further preferred embodiment of the invention, the fused protein comprises an immunoglobulin (Ig) domain.

In a yet another preferred embodiment, the Acrp30g polypeptide in accordance with the present invention is a fused protein comprising the constant region of an Ig molecule. Preferably, it is fused to heavy chain regions, like the CH2 and CH3 domains of human IgG1, for example. Other isoforms of Ig molecules are also suitable for the generation of fusion proteins according to the present invention, such as isoforms IgG₂ or IgG₄, or other Ig classes, like IgM, for example. Fused proteins may be monomeric or multimeric, hetero- or homomultimeric. The immunoglobulin portion of the fused protein may be further modified in a way as to not activate complement binding or the complement cascade or bind to Fc-receptors.

Fused proteins may also be prepared by fusing the Acrp30g moiety with domains isolated from other proteins allowing the formation or dimers, trimers, etc. Examples for protein sequences allowing the multimerization of the polypeptides of the Invention are domains isolated from proteins such as hCG (WO 97/30161), collagen X (WO 04/33486), C4BP (WO 04/20639), Erb proteins (WO 98/02540), or coiled coil peptides (WO 01/00814).

Accordingly, a further preferred embodiment of the invention is directed to a fused protein comprises an hCG domain.

“Functional derivatives” as used herein, cover derivatives of a polypeptide comprising amino acids 114 to 244 of SEQ ID NO: 1 and lacking amino acids 1 to 70 of SEQ ID NO: 1 or a mutein thereof, which may be prepared from the functional groups which occur as side chains on the residues or the N- or C-terminal groups, by means known in the art, and are included in the invention as long as they remain pharmaceutically acceptable, i.e. they do not destroy the activity of the protein which is substantially similar to the activity of a polypeptide of SEQ ID Nos. 2 or 3, and do not confer toxic properties on compositions containing it.

These derivatives may, for example, include polyethylene glycol side-chains, which may mask antigenic sites and extend the residence of a naturally occurring Acrp30g polypeptide in body fluids. Other derivatives include aliphatic esters of the carboxyl groups, amides of the carboxyl groups by reaction with ammonia or with primary or secondary amines, N-acyl derivatives of free amino groups of the amino acid residues formed with acyl moieties (e.g. alkanoyl or carbocyclic aroyl groups) or O-acyl derivatives of free hydroxyl groups (for example that of seryl or threonyl residues) formed with acyl moieties.

As “active fractions” of a polypeptide comprising amino acids 114 to 244 of SEQ ID NO: 1 and lacking amino acids 1 to 70 of SEQ ID NO: 1 or a mutein thereof, the present invention covers any fragment or precursors of the polypeptide chain of the protein molecule alone or together with associated molecules or residues linked thereto, e.g. sugar or phosphate residues, or aggregates of the protein molecule or the sugar residues by themselves, provided said fraction has substantially similar activity to a an Acrp30g polypeptide of SEQ ID Nos. 2 or 3.

The term “salts” herein refers to both salts of carboxyl groups and to acid addition salts of amino groups of a polypeptide comprising amino acids 114 to 244 of SEQ ID NO: 1 and lacking amino acids 1 to 70 of SEQ ID NO: 1 or a mutein thereof. Salts of a carboxyl group may be formed by means known in the art and include inorganic salts, for example, sodium, calcium, ammonium, ferric or zinc salts, and the like, and salts with organic bases as those formed, for example, with amines, such as triethanolamine, arginine or lysine, piperidine, procaine and the like. Acid addition salts include, for example, salts with mineral acids, such as, for example, hydrochloric acid or sulfuric acid, and salts with organic acids, such as, for example, acetic acid or oxalic acid. Of course, any such salts must retain the biological activity of an Acrp30g polypeptide of SEQ ID Nos. 2 or 3.

Functional derivatives may be conjugated to polymers in order to improve the properties of the protein, such as the stability, half-life, bioavailability, tolerance by the human body, or immunogenicity. To achieve this goal, the Acrp30g polypeptide may be linked e.g. to Polyethlyenglycol (PEG). PEGylation may be carried out by known methods, described in WO 92/13095, for example.

Therefore, in a preferred embodiment of the present invention, the Acrp30g polypeptide in accordance with the present invention is PEGylated.

In a preferred embodiment, the Acrp30g polypeptide in accordance with the present invention is composed of at least 85%, 90%, 95%, 96%, 97%, 98% or 99% trimeric species.

The invention further relates to the simultaneous, sequential, or separate use of:
(i) an Acrp30g polypeptide or of an agonist thereof; and
(ii) an anti-coagulant agent and/or anti-aggregant agent different from said Acrp30g polypeptide or agonist thereof;
   for the manufacture of a medicament for the treatment and/or the prevention of a disease selected from the group consisting of a thrombosis-related disease, an hypertensive disorder of the pregnancy, tumor implantation, tumor seeding and metastasis. The anti-coagulant agent different from said Acrp30g polypeptide or agonist thereof may correspond to any drug that inactivates thrombin or other clotting factors. Such anti-coagulant agents include, e.g., heparin, hirudin, warfarin, dicumarol and derivatives thereof. The anti-aggregant agent may correspond to any antiplatelet drug. For example, anti-aggregant agent may correspond to aspirin, glucoprotein IIb/IIIa inhibitors or thienopyridines such as, e.g., clopidogrel and ticlopidine.

The invention further relates to the simultaneous, sequential, or separate use of:
(iii) an Acrp30g polypeptide or of an agonist thereof; and
(iv) a fibrinolytic agent;
   for the manufacture of a medicament for the treatment and/or the prevention of a disease selected from the group consisting of a thrombosis-related disease, an hypertensive disorder of the pregnancy, tumor implantation, tumor seeding and metastasis. The fibrinolytic agent may correspond to any drug that promotes degradation of fibrin into soluble peptides. Such fibrinolytic agents include, e.g., streptokinase, urokinase and derivatives thereof.

The invention further relates to the simultaneous, sequential, or separate use of an Acrp30g polypeptide or of an agonist thereof and percutaneous transluminal angioplasty for the treatment of a thrombosis-related disease.

The invention further relates to the simultaneous, sequential, or separate use of an Acrp30g polypeptide or of an agonist thereof and a surgical intervention for the treatment of a disease selected from the group consisting of a thrombosis-related disease, an hypertensive disorder of the pregnancy, tumor implantation, tumor seeding and metastasis.

The invention further relates to the simultaneous, sequential, or separate use of:
(v) an Acrp30g polypeptide or of an agonist thereof; and
(vi) a drug for the treatment of cancer;
   for the manufacture of a medicament for the treatment and/or the prevention of a disease selected from the group consisting of tumor implantation, tumor seeding and metastasis. Numerous drugs for the treatment of cancer are known in the art and may be used in the frame of the present embodiment. These drugs include those used in chemotherapies, targeted therapies (e.g., radioactive monoclonal antibodies and tyrosine kinase inhibitors), biological therapies (e.g., interferons, interleukins, monoclonal antibodies, colony stimulating factors, cytokines and vaccines) and hormonal therapies. The use of an Acrp30g polypeptide or of an agonist thereof may also be associated with surgery and/or a radiation therapy using high-energy rays to damage or kill cancer cells, or with stem-cell transplantation.

In a preferred embodiment of the present invention, the Acrp30g polypeptide in accordance with the present invention is used in an amount of:
a) about 0.01 to 10 mg/kg of body weight; or
b) about 0.1 to 1 mg/kg of body weight; or
c) about 9, 8, 7, 6, 5, 4, 3, 2 or 1 mg/kg of body weight.

A fourth aspect of the present invention relates to the use of a nucleic acid molecule for manufacture of a medicament for the treatment and/or prevention of a disease selected from the group consisting of a thrombosis-related disease, an hypertensive disorder of the pregnancy, tumor implantation, tumor seeding and metastasis, wherein the nucleic acid molecule comprises a nucleic acid sequence encoding an Acrp30g polypeptide in accordance with the present invention.

The nucleic acid may e.g. be administered as a naked nucleic acid molecule, e.g. by intramuscular injection.

It may further comprise vector sequences, such as viral sequence, useful for expression of the gene encoded by the nucleic acid molecule in the human body, preferably in the appropriate cells or tissues.

Therefore, in a preferred embodiment, the nucleic acid molecule further comprises an expression vector sequence. Expression vector sequences are well known in the art, they comprise further elements serving for expression of the gene of interest. They may comprise regulatory sequence, such as promoter and enhancer sequences, selection marker sequences, origins of multiplication, and the like. A gene therapeutic approach is thus used for treating and/or preventing the disease. Advantageously, the expression of the Acrp30g polypeptide in accordance with the present invention will then be in situ.

In a preferred embodiment of the invention, the expression vector may be administered by intramuscular injection.

The use of a vector for inducing and/or enhancing the endogenous production of Acrp30g, or of an agonist thereof, in a cell in the manufacture of a medicament for the treatment and/or prevention of a disease selected from the group consisting of a thrombosis-related disease, an hypertensive disorder of the pregnancy, tumor implantation, tumor seeding and metastasis is further encompassed by the present invention. Preferably, the cell is normally silent for expression of said Acrp30g polypeptide, or expresses amounts of said Acrp30g polypeptide which are not sufficient for allowing industrial production of a recombinant protein. The vector may comprise regulatory sequences functional in the cells desired to express the Acrp30g polypeptide in accordance with the present invention. Such regulatory sequences may be promoters or enhancers, for example. The regulatory sequence may then be introduced into the appropriate locus of the genome by homologous recombination, thus operably linking the regulatory sequence with the gene, the expression of which is required to be induced or enhanced. The technology is usually referred to as “endogenous gene activation” (EGA), and it is described e.g. in WO 91/09955.

A sixth aspect of the invention relates to the use of a cell that has been genetically modified to produce an Acrp30g polypeptide in accordance with the invention in the manufacture of a medicament for the treatment and/or prevention of a disease selected from the group consisting of a thrombosis-related disease, an hypertensive disorder of the pregnancy, tumor implantation, tumor seeding and metastasis. Thus, a cell therapeutic approach may be used in order to deliver the drug to the appropriate parts of the human body.

The invention further relates to pharmaceutical compositions, particularly useful for prevention and/or treatment of a disease selected from the group consisting of a thrombosis-related disease, an hypertensive disorder of the pregnancy, tumor implantation, tumor seeding and metastasis, which comprise:
a) a therapeutically effective amount of an Acrp30g polypeptide in accordance with the invention, or of an agonist thereof; and
b) a pharmaceutically acceptable carrier.

The definition of “pharmaceutically acceptable carrier” is meant to encompass any carrier, which does not interfere with effectiveness of the biological activity of the active ingredient and that is not toxic to the host to which it is administered. For example, for parenteral administration, the active protein(s) may be formulated in a unit dosage form for injection in vehicles such as saline, dextrose solution, serum albumin and Ringer's solution.

The active ingredients of the pharmaceutical composition according to the invention can be administered to an individual in a variety of ways. The routes of administration include intradermal, transdermal (e.g. in slow release formulations), intramuscular, intraperitoneal, intravenous, subcutaneous, oral, epidural, topical, intrathecal, rectal, and intranasal routes. Any other therapeutically efficacious route of administration can be used, for example absorption through epithelial or endothelial tissues or by gene therapy wherein a DNA molecule encoding the active agent is administered to the patient (e.g. via a vector), which causes the active agent to be expressed and secreted in vivo. In addition, the protein(s) according to the invention can be administered together with other components of biologically active agents such as pharmaceutically acceptable surfactants, excipients, carriers, diluents and vehicles.

For parenteral (e.g. intravenous, subcutaneous, intramuscular) administration, the active protein(s) can be formulated as a solution, suspension, emulsion or lyophilized powder in association with a pharmaceutically acceptable parenteral vehicle (e.g. water, saline, dextrose solution) and additives that maintain isotonicity (e.g. mannitol) or chemical stability (e.g. preservatives and buffers). The formulation is sterilized by commonly used techniques.

The bioavailability of the active protein(s) according to the invention can also be ameliorated by using conjugation procedures which increase the half-life of the molecule in the human body, for example linking the molecule to polyethylenglycol, as described in the PCT Patent Application WO 92/13095.

The therapeutically effective amounts of the active protein(s) will be a function of many variables, including the type of protein, the affinity of the protein, any residual cytotoxic activity exhibited by the antagonists, the route of administration, the clinical condition of the patient (including the desirability of maintaining a non-toxic level of endogenous Acrp30g activity).

A “therapeutically effective amount” is such that when administered, the Acrp30g polypeptide in accordance with the present invention exerts a beneficial effect on the disease selected from the group consisting of a thrombosis-related disease, an hypertensive disorder of the pregnancy, tumor implantation, tumor seeding and metastasis. The dosage administered, as single or multiple doses, to an individual will vary depending upon a variety of factors, including Acrp30g pharmacokinetic properties, the route of administration, patient conditions and characteristics (sex, age, body weight, health, size), extent of symptoms, concurrent treatments, frequency of treatment and the effect desired.

The Acrp30g polypeptide in accordance with the invention can preferably be used in an amount of about 0.01 to 10 mg/kg or about 0.05 to 5 mg/kg or body weight or about 0.1 to 3 mg/kg of body weight or about 1 to 2 mg/kg of body weight. Further preferred amounts of Acrp30g polypeptides are amounts of about 0.1 to 1000 μg/kg of body weight or about 1 to 100 μg/kg of body weight or about 10 to 50 μg/kg of body weight.

The route of administration is preferably parenteral. The Acrp30g polypeptide in accordance with the present invention may be administered, e.g., by subcutaneous, intravenous or intramuscular route.

In further preferred embodiments, the Acrp30g polypeptide in accordance with the invention is administered daily or every other day.

The daily doses are usually given in divided doses or in sustained release form effective to obtain the desired results. Second or subsequent administrations can be performed at a dosage which is the same, less than or greater than the initial or previous dose administered to the individual. A second or subsequent administration can be administered during or prior to onset of the disease.

According to the invention, the Acrp30g polypeptide in accordance with the invention can be administered prophylactically or therapeutically to an individual prior to, simultaneously or sequentially with other therapeutic regimens or agents (e.g. multiple drug regimens), in a therapeutically effective amount. Active agents that are administered simultaneously with other therapeutic agents can be administered in the same or different compositions.

In a seventh aspect, the invention further relates to a method for treating a disease selected from the group consisting of a thrombosis-related disease, an hypertensive disorder of the pregnancy, tumor implantation, tumor seeding and metastasis comprising administering to a patient in need thereof an effective amount of an Acrp30g polypeptide in accordance with the invention, or of an agonist thereof, optionally together with a pharmaceutically acceptable carrier.

In such a method, the Acrp30g polypeptide or agonist thereof may be administered together with a polypeptide selected from the group consisting of an anti-coagulant agent and/or anti-aggregant agent different from said Acrp30g polypeptide, a fibrinolytic agent and a drug for the treatment of cancer.

In a eighth aspect, the invention further relates to an antibody specifically binding to an Acrp30 fragment characterized by a mass of about 15.4 kDa and/or about 20 kDa. Such an antibody in accordance with the invention does not bind to full-length Acrp30.

A first embodiment is directed to an anti-Acrp30g-bth antibody characterized in that:
(i) said antibody specifically binds to an Acrp30g polypeptides of about 15.4 kDa;
(ii) said antibody specifically binds to an Acrp30g polypeptides of about 20 kDa; and
(iii) said antibody does not bind to full-length Acrp30.

A second embodiment is directed to an anti-Acrp30g-15.4 antibody characterized in that:
(i) said antibody specifically binds to an Acrp30g polypeptides of about 15.4 kDa;
(ii) said antibody specifically does not bind to an Acrp30g polypeptides of about kDa; and
(iii) said antibody does not bind to full-length Acrp30.

A third embodiment is directed to an anti-Acrp30g-20 antibody characterized in that:
(i) said antibody specifically binds to an Acrp30g polypeptides of about 20 kDa;
(ii) said antibody specifically does not bind to an Acrp30g polypeptides of about 15.4 kDa; and
(iii) said antibody does not bind to full-length Acrp30.

The antibodies of the present invention may be monoclonal or polyclonal. The antibodies of the present invention include monoclonal and polyclonal antibodies. The antibodies of the present invention include IgG (including IgG1, IgG2, IgG3, and IgG4), IgA (including IgA1 and IgA2), IgD, IgE, or IgM, and IgY. The term “antibody” (Ab) refers to a polypeptide or group of polypeptides which are comprised of at least one binding domain, where a binding domain is formed from the folding of variable domains of an antibody compound to form three-dimensional binding spaces with an internal surface shape and charge distribution complementary to the features of an antigenic determinant of an antigen, which allows an immunological reaction with the antigen. As used herein, the term “antibody” is meant to include whole antibodies, including single-chain whole antibodies, and antigen binding fragments thereof. In a preferred embodiment the antibodies are human antigen binding antibody fragments of the present invention include, but are not limited to, Fab, Fab′ F(ab)2 and F(ab′)2, Fd, single-chain Fvs (scFv), single-chain antibodies, disulfide-linked Fvs (sdFv) and fragments comprising either a V_{L} or V_{H} domain. The antibodies may be from any animal origin including birds and mammals. Preferably, the antibodies are from human, mouse, rabbit, goat, guinea pig, camel, horse or chicken. The present invention further includes humanized and human antibodies

The invention further relates to uses of such antibodies in accordance with the invention for diagnostic purposes.

In one embodiment, the present invention pertains to the use of an anti-Acrp30g-bth and/or of an anti-Acrp30g-15.4 antibody for determining whether an individual suffers from or is at risk of suffering from a disease selected from the group consisting of a thrombosis-related disease, an hypertensive disorder of the pregnancy, tumor implantation, tumor seeding and metastasis.

In another embodiment, the present invention pertains to the use of an anti-Acrp30g-bth and/or of an anti-Acrp30g-20 antibody for determining whether an individual suffers from or is at risk of suffering from a metabolic disorder.

As used herein, the term “metabolic disorder” encompasses obesity, type II diabetes, insulin resistance, hypercholesterolemia, hyperlipidemia, dyslipidemia, syndrome X, and atherosclerosis. The terms “obesity”, “type II diabetes”, “insulin resistance”, “hypercholesterolemia”, “hyperlipidemia”, “dyslipidemia” and “atherosclerosis” refer to conditions defined in “The Merck Manual—Second Home Edition” (Publisher: Merck & Co). The term “syndrome X” refers to a constellation of atherosclerotic risk factors, including insulin resistance, hyperinsulinemia, dyslipidemia, hypertension and obesity (Roth et al., 2002).

In a ninth aspect, the invention relates to diagnostic kits comprising antibodies in accordance with the invention.

One embodiment provides a diagnostic kit for determining whether an individual suffers from or is at risk of suffering from a disease selected from the group consisting of a thrombosis-related disease, an hypertensive disorder of the pregnancy, tumor implantation, tumor seeding and metastasis, characterised by the fact that it comprises an anti-Acrp30g-bth and/or an anti-Acrp30g-15.4 antibody.

Another embodiment provides a diagnostic kit for determining whether an individual suffers from or is at risk of suffering from a metabolic disorder, characterised by the fact that it comprises an anti-Acrp30g-bth and/or an anti-Acrp30g-20 antibody.

The kit in accordance with the present invention comprises an antibody in accordance with the present invention and reagents. Preferably, the antibody in accordance with the present invention is labeled. Alternatively, the antibody in accordance with the present invention is not labeled and the kit comprises a labeled secondary antibody binding to the antibody in accordance with the present invention.

In a tenth aspect, the invention relates to methods of diagnosing a disease selected from the group consisting of a thrombosis-related disease, an hypertensive disorder of the pregnancy, a metabolic disease, tumor implantation, tumor seeding and metastasis, in which either the presence or the absence, or the levels, of an Acrp30g polypeptide of about 15.4 kDa or of about 20 kDa is assessed in a plasma sample.

In one embodiment, the invention provides a method of diagnosing a disease selected from the group consisting of a thrombosis-related disease, an hypertensive disorder of the pregnancy, tumor implantation, tumor seeding and metastasis comprising determining the presence or the absence of an Acrp30g polypeptide of about 15.4 kDa in a plasma test sample from an individual.

In another embodiment, the invention provides a method of diagnosing a metabolic disorder comprising the steps of determining the presence or the absence of an Acrp30g polypeptide of about 20 kDa in a plasma test sample from an individual wherein the absence of Acrp30g polypeptide of about 20 kDa in said plasma test sample provides an indication that said individual suffers from or is at risk of suffering from said metabolic disorder. Such a method may be performed, e.g., as described in Example 20.

In another embodiment, the invention provides a method of diagnosing disease selected from the group consisting of a thrombosis-related disease, an hypertensive disorder of the pregnancy, tumor implantation, tumor seeding and metastasis in an individual, comprising the steps of:
(i) detecting the levels of an Acrp30g polypeptide of about 15.4 kDa in a plasma test sample of tissue cells obtained from said individual; and
(ii) detecting the levels of said Acrp30g polypeptide of about 15.4 kDa in a plasma control sample.
   wherein a statistically significant change in levels of said Acrp30g polypeptide of about 15.4 kDa in said test sample compared to the levels in said control sample indicates that said individual suffers from or is at risk of suffering from said disease. Preferably, the levels of said Acrp30g polypeptide of about 15.4 kDa are detected using an antibody. Most preferably, the levels of said Acrp30g polypeptide of about 15.4 kDa are detected using an anti-Acrp30g and/or an anti-Acrp30g-15.4 antibody in accordance with the present invention.

In another embodiment, the invention provides a method of diagnosing a metabolic disease in an individual, comprising the steps of:
(i) detecting the levels of an Acrp30g polypeptide of about 20 kDa in a plasma test sample of tissue cells obtained from said individual; and
(ii) detecting the levels of said Acrp30g polypeptide of about 20 kDa in a plasma control sample.
   wherein a statistically significant change in levels of said Acrp30g polypeptide of about 20 kDa in said test sample compared to the levels in said control sample indicates that said individual suffers from or is at risk of suffering from said disease. Preferably, the levels of said Acrp30g polypeptide of about 20 kDa are detected using an antibody. Most preferably, the levels of said Acrp30g polypeptide of about 20 kDa are detected using an anti-Acrp30g and/or an anti-Acrp30g-20 antibody in accordance with the present invention.

It has been shown in the frame of the present invention that the presence of the Acrp30 cleavage product of 20 kDa in plasma is correlated with free fatty acid levels and resting energy expenditure in obese individuals (Example 20).

Thus, in an eleventh aspect, the invention contemplates the use of a polypeptide for the manufacture of a medicament for the treatment and/or the prevention of a metabolic disorder characterized in that said polypeptide comprises a fragment of Acrp30 of about 20 kDa. Such a fragment of Acrp30 of about 20 kDa preferably consists of a contiguous span of SEQ ID NO: 1 starting at amino acid position 75, 76, 77, 78, 79, 80, 81, 82, 83, 84, 85, 86, 87, 88, 89, 90, 91 or 92 and ending at amino acid position 244 of SEQ ID NO: 1. Most preferably, such a fragment of Acrp30 of about 20 kDa consists of a contiguous span of SEQ ID NO: 1 starting at amino acid position 78, 79 or 80 and ending at amino acid position 244 of SEQ ID NO: 1.

In the context of this aspect, the polypeptide comprising a fragment of Acrp30 of about 20 kDa is not required to exhibit an anti-aggregant and/or anti-coagulant activity, but must exhibit an activity selected from the group consisting of stimulation of free fatty acid oxidation, stimulation of muscle lipid oxidation, stimulation of lipid partitioning and stimulation of lipid metabolism. Methods for measuring such activities are well-known in the art and are disclosed, e.g., in WO0151645.

All references cited herein, including journal articles or abstracts, published or unpublished U.S. or foreign patent application, issued U.S. or foreign patents or any other references, are entirely incorporated by reference herein, including all data, tables, figures and text presented in the cited references. Additionally, the entire contents of the references cited within the references cited herein are also entirely incorporated by reference.

Reference to known method steps, conventional methods steps, known methods or conventional methods is not any way an admission that any aspect, description or embodiment of the present invention is disclosed, taught or suggested in the relevant art.

The foregoing description of the specific embodiments will so fully reveal the general nature of the invention that others can, by applying knowledge within the skill of the art (including the contents of the references cited herein), readily modify and/or adapt for various application such specific embodiments, without undue experimentation, without departing from the general concept of the present invention. Therefore, such adaptations and modifications are intended to be within the meaning range of equivalents of the disclosed embodiments, based on the teaching and guidance presented herein. It is to be understood that the phraseology or terminology herein is for the purpose of description and not of limitation, such that the terminology or phraseology of the present specification is to be interpreted by the skilled artisan in light of the teachings and guidance presented herein, in combination with the knowledge of one of ordinary skill in the art.

Having now described the invention, it will be more readily understood by reference to the following examples that are provided by way of illustration and are not intended to be limiting of the present invention.

### EXAMPLES

### Example 1

### Effect of Acrp30g on the Volume of Blood Collected by Retroorbital Puncture or by Decapitation in db/db Mice

Material and Methods

Polypeptides of SEQ ID NO: 2 (Acrp30g-1) and of SEQ ID NO: 3 (Acrp30g-2) were produced in E. coli.

Acrp30g-1 and Acrp30g-2 were administered to db/db mice (diabetic mice) at the doses of 30 and 100 μg/kg, twice daily during 4 or 5 days, by subcutaneous route. The last day, the mice were sacrificed either by exanguination using retroorbital puncture or by decapitation. The blood was collected and weighted. Each experimental group had 6 to 8 mice.

Results

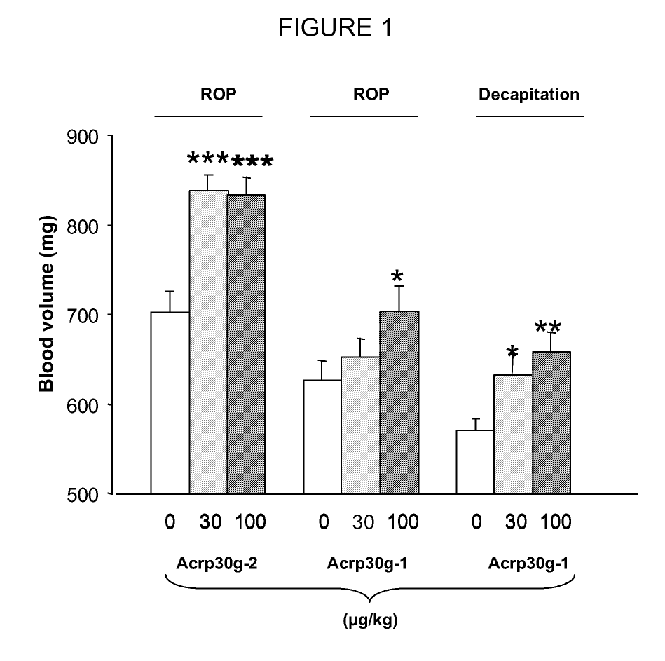

Table 1, the data of which are shown as FIG. 1, demonstrates that Acrp30g-1 and Acrp30g-2 significantly increased the blood volume collected by retroorbital puncture or by decapitation in a dose dependent manner.

TABLE 1

| | | Dose | Nos. of | Nos. of | Blood volume | | |
|---|---|---|---|---|---|---|---|
| Compound | Route | (μg/kg) | injections | mice | (mg) | p | % increase |
| Acrp30g-2 | puncture | 0 | 9 | 8 | 703 ± 23 | — | — |
| | | 30 | 9 | 8 | 838 ± 17 | 0.001 | 19% |
| | | 100 | 9 | 8 | 834 ± 19 | 0.001 | 19% |
| Acrp30g-1 | | 0 | 9 | 8 | 626 ± 22 | — | — |
| | | 30 | 9 | 7 | 653 ± 19 | ns | 4% |
| | | 100 | 9 | 6 | 704 ± 28 | 0.05 | 13% |
| Acrp30g-1 | decapitation | 0 | 8 | 8 | 571 ± 13 | — | — |
| | | 30 | 8 | 8 | 632 ± 23 | 0.05 | 11% |
| | | 100 | 8 | 8 | 658 ± 22 | 0.01 | 15% |

Conclusion

Daily treatments of normal or db/db mice with Acrp30g-1 or Acrp30g-2 increased the blood volume recovered after bleeding.

### Example 2

### Effect of Acrp30g-2 on the Howell Time in C57BL/6 Mice

Introduction

The Howell time allows assessing the anti-aggregant and/or anti-coagulant effect of a compound. It corresponds to the time of coagulation after recalcification of a platelet rich plasma (PRP). This test explores the whole coagulation cascade including the primary hemostasis (ex vivo platelet aggregation) and secondary hemostasis (fibrin formation).

Material and Methods

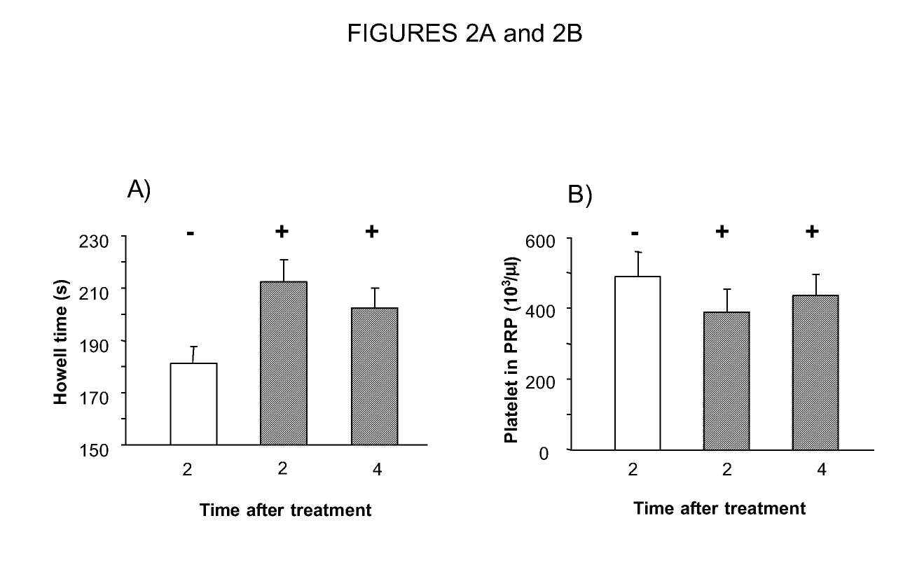

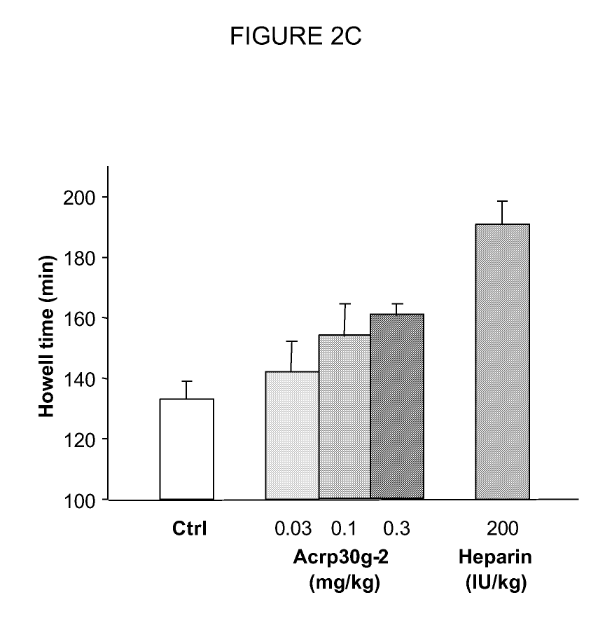

Acrp30g-2 was administered to C57BL/6 mice (normal mice) at the dose of 100 μg/kg, by subcutaneous route. Two or four hours later, the mice were sacrificed by exanguination using an intracardiac puncture under isoflurane anaesthesia. Each experimental group had 11 to 12 mice. The blood was collected in vials containing citrate as an anti-coagulant. PRP was obtained from citrated blood by centrifugation (250 g×10 min). The platelets in PRP were counted using a Beckman-Coulter counter. The Howell time was determined as the time to get coagulation when 100 ml of 25 mM CaCl₂ was incubated with 100 MI of PRP at 37° C. The results are shown on Table 2A, FIG. 2A and FIG. 2C.

A second experiment was performed either with Acrp30g-2 doses of 30, 100 or 300 μg/kg, or with a heparin dose of 200 IU/kg. The mice were sacrificed 2 hours later. Each experimental group had 8 mice. The results are shown on Table 2B and FIG. 2C.

Results

The Howell time was significantly increased when Acrp30g-2 was injected 2 or 4 hours before measurement. The number of platelets in PRP was not significantly affected by the treatment with Acrp30g-2. Acrp30g-2 (30, 100 and 300 μg/kg, sc) increased the Howell time as a dose dependent manner (+7%, +15% & +21%, respectively). Heparin (200 IU/kg) increased it by 43%.

TABLE 2A

| | | | Hours | | | | |
|---|---|---|---|---|---|---|---|
| | Dose | Nos. | After | | | Platelet | |
| | μg/kg, | of | treat- | Howell | | in PRP | |
| Compound | sc | mice | ment | times | p | 103/μl | p |
| Acrp30g-2 | 0 | 11 | 2 | 181 ± 6 | — | 491 ± 68 | — |
| | 100 | 12 | 2 | 213 ± 8 | 0.01 | 389 ± 64 | ns |
| | 100 | 12 | 4 | 203 ± 8 | 0.05 | 437 ± 58 | ns |

TABLE 2B

| | | Nos. of | Howell time | |
|---|---|---|---|---|
| Compound | Dose | mice | s | p |
| Acrp30g-2 | 0 | 8 | 133 ± 6 | — |
| | 30 | 8 | 143 ± 10 | ns |
| | 100 | 8 | 154 ± 11 | 0.1 |
| | 300 | 8 | 161 ± 4 | 0.01 |
| Heparin | 200 | 8 | 191 ± 8 | 0.001 |

### Example 3

### Effect of Acrp30g-2 on the Howell Time in db/db Mice

Material and Methods

Acrp30g-2 was administered to db/db mice at the dose of 10, 30 or 100 μg/kg, by subcutaneous route. Two hours later, the mice were sacrificed by exanguination using an intracardiac puncture under isoflurane anaesthesia. The blood was collected in vials containing citrate as anti-coagulant. Each experimental group contained 12 mice. PRP was obtained from citrated blood by centrifugation (250 g×10 min). The Howell time was determined as the time to get coagulation when 25 mM CaCl₂ (100 μl) was incubated with PRP (100 μl) at 37° C.

Results

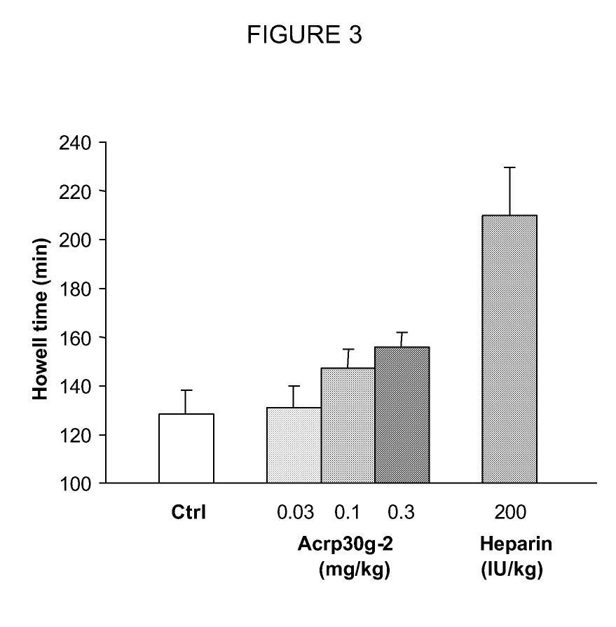

The results are shown in Table 3 and FIG. 3. Acrp30g-2 (30, 100 and 300 μg/kg, sc) increased the Howell time as a dose dependent manner (+2%, +15% & +22%, respectively). Heparin (200 IU/kg) increased it by 64%.

TABLE 3

| | | Nos. of | Howell time | |
|---|---|---|---|---|
| Compound | Dose | mice | s | p |
| Acrp30g-2 | 0 | 11 | 128 ± 10 | — |
| | 30 | 12 | 131 ± 9 | ns |
| | 100 | 12 | 147 ± 8 | ns |
| | 300 | 12 | 156 ± 6 | 0.05 |
| Heparin | 200* | 11 | 210 ± 20 | 0.01 |

### Example 3

### Antiagregggant/anti-Coagulant and Prohemorrhagic Properties of a Repeated Treatment with Acrp30g-2 in db/db Mice

Material and Methods

Acrp30g-2 was administered to db/db mice at the dose of 100 μg/kg, twice daily during 5 days, by subcutaneous route. Two hours after the last injection the mice were exanguinated by intracardiac puncture under isoflurane anaesthesia. The blood was collected in vials containing citrate as anti-coagulant. Each experimental group had 6 to 8 mice.

Anti-aggregant/anti-coagulant effects were assessed by the Howell time. PRP was obtained from citrated blood by centrifugation (250 g×10 min). The Howell time was determined as the time to get coagulation when 100 ml of 25 mM CaCl₂ was incubated with 100 ml of PRP at 37° C.

Prohemorrhagic effects were assessed by the search of blood in feces using Hemocult®, and the determination of the red blood cell and platelet related parameters using a Beckman-Coulter counter.

Results

The results are shown in Table 4. The Howell time was significantly increased by a daily treatment with Acrp30g-2 (100 μg/kg, subcutaneous injection). The number of platelets in PRP was not significantly affected by this treatment. These results showed anti-aggregant and/or anti-coagulant properties of Acrp30g-2.

Should Acrp30g-2 induce hemorrhagic effects in the gastrointestinal tractus, physiological parameters would change, e.g., one would observe a decreased blood hemoglobin, erythrocytes and hematocrit, presence of reticulocytes in blood or presence of blood in feces). None of these parameters were affected by Acrp30g-2. Only the mean corpuscular volume and the red cell distribution width were weakly changed. These low changes do not have any pathophysiological significance. These data thus showed that Acrp30g-2 was devoid of prohemorrhagic properties.

TABLE 4A

| | | | | |
|---|---|---|---|---|
| Parameter | Unit | Control (n = 6) | Acrp30g-2 (n = 8) | p |

Platelet Rich Plasma

| | | | | |
|---|---|---|---|---|
| Howell time | s | 208 ± 6 | 242 ± 14 | 0.056 |
| Platelet in PRP | 10³/μl | 308 ± 65 | 205 ± 45 | ns |

Blood parameter
Platelets

| | | | | |
|---|---|---|---|---|
| PLT | 10³/μl | 624 ± 21 | 626 ± 36 | 0.959 |
| MPV | fL | 5.2 ± 0.1 | 5.5 ± 0.1 | 0.051 |

Red blood cells

| | | | | |
|---|---|---|---|---|
| RBC | 10⁶/μl | 7.6 ± 0.1 | 6.8 ± 0.4 | 0.098 |
| HGB | g/dL | 11.6 ± 0.2 | 10.6 ± 0.5 | 0.128 |
| HCT | % | 36.1 ± 0.4 | 33.2 ± 1.8 | 0.184 |
| MCV | fL | 48.0 ± 0.3 | 48.4 ± 0.3 | 0.341 |
| MCH | pg | 15.3 ± 0.1 | 15.5 ± 0.1 | 0.274 |
| MCHC | g/dL | 32.2 ± 0.1 | 31.9 ± 0.2 | 0.289 |
| RDW | % | 13.6 ± 0.1 | 12.9 ± 0.1 | 0.002 |

| | | |
|---|---|---|
| Reticulocytes | Negative | Negative |

White blood cells

| | | | | |
|---|---|---|---|---|
| WBC | 10³/μl | 1.25 ± 0.26 | 1.43 ± 0.22 | 0.609 |
| NE | 10³/μl | 0.01 ± 0.00 | 0.04 ± 0.02 | 0.184 |
| LY | 10³/μl | 1.23 ± 0.26 | 1.35 ± 0.21 | 0.719 |
| MO | 10³/μl | 0.01 ± 0.00 | 0.02 ± 0.00 | 0.387 |
| EO | 10³/μl | 0.00 ± 0.00 | 0.00 ± 0.00 | 0.408 |
| BA | 10³/μl | 0.00 ± 0.00 | 0.00 ± 0.00 | 0.245 |

Feces

| | | |
|---|---|---|
| Hemocult ® | Negative | Negative |

TABLE 4B

| | | | | |
|---|---|---|---|---|
| Parameter | Unit | Control (n = 10) | Acrp30g-2 (n = 10) | p |

Platelet Rich Plasma

| | | | | |
|---|---|---|---|---|
| Howell time | s | 195 ± 7 | 233 ± 9 | 0.01 |
| Platelet in PRP | 10³/μl | 619 ± 108 | 499 ± 81 | ns |

Blood parameter
Platelets

| | | | | |
|---|---|---|---|---|
| PLT | 10³/μl | 729 ± 22 | 719 ± 20 | 0.756 |
| MPV | fL | 5.3 ± 0.1 | 5.6 ± 0.1 | 0.037 |

Red blood cells

| | | | | |
|---|---|---|---|---|
| RBC | 10⁶/μl | 7.5 ± 0.2 | 7.3 ± 0.1 | 0.318 |
| HGB | g/dL | 11.7 ± 0.2 | 11.4 ± 0.1 | 0.290 |
| HCT | % | 36.8 ± 0.7 | 36.4 ± 0.4 | 0.614 |
| MCV | fL | 49.2 ± 0.4 | 49.9 ± 0.3 | 0.146 |
| MCH | pg | 15.6 ± 0.1 | 15.6 ± 0.1 | 0.917 |
| MCHC | g/dL | 31.7 ± 0.1 | 31.3 ± 0.1 | 0.003 |
| RDW | % | 12.2 ± 0.2 | 11.8 ± 0.2 | 0.090 |

| | | |
|---|---|---|
| Reticulocytes | Negative | Negative |

White blood cells

| | | | | |
|---|---|---|---|---|
| WBC | 10³/μl | 1.12 ± 0.14 | 1.16 ± 0.12 | 0.852 |
| NE | 10³/μl | 0.01 ± 0.00 | 0.01 ± 0.00 | 0.867 |
| LY | 10³/μl | 1.09 ± 0.13 | 1.12 ± 0.12 | 0.869 |
| MO | 10³/μl | 0.01 ± 0.00 | 0.01 ± 0.00 | 0.869 |
| EO | 10³/μl | 0.00 ± 0.00 | 0.00 ± 0.00 | 0.357 |
| BA | 10³/μl | 0.00 ± 0.00 | 0.00 ± 0.00 | 0.305 |

Feces

| | | |
|---|---|---|
| Hemocult ® | Negative | Negative |

The abbreviations used in Table 4 are as follows: PLT: platelets; MPV: mean platelet volume; RBC: red blood cells; HGB: hemoglobin; HCT: hematocrit; MCV: mean corpuscular volume; MCH: mean corpuscular hemoglobin; MCHC: mean corpuscular hemoglobin concentration; RDW: red cell distribution width; WBC: white blood cells; NE: neutrophils; LY: lymphocytes; MO: monocytes; EO: eosinophils; BA: basophils.

Conclusion

The Howell time was significantly increased by a daily treatment with Acrp30g-2 at 100 μg/kg. These results showed anti-aggregant and/or anti-coagulant properties of Acrp30g-2.

In addition, no hemorrhagic effect in the gastrointestinal tractus was observed. Blood hemoglobin, erythrocytes and hematocrit were not changed. Reticulocytes and blood were not detected in blood and feces, respectively. Only MPV and MCHC were weakly changed. These low changes do not have any pathophysiological significance. These results confirm that Famoxin should be devoid of prohemorrhagic properties.

### Example 4

### Antiagregggant/Anti-Coagulant and Prohemorrhagic Properties of a Repeated Treatment with Acrp30g-2 in C57BL/6 Mice

Material and Methods

Acrp30g-2 was administered to C57BL/6 mice at the dose of 100 μg/kg, twice daily during 5 days (9 administrations), by subcutaneous route. Two hours after the last injection the mice were exanguinated by intracardiac puncture under isoflurane anaesthesia. The blood was collected in vials containing citrate as anti-coagulant. Each experimental group had 6-8 mice. Anti-aggregant/anti-coagulant effects were assessed by the Howell time. PRP was obtained from citrated blood by centrifugation (250 g×10 min). The Howell time was determined as the time to get coagulation when 25 mM CaCl₂ (100 μl) was incubated with PRP (100 μl) at 37° C. Prohemorrhagic effects were assessed by the search of blood in feces (Hemocult®) and the determination of the red blood cell and platelet related parameters using a Beckman-Coulter counter.

Results

The results are shown in table 5. The Howell time was significantly increased by a daily treatment with AS902036 (100 μg/kg, sc). Acrp30g-2 decreased significantly the number of platelets in PRP, but not in whole blood. This suggests that Acrp30g-2 modifies the volume and/or density of platelets. In addition, these results showed that Acrp30g-2 exhibits anti-aggregant and/or anti-coagulant properties.

TABLE 5

| | | | | |
|---|---|---|---|---|
| Parameter | Unit | Control (n = 6) | Acrp30g-2 (n = 8) | p |

Platelet Rich Plasma

| | | | | |
|---|---|---|---|---|
| Howell time | sec | 125 ± 6 | 150 ± 6 | 0.018 |
| Platelet in PRP | 10³/μl | 661 ± 76 | 351 ± 62 | 0.008 |

Blood parameter
Platelets

| | | | | |
|---|---|---|---|---|
| PLT | 10³/μl | 575 ± 26 | 603 ± 26 | 0.477 |
| MPV | fL | 5.0 ± 0.1 | 5.0 ± 0.0 | 0.913 |

Red blood cells

| | | | | |
|---|---|---|---|---|
| RBC | 10⁶/μl | 6.98 ± 0.08 | 6.93 ± 0.16 | 0.778 |
| HGB | g/dL | 10.3 ± 0.1 | 10.1 ± 0.2 | 0.476 |
| HCT | % | 31.5 ± 0.3 | 31.4 ± 0.7 | 0.54 |
| MCV | fL | 45.2 ± 0.3 | 45.3 ± 0.6 | 0.685 |
| MCH | pg | 14.8 ± 0.0 | 14.6 ± 0.1 | 0.26 |
| MCHC | g/dL | 32.6 ± 0.2 | 32.1 ± 0.1 | 0.044 |
| RDW | % | 12.0 ± 0.1 | 11.9 ± 0.1 | 0.706 |

| | | |
|---|---|---|
| Reticulocytes | Negative | Negative |

White blood cells

| | | | | |
|---|---|---|---|---|
| WBC | 10³/μl | 1.3 ± 0.2 | 1.6 ± 0.2 | 0.349 |
| NE | 10³/μl | 0.0 ± 0.0 | 0.0 ± 0.0 | 0.972 |
| LY | 10³/μl | 1.2 ± 0.2 | 1.6 ± 0.2 | 0.319 |
| MO | 10³/μl | 0.0 ± 0.0 | 0.0 ± 0.0 | 0.206 |
| EO | 10³/μl | 0.0 ± 0.0 | 0.0 ± 0.0 | 0.234 |
| BA | 10³/μl | 0.0 ± 0.0 | 0.0 ± 0.0 | 0.234 |

Feces

| | | |
|---|---|---|
| Hemocult ® | Negative | Negative |

Conclusion

Examples 3 and 4 show that the treatment of normal and db/db mice with Acrp30g-2 induced a significant antiagregggant and/or anti-coagulant effect without modifying the platelet number, and without gastric prohemorrhagic effect.

### Example 5

### Effect of 2 Week Treatment with Acrp30g-2 on Tail Vein Bleeding Time in C57BL/6 Mice

Introduction—

To address whether could contrite to the regulation of hemostasis, the effect of chronic Acrp30g-2 injection on tail vein bleeding time in mice was tested. This parameter evaluates the capacity of platelets to form a plug in transversally cut small vein and arteries in vivo.

Material and Methods

Group 1 corresponded to obese mice. This group was comprised of 7 weeks old female C57BL/6 mice fed with a high-fat diet. After 5 month on this diet mice gained weight up to 38 to 42 g. A group of 6 mice was treated for 7 days with two injections per day of Acrp30g-2 at 50 μg/kg, followed by a 7 days treatment with three injections per day of Acrp30g-2 at 50 μg/kg. Acrp30g-2 was administered subcutaneously (+ on FIG. 3). The control group (n=6) was injected with equivalent volume of sterile physiological saline with the same schedule of injection (Obese − on FIG. 3). After 2 weeks of treatment the animal were briefly anesthetized with isoflurane and the tail was cut transversally with scalpel blade 2 mm from extremity. A timer was started at the time of tail cut and blood drops were recovered on whatman paper until bleeding ceased. The time in sec of occurrence of bleeding cessation was recorded for each mouse. The results are the mean+/−SEM of bleeding time expressed in second. Statistical significance of the difference between mean was determined by Student's t test.

Group 2 corresponded to lean mice. This group was comprised of 8 weeks old female C57/bl 6 mice (n=12) that were accustomed to high-fat diet for 7 days without other treatment. At the end of this, the animals were randomly split in 2 groups of 6. Acrp30g-2 treated group received (2*50 μg/kg per day SC) for 7 days then the dose was adjusted to 3*50 μg/kg per day and the treatment continued for 7 days (Lean + on FIG. 1). Saline injections of controls were adjusted to match volume and schedule of Sc injections (Lean − on FIG. 1). At the end of 2 week treatment mice were anesthetized with isoflurane and tested for tail bleeding time exactly as described above for the obese group.

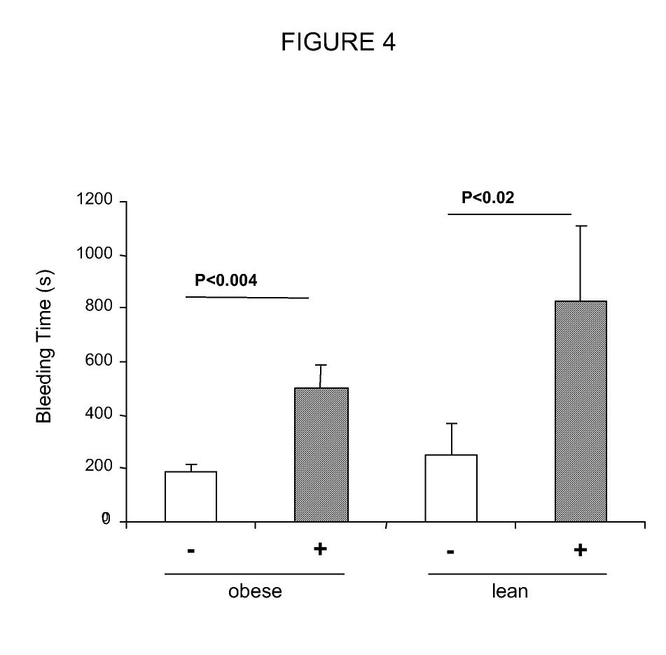

The results are shown on FIG. 4.

Conclusion

Daily injection of Acrp30g-2 over a 2 weak period significantly increased tail bleeding time in mice under high-fat diet. The increase in tail bleeding time induced by Acrp30g-2 treatment is significant in both lean and obese mice. No significant effect of high fat feeding on tail bleeding time is observed. Interestingly, tail bleeding time was measured only 12 h after the last Acrp30g-2 injection. This demonstrates an action of Acrp30g-2 on primary hemostasis.

### Example 6

### Effect of 2 Week Treatment with Acrp30g-2 on TCT, Platelets Count and Fibrinogen Level in C57BL/6 Mice

Introduction

The experiment below was performed to rule out the possibility that increased bleeding time observed in Example 4 resulted from reduction in platelets number.

Methods and Results

The animals used in the experiment of tail bleeding described in Example 5 were next tested for changes in main hematological parameters. To achieve this, within an hour of completing the tail bleeding time measurements, animals were anesthetized using isoflurane and blood samples were collected through carotid artery opening. Samples were collected into tubes containing citrate as an anticoagulant in order to allow measurement of platelet number, thrombin clotting time (TCT) and fibrinogen levels. All 3 parameters were determined using routine procedures of clinical hematology laboratories such as those disclosed, e.g., in the laboratory manual “Manuel d'hémostase” (J. Sampol, D. Arnoux and B. Boutière, Elsevier, 1995).

Results

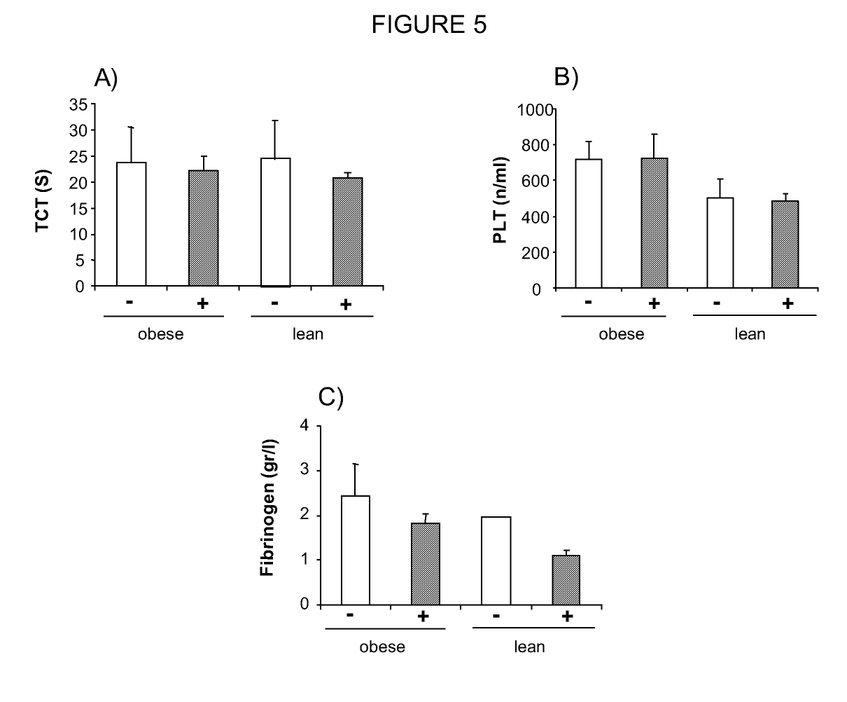

The results show that Acrp30g-2 chronic exposure had not detectable effect on TCT (FIG. 5A), which provides a measure of the efficacy of proteolytic cascades leading to production of fibrin. Platelets counts were not significantly decrease by chronic exposure to Acrp30g-2 for up to 2 weeks (FIG. 5B). No difference in plasma fibrinogen levels was observed in mice treated with Acrp30g-2 (FIG. 5C).

Conclusion

Experiments described in examples 5 and 6 indicate that chronic Acrp30g-2 treatment significantly increased tail bleeding time and that this effect was not due to platelet loss. Demonstration of the lack of effect on thrombin clotting time is consistent with Acrp30g-2 decreasing platelets capacity to form a clot, thereby delaying cessation of bleeding.

### Example 7

### Effect of Single Injection of Acrp30g-2 on Tail Bleeding Time in C57BL/6 Mice

Introduction

The experiment below was performed to determine whether the increased tail bleeding time observed after 2 weeks of chronic exposure to Acrp30g-2 also occurred after a single injection. It was further carried out to verify that the effect was not related to administration of the high-fat diet.

Methods

A group of 12 to 14 weeks old C57BL/6 female mice, fed with normocaloric diet, were injected with increasing doses of Acrp30g-2, administered subcutaneously, 3 hours prior to performing tail bleeding time experiments. This schedule of injection was selected on the basis of pharmacokinetics data obtained using radiolabelled Acrp30g-2. The pharmacokinetic data showed that highest plasma concentration of Acrp30g-2 was achieved between 3 and 4 hours flowing subcutaneous injection of Acrp30g-2. Animals were injected subcutaneously with the indicated dose of Acrp30g-2 at nine o'clock in the morning and maintained on regular light cycles. Tail bleeding time experiments were started 3 h after the injection of Acrp30g-2. The same protocol of isoflurane anaesthesia and transversal tail cutting described in example 5 was applied.

Results

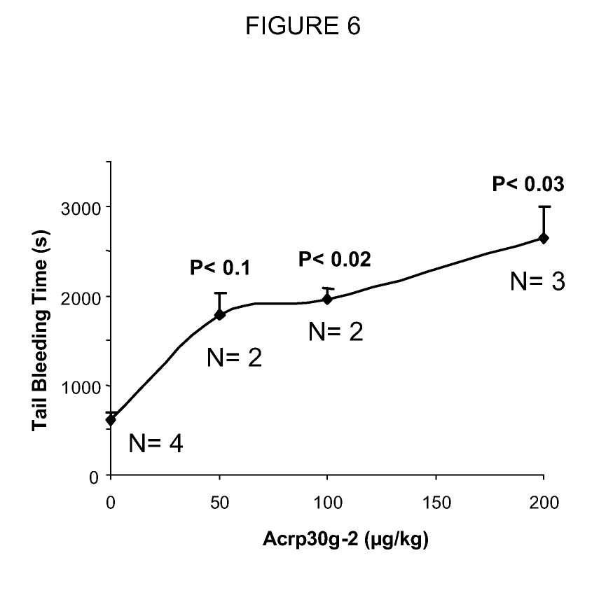

The results shown in FIG. 6 indicate that a single injection of Acrp30g-2, at the dose of 100 μg/kg of body weight, was sufficient to significantly increase tail bleeding time. The effect was stronger with a dose of 200 μg/kg. Injection of a dose of 50 μg/kg, although tending to increase tail bleeding time, did not increase this parameter in a statistically significant manner.

Conclusion.

A single injection of 100 μg/kg of Acrp30g-2 to normal mice under regular diet significantly prolonged the time needed for platelets to form a clot at the extremity of transversally cut arterial and venous vessels. This effect was detectable within 3 hours following a single subcutaneous injection of Acrp30g-2.

### Example 8

### In Vitro Effect of Acrp30g-2 on Platelet Aggregation

Introduction

Experiments 4 to 6 were performed in vivo. In the present experiment, Acrp30g-2 was added directly to platelets rich plasma obtained from mice under high-fat diet, and it was determined whether this caused delayed platelets aggregation.

Method

Mice fed with high-fat diet (n=12), and with mean body weight 27±3 grams, were anesthetized with isoflurane and blood was collected from carotid artery directly on citrate tube to prevent coagulation but also allow platelets aggregation after recalcination. Only the first 700 μl of blood were collected from each animal in order to minimize interference of clotting factors induced by carotid hemorrhage. The blood samples were then pooled and platelets rich plasma was obtained by 10 nm centrifugation at room temperature and 100 g. PRP was recovered and distributed to 8 glass tubes. 2 samples were then supplemented with Acrp30g-2 in order to achieve final concentration of 1 μg/ml. The 2 remaining samples were supplemented with equivalent volume of saline solution. Sixty seconds after addition of Acrp30g-2, the samples were supplemented with 22 mM Ca²⁺. The samples were then placed in a water bath at 37° C. and agitated with curved glass Pasteur pipette until a platelet clot was trapped by the Pasteur pipette. The time of occurrence of this event was recorded for each tube.

Results

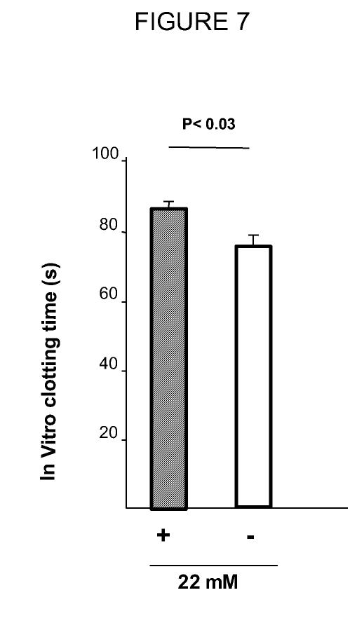

Acrp30g-2 significantly increased the clotting time measured in the presence of calcium (FIG. 7).

### Example 9

### Establishment of a Mouse Model for Acute Pulmonary Embolism

Introduction

A mouse model of massive intravenous platelets aggregation leading to pulmonary embolism and death was established, and a preliminary test was carried out to determine whether Acrp30g-2 exerts an antiagregggant effect in vivo.

Method

Female C57BL/6 mice were anesthetized with 60 mg/kg of pentobarbital. The tail vein was then cannulated and injected with collagen associated to 45 μg/kg of epinephrine. Collagen is known to induce platelet aggregation (Savage et al., 2001). A group of mice (n=9) received an injection of 500 μg of Acrp30g-2, 3 hours prior to establishing collagen dose response curve. In this experiment, collagen injection of 0.250 μg/kg and 0.375 μg/kg induced the death of a significant number of animals. The maximal death rate was 80%. The death occurred within 3 to 6 nm from collagen injection. Animals that (i) survived this critical period; and (ii) were alive 10 min post injection were considered as survivors. Scientific literature teaches that survival for a period greater that 10 nm indicates that a mouse has definitively overcome a collagen challenge (Angelillo-Scherrer et al., 2001). However, in order to avoid inflicting unnecessary pain to surviving animals, results of all experiments were analyzed as follows: animal still alive 10 nm after collagen injection were considered survivors, while those experiencing absence of breathing for more than 1 nm during the 10 nm after the collagen injection were classified as dead. All animals were subsequently sacrificed by cervical dislocation under pentobarbital anesthesia.

In a second set of experiment, mice were injected with 375 μg/kg collagen and 45 μg/kg epinephrine. Acrp30g-2 was injected 5 min after the collagen/epinephrine challenge.

Results

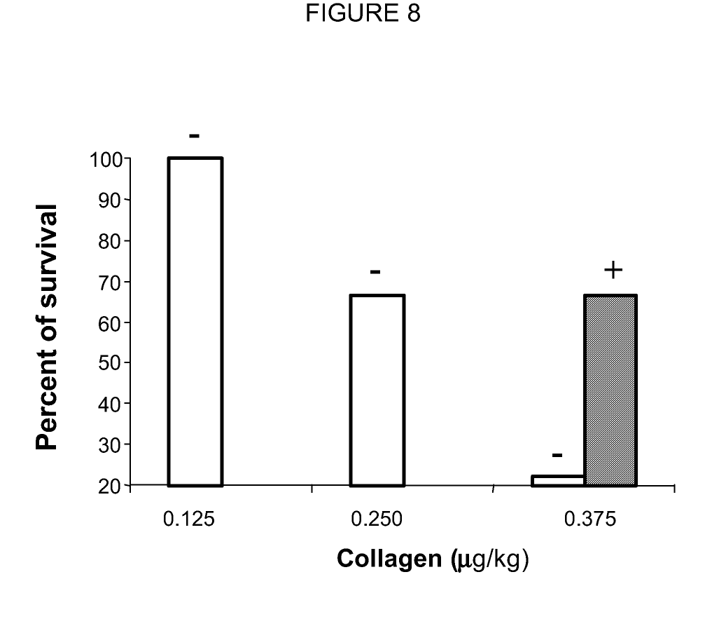

In the first set of experiments, the collagen injection in tail vein caused a death rate of about 80% in control mice. In mice treated with 500 μg/kg of Acrp30g-2 three hours prior to the collagen injection, six animals out of nine survived the collagen injection (FIG. 8).

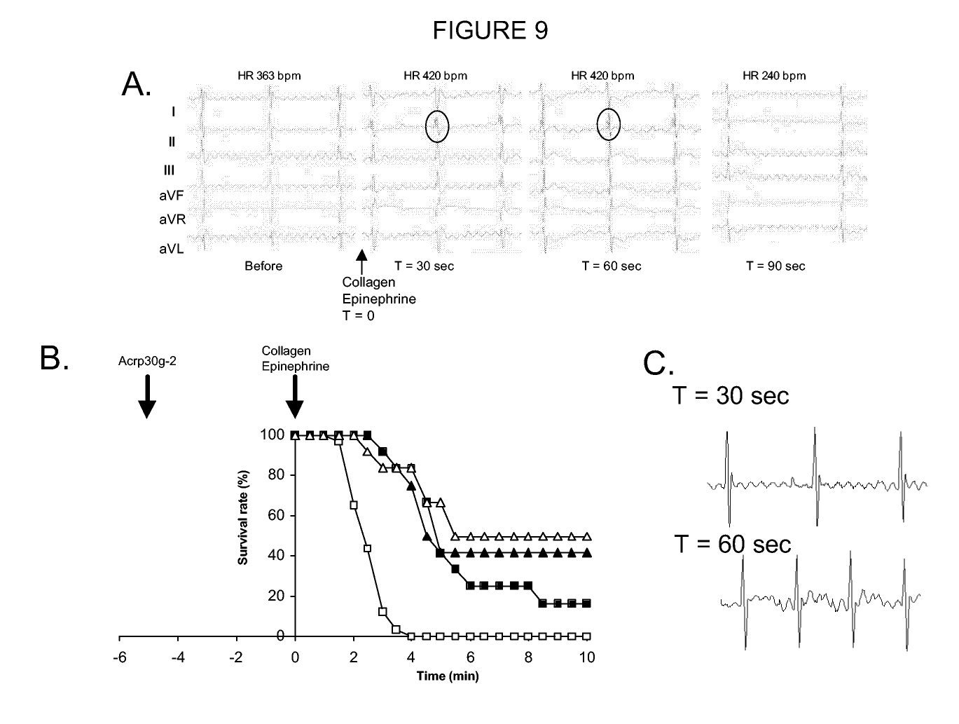

In the second set of experiments, 100% death occurred within 5 min. FIG. 9B shows that Acrp30g-2 significantly improved survival rate up to 50% in a dose dependant manner (FIG. 9B).

The occurrence of PE in this mouse model was documented using an electrocardiogram (ECG) system. As shown in FIG. 9A, mice injected with collagen and epinephrine through the tail vein experienced tachycardia, demonstrated a right shift of the cardiac axis and occurrences of r′ waves in lower derivations. Initial tachycardia was followed by bradycardia and gasping episode(s), respiratory arrest, and then death. Interestingly, ECG data obtained on survivors (FIG. 9C) did not exhibit the typical r′wave observed in second derivation of non survivors (FIG. 9A).

Conclusion

Regarding the mouse model, these results show that it was possible to create massive intravenous platelets aggregation with tail vein collagen injection. This effect was collagen-dose dependent. This mouse model for pulmonary embolism leads to a mortality of about 80% to 100%.

Regarding the in vivo effect of Acrp30g-2, it was demonstrated that injection of 500 μg/kg of Acrp30g-2 three hours before the collagen injection decreased mortality by about 40% to 50% in a mouse model for pulmonary embolism.

### Example 10

### Effect of Preventive Treatment with Acrp30g-2 on the Survival Rate of a Mouse Model for Acute Pulmonary Embolism

Introduction

This experiment was performed in order to test the statistical significance of the effect of Acrp30g-2 in the mouse model for pulmonary embolism described above.

Methods

Normal mice were challenged with collagen at 0.250 mg/kg and epinephrine at 30 μg/kg as described above. The control group was comprised of 21 mice. A second group, comprised of 12 mice, received a subcutaneous injection of 50 μg/kg of Acrp30g-2 three hours before being subjected to the collagen-epinephrine challenge. A third group, comprised of 12 mice, received a subcutaneous injection of 500 μg/kg of Acrp30g-2 three hours before being subjected to the collagen challenge.

Results

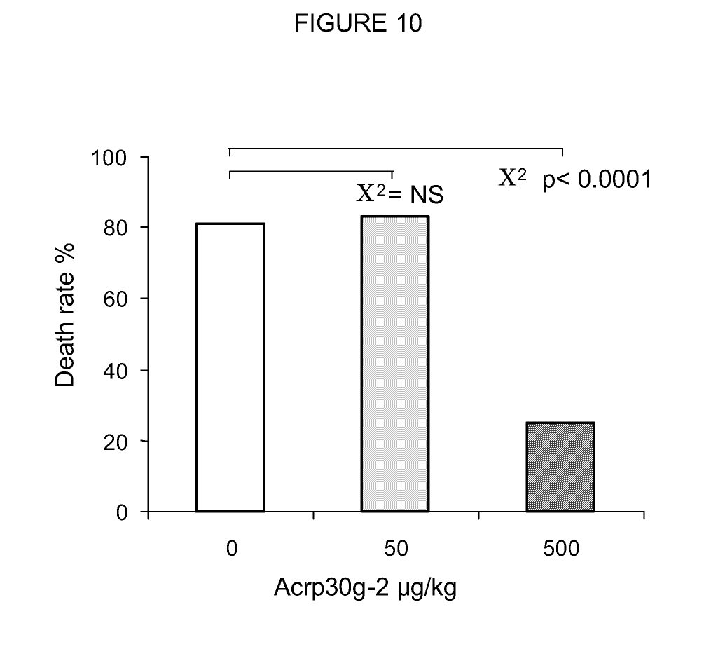

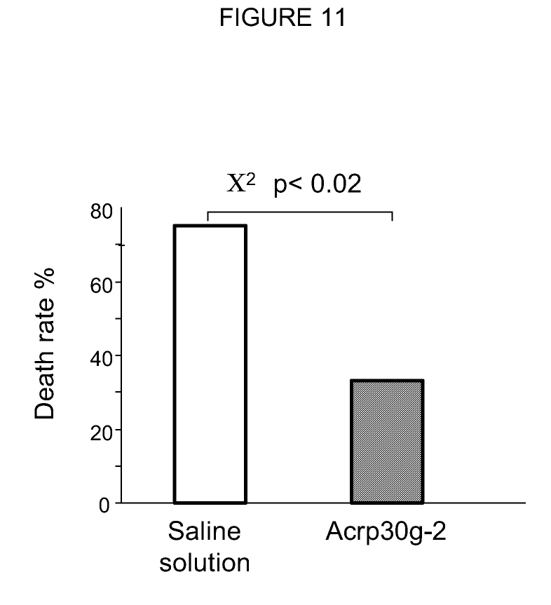

The mortality rate in the second group remained at 80%, i.e. the mortality rate was identical to that of the control group (FIG. 10). In the third group, the mortality rate fell to 25%. Statistical significance of the obtained data was tested using the Chi square analysis. No difference in death versus survivor frequency was found between the control group and the second group. The difference was statistically significant when comparing the third group and the control group.

Conclusion

Subcutaneous injection of Acrp30g-2 at 500 μg/kg three hours before collagen injection was able to dramatically reduce death resulting from massive pulmonary embolism subsequent to platelets aggregation induced by direct injection of collagen in the tail vein of mice. Acrp30g-2 therefore rapidly inhibits progressive platelets aggregation. As a consequence, Acrp30g-2 can be used for the treatment of acute pulmonary embolism that occurs following, e.g., deep vein thrombosis or atrial fibrillation.

### Example 11

### Effect of Injection of Increasing Doses of Acrp30g-2 on the Survival Rate

Introduction

On the basis of results described in Example 9 and 10, Acrp30g-2 can be used as a preventive measure of venous thrombosis complication. The present experiment aimed at determining whether Acrp30g-2 could be used as an emergency drug with the potential of stopping the progression of already initiated massive platelets aggregation leading to pulmonary embolism.

Method

Tail vein of normal mice were canullated and injected with 0.375 μg/kg of collagen and 45 μg/kg of epinephrine. The cannula remained in place and 30 s after collagen injection, and either 200 μg/kg of Acrp30g-2 or an equivalent volume of saline solution was injected intravenously. Twelve mice were injected with each solution. The Acrp30g-2 dose was chosen to achieve a plasma concentration ranging between 1 and 1.6 μg/ml based on extrapolated pharmacokinetic data.

Results

In the control group injected with saline solution, four mice out of twelve survived. In the group injected with Acrp30g-2, eight mice out of twelve survived (FIG. 10).

Conclusion:

Acute injection of Acrp30g-2 significantly reduced the mortality rate due to pulmonary embolism when Acrp30g-2 was administered 30 seconds after initiating massive platelets aggregation in the venous compartment of mice tail. These results indicate that the effect of Acrp30g-2 on rapidly aggregating platelets was sufficiently rapid and potent to provide a therapeutic for ongoing pulmonary embolism.

### Example 12

### Comparison of the Effect of Acrp30g-2 with the Effect of Heparin

Introduction

It was next sought to test the therapeutic potential of Acrp30g-2 by comparison with heparin.

Method

The collagen-epinephrine challenge was performed with 0.375 mg/kg of collagen and 45 μg/kg of epinephrine.

Two groups of animals was injected, 30 min prior to the collagen-epinephrine challenge, with Heparin at two different doses:
125 IU/kg, which corresponds to the maximal dose of 10,000 IU used under emergency conditions in human subjects; or
500 IU/kg.

A third group of animals was injected with Acrp30g-2 at 400 μg/kg.

A fourth group of animals were injected with both Acrp30g-2 and heparin.

Heparin was injected through the intraperitoneal route since heparin injections through the mouse tail vein rendered subsequent intraventricular collagen-epinephrine injections impossible. In contrast, intraventricular injections of even very high doses of Acrp30g-2 did not modify tail vein appearance and thus did not interfere with subsequent collagen-epinephrine injections.

Results

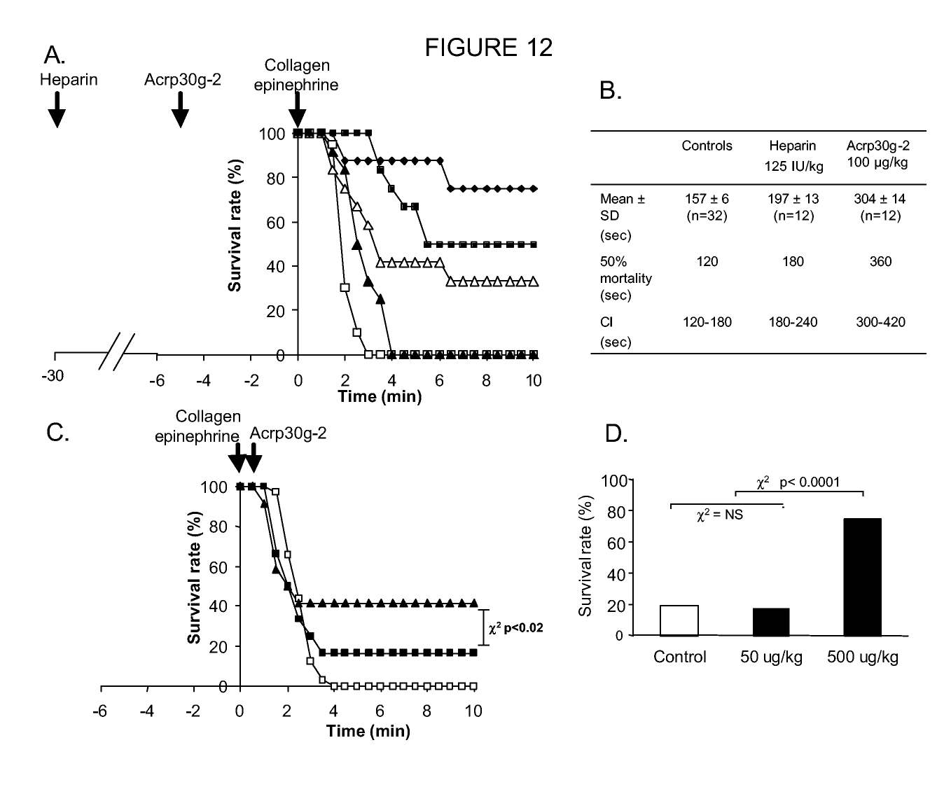

Heparin was an effective treatment of PE in mice at 500 IU/kg, and improved survival rate by 40%. Under these same conditions, Acrp30g-2 increased survival by 50%. Injection of Acrp30g-2 and heparin, each at the maximal dose, led to an additive effect. Indeed, the survival rate was of about 80% when both Acrp30g-2 and heparin were injected (FIG. 12A). In order to better evaluate the therapeutic potential of Acrp30g-2 relative to heparin, Kaplan-Meier analysis was applied to estimate survival time and the time leading to 50% mortality in animal treated with either the low dose heparin (125 IU/kg) or the low dose of Acrp30g-2 (100 μg/kg) (FIG. 12B).

Conclusion

These results indicate that heparin and Acrp30g-2 are effective therapeutic measures for the treatment of thromboembolic diseases. In addition, Acrp30g-2 is significantly more potent that heparin. Moreover, a cumulative effect is observed when both Acrp30g-2 and heparin are injected.

### Example 13

### Injection of Acrp30g-2 after the Collagen-Epinephrin Challenge

Introduction

It was further determined whether injecting Acrp30g-2 60 sec after the collagen-epinephrine challenge, i.e., when the animals experienced severe right ventricular after-load, still allows an increase in the survival rate.

Methods

The experiment was performed as described in the legend to FIG. 12.

Results

FIG. 12C shows that this is indeed the case. Although the efficacy appeared somewhat lower in comparison to injections performed 5 min prior to the challenge, the increased survival rate was statistically significant (p<0.02).

In addition, it was tested whether Acrp30g-2 administered subcutaneously (sc) rather than intravenously, 3 hours before the collagen-epinephrine challenge, was effective as a preventive measure. Based on pharmacokinetic data, it was estimated that the effective plasma concentration of Acrp30g-2 ranged between 800 and 1600 ng of Acrp30g-2 per ml of plasma (data not shown). It was then calculated that a single injection of Acrp30g-2 at 500 μg/kg, administered subcutaneously 3 hours prior to the collagen-epinephrine challenge, would increase plasma levels to this therapeutic range.

The results of FIG. 12D showed that Acrp30g-2 at 500 μg/kg, administered subcutaneously, significantly increased survival rate when compared to mice receiving injected either with a 10-fold lower dose of Acrp30g-2, or with a saline solution.

### Example 14

### Effect of Acrp30g-2 on Thrombin-Initiated Fibrin Formation

Introduction

Heparin's inhibitory effect on thrombin cleavage of fibrinogen is well established. To determine if Acrp30g-2 also acted on thrombin cleavage of fibrinogen, thrombin activity was studied in the absence or in the presence of Acrp30g-2.

Methods

Fibrin formation was measured as described in Tran and Stewart (2003).

Results

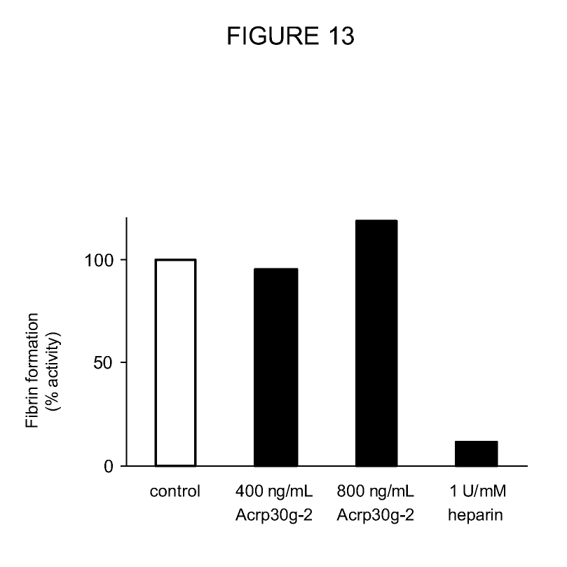

The results are shown in FIG. 13. Acrp30g-2 had no inhibitory effect on thrombin-induced fibrin formation. This suggests that heparin and Acrp30g-2 have different mechanisms of action, and act on different targets of the coagulation cascade. This result is consistent with the cumulative effect of heparin and Acrp30g-2 seen in Example 11.

### Example 15

### Effect of Acrp30g-2 on Platelets Activated by Collagen and Epinephrine, Thrombin, or ADP

Introduction

The effect of Acrp30g-2 on platelet aggregation was measured in vitro.

Methods

Blood from healthy volunteers was drawn and collected into tubes containing citrate (Becton Dickinson). Platelet rich plasma (PRP) was prepared immediately after collection by centrifugation at 200×g for 10 min at room temperature. Platelet poor plasma (PPP) was obtained by subsequent centrifugation at 1000×g for 10 min at room temperature. PRP and PPP were stored at room temperature and used within 1 hour after preparation. Platelet aggregation was measured at room temperature using an ELISA plate reader (BIORAD Benchmark Plus microplate spectrophotometer, reference No. 170-6935). Aliquots of 100 μL of PRP or of PPP per well were incubated in the absence or in the presence of Acrp30g-2. In some experiments, an agent inducing platelet aggregation was added. After addition of the agent inducing platelet aggregation, the plate was immediately placed in the plate reader, mixed for 20 sec, followed by a first reading 1 minute after addition of the agonist at 595 nm. Readings were obtained every minute; the plates were mixed 20 sec before each reading. % Transmittance values were calculated from the absorbance values, and PPP alone was considered as the reference value for 100% aggregation. In experiments using thrombin as an agent inducing platelet aggregation, platelets were pelleted and washed from PRP (method reference). Platelets were pelleted at 2000×g at room temperature, resuspended gently in Ca²⁺-free Tyrode's buffer containing 5 nM prostacyclin and re-pelleted. After a second washing, platelets were resuspended in Tyrode's buffer containing 2 mM CaCl₂ and no prostacyclin. The platelets were counted and the cell suspension adjusted to 1×10⁸ cells/ml. Platelet aggregation was measured in aliquots of 100 μL/well incubated in the absence or presence of the indicated concentrations of Acrp30g-2 and thrombin.

Results

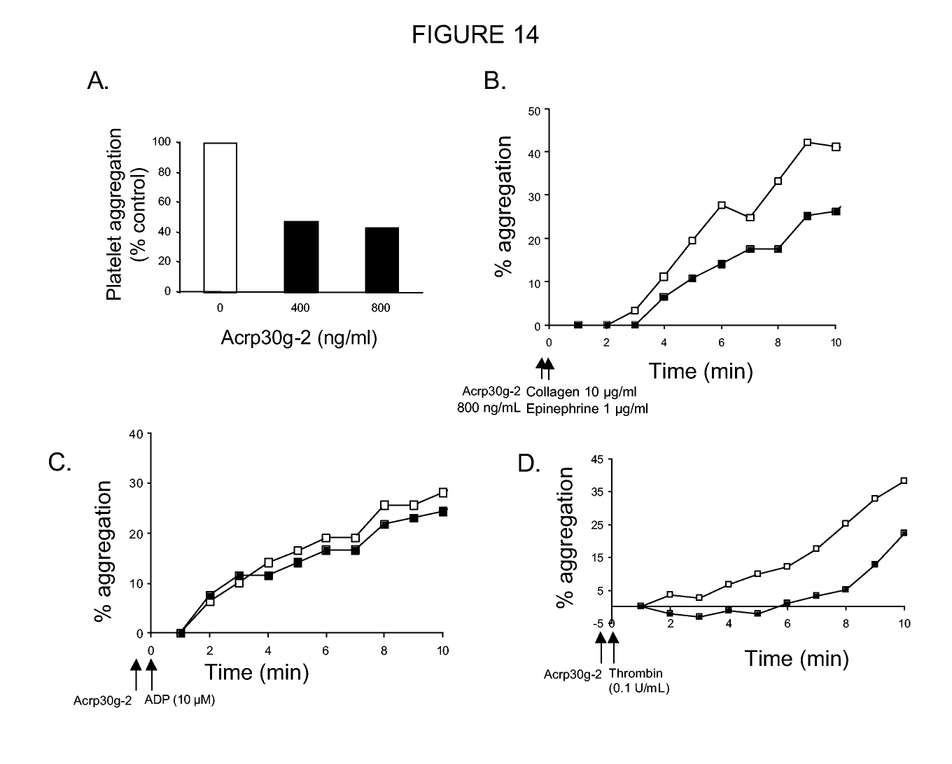

FIG. 14A shows that collagen-epinephrine induced platelet aggregation in mouse platelet rich plasma was inhibited by more than 50% in the presence of Acrp30g-2.

The present experiment also determined whether this inhibition of platelet aggregation induced by collagen-epinephrine was observed using human platelets. A representative experiment shown in FIG. 14B demonstrated that aggregation of human platelets induced by collagen and epinephrine is inhibited by Acrp30g-2 at concentrations that are in the same range as the concentrations leading to an increased survival rate in the mouse model for PE.

Human platelets aggregation induced by ADP was not affected by addition of Acrp30g-2 (FIG. 14C).

Thrombin is the most potent agent inducing platelet aggregation. A significant inhibition of thrombin-induced aggregation of washed human platelets incubated in presence of Acrp30g-2 was observed (FIG. 14D).

Conclusion

This experiment demonstrated that Acrp30g-2 exhibits anti-thrombin properties, and inhibits thrombin-induced platelet aggregation.

### Example 16

### Comparison of the Effect of Acrp30g-2 and of Full-Length Acrp30 on Platelets Activated by Thrombin

Introduction

The effect of Acrp30g-2 on platelet aggregation was measured in vitro was compared to the effect of full-length Acrp30.

Methods

The experiment was performed as described in example 15.

Results

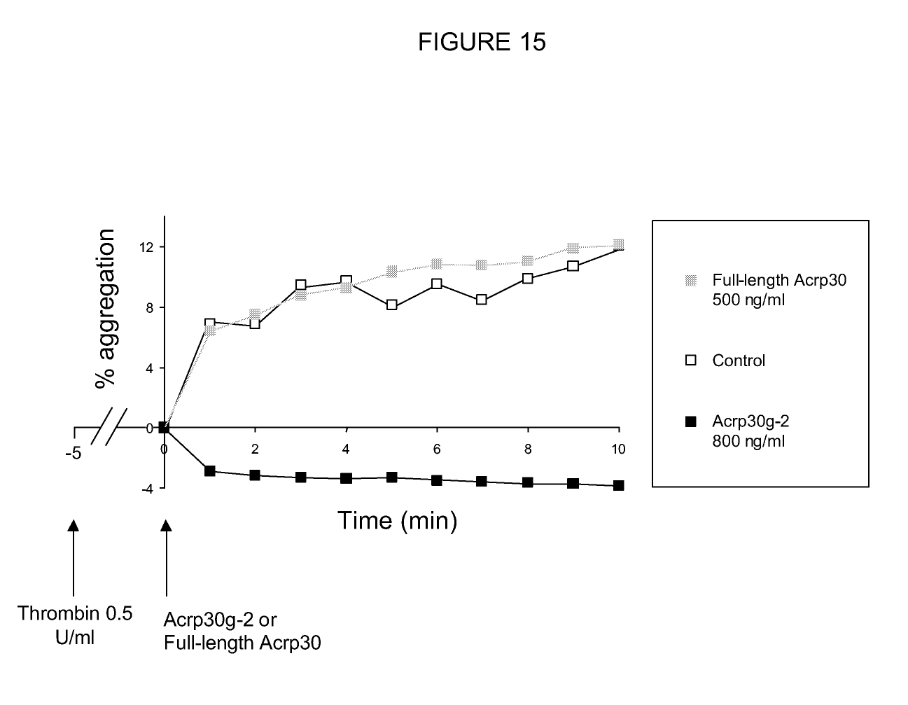

Full-length Acrp30 was completely ineffective at preventing collagen-epinephrine induced platelet aggregation (FIG. 15). Thus full-length Acrp30 did not display any inhibitory or desegregating properties on platelets in the presence of thrombin.

Conclusion

Acrp30g-2, but not full-length Acrp30, exhibits desegregating properties on platelets in the presence of thrombin.

### Example 17

### Effect of Acrp30g-2 on Platelets Activated by Thrombin

Introduction

Further experiments were performed to study the effect of Acrp30g-2 on thrombin-induced platelet aggregation.

Methods

The experiment was performed as described in example 15.

Results

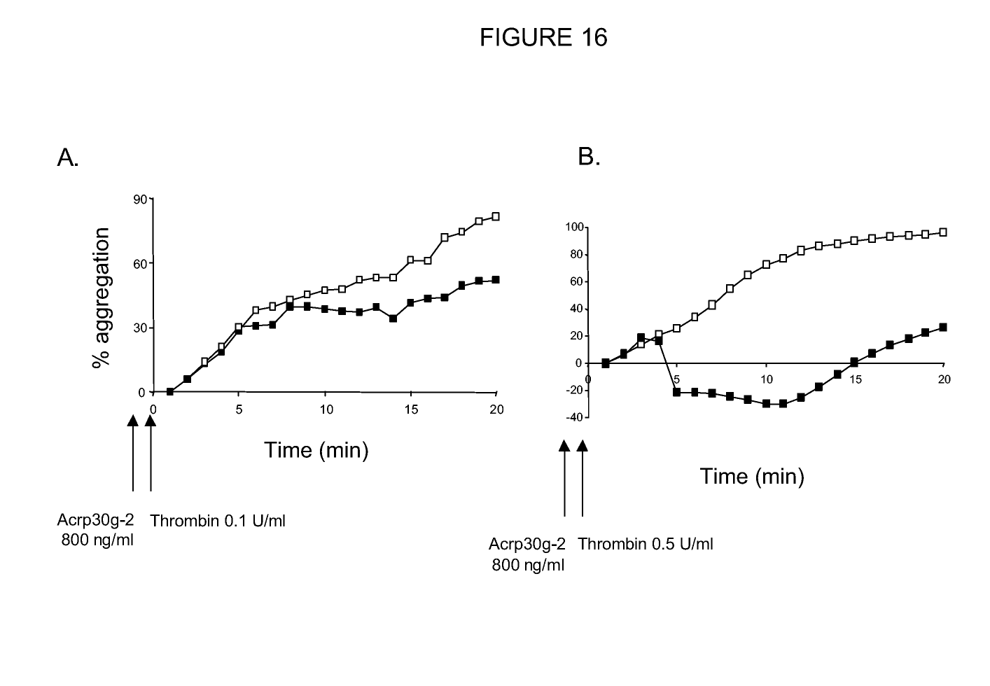

It was shown that Acrp30g-2 inhibits platelet aggregation induced either by 0.1 U/ml or 0.5 U/ml of thrombin (FIG. 16). At the lower concentration of thrombin, in the presence of Acrp30g-2, the platelets displayed a significant deceleration of platelet aggregation 5 min after addition of thrombin. At the higher concentration of thrombin, Acrp30g-2 displayed a strong disaggregation effect 4-5 min after thrombin induction.

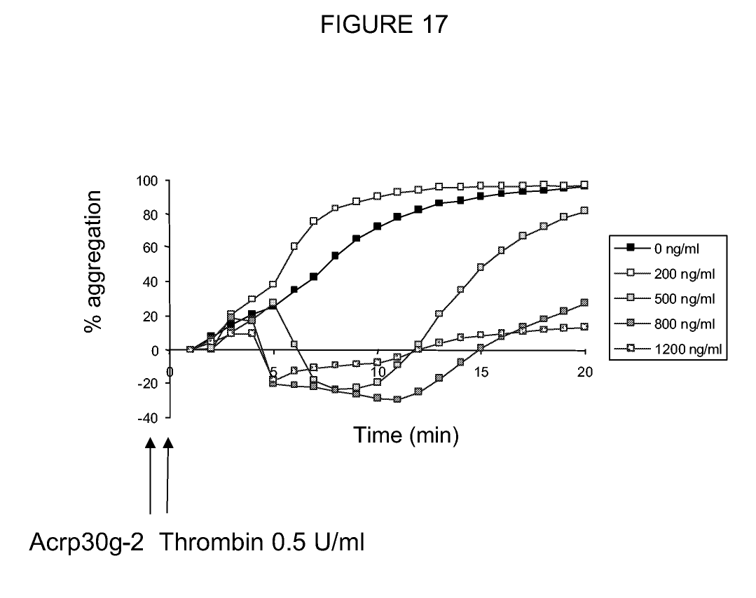

Dose response curves of Acrp30g-2 using platelets treated with thrombin demonstrated that a significant disaggregation effect is observed from 400 ng/ml to 1200 ng/ml of Acrp30g-2 (FIG. 17).

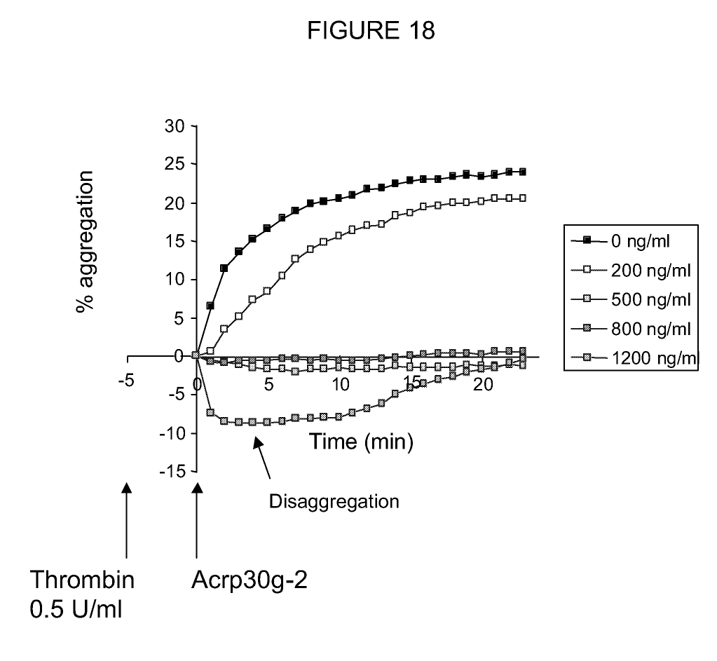

If Acrp30g-2 is added 5 min after inducing aggregation with thrombin, a significant inhibitory effect is observed in a dose-dependent manner (FIG. 18). Acrp30g-2 at 500 ng/ml completely stopped platelet aggregation, and disaggregation was observed with Acrp30g-2 at 1200 ng/ml.

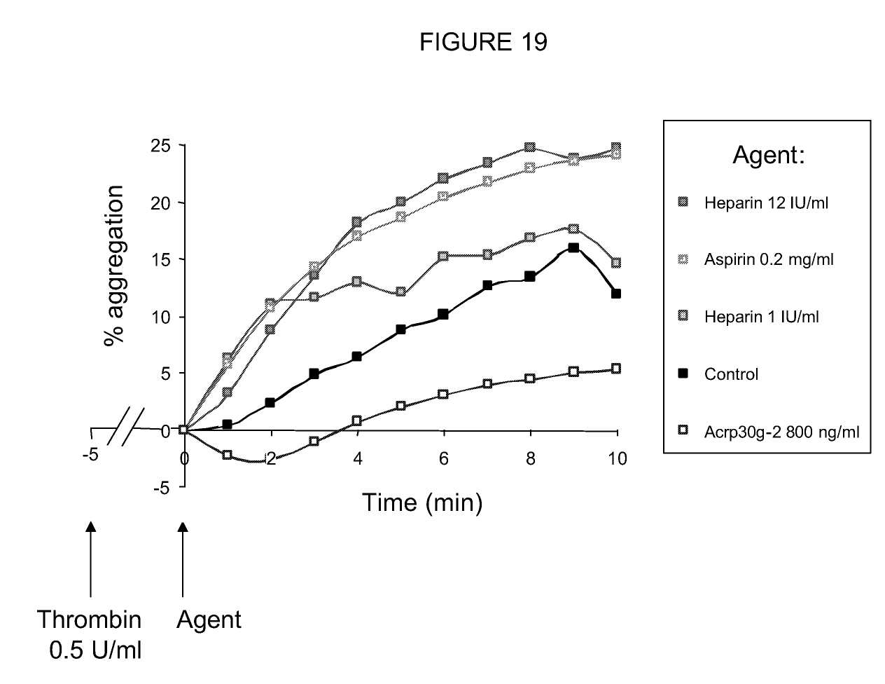

It was shown that, whereas Acrp30g-2 causes a significant disaggregating of platelets in the presence of thrombin, neither heparin nor aspirin exhibit a similar effect (FIG. 19). To the contrary, an increased platelet aggregation was observed at high doses of heparin or aspirin. It should be noted that there was no difference in the aggregation rate of the different samples prior to the addition of the pharmacological agents.

Conclusion

Acrp30g-2 exhibits a potent anti-aggregant activity, and an even a potent disaggregant activity on platelets in the presence of thrombin. Indeed, Acrp30g-2 causes desaggregation of human platelet activated by thrombin; neither heparin nor aspirin show any activity in this model. This further confirms that heparin and Acrp30g-2 have different mechanisms of action, and act on different targets of the coagulation cascade.

### Example 18

### Immunological methods

Sample Collection from Human Subjects.

Human blood samples were obtained from normal healthy volunteers by venous puncture. Blood was collected directly into dry tubes for serum or tubes containing EDTA or citrate. Samples for plasma preparations were placed on ice, and immediately centrifuged 1000× g for 20 min at 4° C. Serum was obtained after 30 min incubation at 37° C., followed by centrifugation under the same conditions as that for plasma.

Immunoprecipitation.

Immunoprecipitation was performed typically on 1 mL of fresh human plasma using an affinity purified polyclonal antibody referred to as AbAcrp30g. This antibody was produced in rabbit immunized by a recombinant protein containing a human Acrp30 sequence spanning from amino acids 110 to 244 of SEQ ID NO: 1. Immunoglobulins were purified using affinity chromatography on protein A followed by an affinity chromatography column using a recombinant protein (amino acids 110 to 244 of SEQ ID NO: 1) to capture conformation dependent antibodies. After several washes, protein were eluted from protein A and separated by SDS PAGE, transferred to PVDF membrane. The western blot was revealed with a biotinylated antibody directed to the globular head of human Acrp30 (Peprotech, Inc) or by a polyclonal antibody directed against the collagen tail. This antibody directed against the collagen tail was produced in rabbit immunized with a peptide located in the collagen tail (ETTTQGPGVLLPLPKGAC, which corresponds to amino acids 19 to 36 of SEQ ID NO: 1).

Native Molecular Mass Determination.

Native molecular mass determination for Acrp30g-2 was performed by gel filtration using an Akta Explorer 10 chromatography system and a Superdex 200 HR10-30 column (GE-Healthcare) equilibrated with PBS buffer (30 mM Sodium Phosphate, 150 mM NaCl, pH 7.4), at a flow rate of 0.5 mL/min. For calibration, the following molecular mass standards (Sigma) were used: (1) cytochrome c (12.4 kDa), (2) myoglobin (17 kDa), (3) carbonic anhydrase (29 kDa) and (4) bovine serum albumin (66 kDa). The void and total volumes of the column, 8.15 and 23.8 mL, respectively, were determined with potassium bichromate and blue dextran dyes to enable calculation of the distribution coefficient Kav.

Surface-Enhanced Laser Desorption Ionization Time of Flight Mass Spectrometry (SELDI-TOF)

Affinity purified AbAcrp30g (0.4 μg in PBS) were covalently immobilized on pre-activated RS100 ProteinChip® Arrays (Ciphergen Biosystems Inc., USA). The RS100 array consists of a surface with carbonyl diimidazole groups that dock proteins by covalently reacting with their NH₂ groups (N-terminal and Lysines). the arrays were incubated in the presence of antibodies 1 h at 25° C. in a humidity chamber and the residual active sites were blocked with 5 μl Blocking buffer (0.5 M ethanolamine pH 8.0) for 20 min. The arrays were then washed three times in the Bioprocessor (Ciphergen Biosystems Inc) with Washing buffer (100 mM sodium phosphate, 150 mM NaCl, Triton 0.5%, pH 7.4) and PBS. Acrp30g-2 (10 μg/mL) was spiked in human blood and coagulation was either allowed (serum) or prevented (plasma). A control experiment was performed by spiking Acrp30g-2 in serum (after coagulation). 50 μl of plasma (serum) and 50 μl of Binding buffer (100 mM sodium phosphate, 150 mM NaCl, Triton 0.1%, pH 7.4) were applied on each spot and incubated 1 h at 25° C. in a humidity chamber. Samples were then washed with 100 μl Binding buffer (3 times), PBS (3 times) and 5 mM HEPES pH 7 (1 time). The air-dried arrays were saturated with sinapinic acid in 0.1% trifluoroacetic acid and 50% acetonitrile before being read on the instrument (Ciphergen Protein Chip System, PCS 4000). The instrument settings were the followings: laser intensity 5000, focus mass 16000, molecular mass range 0 to 200 kDa. Hirudin (BHVK, 7034 Da), Cytochrome c (bovine, 12230 Da), Myoglobin (equine, 16951 Da), Carbonic anhydrase (bovine RBC, 29023 Da), Enolase (S. cerevisiae, 46671 Da), albumin (bovine serum, 66433 Da) and IgG (bovine, 147300 Da) were used as calibrators.

### Example 19

### Identification of Acrp30 Cleavage Products in Human Plasma

To screen for the presence of physiological Acrp30 cleavage products, human plasma was immunoprecipitated using an antibody that detects an epitope located within the globular head of the protein. This was followed by Western blotting using an antibody directed toward the globular head. Immunoprecipitations (IP) were performed on samples collected from healthy, normal volunteers. The AbAcrp30g polyclonal antibody and a commercially available monoclonal antibody detecting an epitope located within the globular head of Acrp30 (Preprotech) were used.

The presence of two Acrp30 physiological cleavage products was demonstrated: a 20 kDa band and a 15.4 kDa band that migrated at the same level as Acrp30g-2 (data not shown).

### Example 20

### Characterisation of the 20 kDa Acrp30 Cleavage Product

A proteolytic inhibitor cocktail was added to the plasma in order to verify that the 20 kDa band is not due to proteolysis occurring in the tube during IP procedure. No difference was found (data not shown). To rule out the possibility that detection of the 20 kDa band was due to contamination by immunoglobulin fragments, Western blots were revealed using anti-mouse IgG, Preprotech's monoclonal antibody directed to the globular heaf of Acrp30 and anti-human IgG. The results clearly establish that the 20 kDa band matched neither with IgG light chain deriving from human plasma nor with the antibodies used for the immunoprecipitation (data not shown). Thus this 20 kDa band corresponds to an Acrp30 cleavage product.

In order to screen our collection of human samples for the presence or the absence of the 20 kDa Acrp30 cleavage product, and considering that our plasma samples are stored frozen, it was tested whether freezing affects detectabilty of the protein after IP. It was shown that a freezing-thawing cycle did not change detection of the 20 kDa band in plasma (data not shown).

A systematic IP was performed on 29 obese individuals and on 30 lean individuals. The 20 kDa band was detected in some, but not all subjects. Full-length Acrp30 was always detected. The population of obese and lean subjects was then stratified for the presence and absence of the protein. The investigator determining the presence or absence of the 20 kDa Acrp30 cleavage product was blinded to the obese versus lean status of the individual. Analysis of the distribution of individuals positive and negative for the presence of the 20 kDa Acrp30 cleavage product in lean and obese populations was performed using Chi-square analysis. The results are shown in Table 5.

TABLE 5

| | Obese | Lean |
|---|---|---|
| Presence of the 20 kDa Acrp30 cleavage product | 9 | 17 |
| Absence of the 20 kDa Acrp30 cleavage product | 20 | 13 |

The 20 kDa Acrp30 cleavage product was detected in 57% of the lean individuals, and 31% of the obese individuals. These results show that a significantly greater proportion of obese subjects were defective for 20 kDa protein than the lean population (Table 5, χ², p<0.05).

The phenotype of obese and lean individuals were analyzed in function of the presence or absence of the 20 kDa band. The results for women are shown as Table 6. The results for men are shown as Table 7. Results expressed as Mean±SEM.

TABLE 6

| | Obese | | | Lean | | |
|---|---|---|---|---|---|---|
| | 20 kDa (+) | 20 kDa (−) | Student p | 20 kDa (+) | 20 kDa (−) | Student p |
| n | 5 | 10 | | 9 | 6 | |
| Age (years) | 30 ± 5 | 38 ± 3 | NS | 35 ± 2 | 32 ± 3 | NS |
| BMI (kg/m²) | 37.2 ± 1.1 | 36.8 ± 0.8 | NS | 20.4 ± 0.4 | 20.3 ± 0.4 | NS |
| Fat mass (kg) | 48.4 ± 3.8 | 48.1 ± 1.9 | NS | 14.1 ± 1.0 | 13.6 ± 1.2 | NS |
| Adiponectin (μg/ml) | 8.0 ± 1.3 | 7.3 ± 1.1 | NS | 9.1 ± 0.7 | 7.1 ± 1.5 | NS |
| Leptin (ng/ml) | 30.3 ± 5.2 | 43.3 ± 5.9 | NS | 11.0 ± 2.3 | 7.4 ± 1.3 | NS |
| Insulin (μU/ml) | 16.3 ± 4.3 | 12.2 ± 1.3 | NS | 5.5 ± 1.0 | 4.6 ± 0.7 | NS |
| Glycemia (g/l) | 0.94 ± 0.03 | 0.97 ± 0.03 | NS | 0.79 ± 0.02 | 0.81 ± 0.03 | NS |
| FFA (μM) | 487 ± 112 | 673 ± 39 | 0.036 | 380 ± 50 | 609 ± 61 | 0.007 |
| REE (Kcal) | 3284 ± 117 | 2960 ± 127 | 0.042 | 2410 ± 20 | 2345 ± 27 | 0.04 |

TABLE 7

| | Obese | | | Lean | | |
|---|---|---|---|---|---|---|
| | 20 kDa (+) | 20 kDa (−) | Student p | 20 kDa (+) | 20 kDa (−) | Student p |
| n | 4 | 10 | | 8 | 7 | |
| Age (years) | 32 ± 3 | 37 ± 3 | NS | 30 ± 3 | 33 ± 3 | NS |
| BMI (kg/m²) | 36.4 ± 1.7 | 34.7 ± 0.9 | NS | 22.2 ± 0.6 | 22.2 ± 0.6 | NS |
| Fat mass (kg) | 41.6 ± 3.7 | 38.2 ± 1.4 | NS | 10.4 ± 1.5 | 11.6 ± 1.6 | NS |
| Adiponectin (μg/ml) | 4.2 ± 0.8 | 3.5 ± 0.8 | NS | 5.6 ± 1.2 | 4.6 ± 1.0 | NS |
| Leptin (ng/ml) | 23.5 ± 5.5 | 14.4 ± 1.8 | NS | 3.6 ± 1.0 | 3.0 ± 0.5 | NS |
| Insulin (μg/ml) | 22.2 ± 7.7 | 15.1 ± 2.2 | NS | 5.9 ± 0.8 | 6.4 ± 1.0 | NS |
| Glycemia (g/l) | 0.97 ± 0.03 | 1.12 ± 0.07 | NS | 0.92 ± 0.02 | 0.92 ± 0.04 | NS |
| FFA (μM) | 469 ± 39 | 606 ± 51 | 0.055 | 390 ± 66 | 372 ± 69 | NS |
| REE (Kcal) | 4816 ± 246 | 3914 ± 235 | 0.014 | 3496 ± 51 | 3639 ± 93 | NS |

In women, the lack of detectable 20 kDa protein was associated with significantly higher plasma free fatty acid (FFA) level and significantly lower resting energy expenditure (REE). This was verified both in lean and in obese women groups. In men, obese subjects have higher FFA levels and a significantly lower REE. No differences were observed in the lean male group.

Conclusion

The 20 kDa cleavage product is present under physiological conditions in human plasma. Further, the protein is detectable in significantly lower proportions of obese subjects. The lack of the protein in plasma derived from obese subjects is associated with significantly lower REE and higher plasma FFA level.

### Example 21

### Characterisation of the 15.4 kDa Acrp30 Cleavage Product

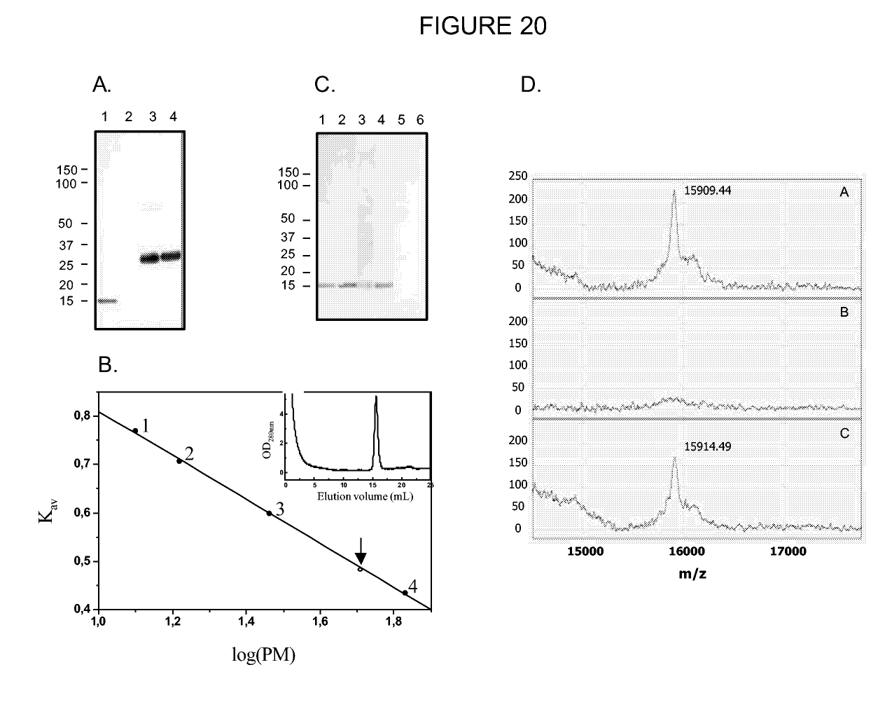

Immunoprecipitation (IP) were performed on normal human plasma using the AbAcrp30g polyclonal antibody, which is specifically directed against the globular head of human Acrp30, Subsequent Western blotting using either a commercially available antibody directed toward the globular head of Acrp30. or a polyclonal antibody directed toward the collagen tail of Acrp30 were performed. AbAcrp30g revealed a 15.4 kDa band corresponding to a protein containing the Acrp30 globular head (FIG. 20A, lane 1). No band corresponding to a protein containing the collagen tail was detected with polyclonal antibody directed toward the collagen tail of Acrp30 (FIG. 20A, lane 2). Positive controls showed that both the anti-globular head antibody and the anti-collagen tail antibody used in the Western blotting recognize full-length Acrp30 (data not shown). Thus:
(i) human plasma comprises an Acrp30 cleavage product of 15.4 kDa that comprises the globular head but not the collagen tail of Acrp30. This cleavage product is further referred to as Acrp30-15.4 kDa; and
(ii) The AbAcrp30g does not recognize the full-length protein.

It was further shown that AbAcrp30g is strictly conformation dependant. AbAcrp30g binds Acrp30-15.4 kDa in solution in plasma, but does not bind linear epitopes of the Acrp30-15.4 kDa protein on Western blot (data not shown). In addition, AbAcrp30g did not co-precipitate detectable amounts of full-length Acrp30, indicating that Acrp30-15.4 kDa is not bound to complexes containing full-length Acrp30.

All the above experiments were conducted using fresh plasma samples prepared from blood collected from normal healthy volunteers (n=4) into EDTA. Blood was collected into EDTA tubes, proteolytic inhibitor cocktail was added and after centrifugation, the plasma samples were placed at 4° C. and maintained at this temperature throughout the experiments. Control experiments using recombinant human full-length Acrp30 produced in eukaryotic cells showed that the protein spontaneously forms multimeric complexes and undergoes proteolytic cleavage at 37° C. Maintaining the samples at 4° C. or adding a protease inhibitor cocktail suppressed the proteolysis of full length Acrp30 (data not shown). This confirms that the Acrp30-15.4 kDa protein detected by the AbAcrp30g antibody is indeed present under physiological conditions in human plasma.

In the course of these studies, it was noticed that Acrp30-15.4 kDa was not detectable after IP performed on serum samples (data not shown). To test whether the coagulation process modifies the conformation of Acrp30-15.4 kDa, IP was performed on serum and plasma obtained from the same individual followed by a Western blot using the AbAcrp30g antibody. The results of FIG. 1b show that the coagulation process markedly decreased Acrp30-15.4 kDa recognition by the specific conformation dependant AbAcrp30g antibody.

Comparison of the relative size between Acrp30-15.4 kDa and full-length Acrp30, together with size prediction based on amino acid sequence led to the conclusion that the cleavage occurred at the alanine at position 108 of SEQ ID NO: 1. Acrp30-15.4 kDa thus corresponds to a cleavage product of Acrp30 consisting of amino acids 108 to 244 of SEQ ID NO: 1, i.e., to a polypeptide of SEQ ID NO: 3.

### Example 22

### Characterization of a Recombinant Acrp30g-2

A recombinant polypeptide of SEQ ID NO:3, referred to as Acrp30g-2, was produced in E. coli. After purification to homogeneity and refolding, Acrp30g-2 assembled as a stable trimeric structure with an apparent molecular mass of 47.7 kDa (FIG. 20B). Under denaturing gel electrophoresis, Acrp30g-2 migrated to a position identical to that of the Acrp30-15.4 kDa protein isolated from human plasma (FIG. 20C, lanes 1,2). In solution, the soluble Acrp30g-2 (FIG. 20D, lane 4) was precipitated by the conformation dependant AbAcrp30g antibody that selectively precipitates Acrp30-15.4 kDa from normal human plasma (FIG. 20C, lanes 1,3). Similarly to Acrp30-15.4 kDa, Acrp30g-2 was selectively identified by antibody directed toward globular head of Acrp30 (FIG. 20C, lanes 2,4) but not by antibodies directed toward the collagen tail of Acrp30 (FIG. 20C, lane 6).

It was further tested whether Acrp30g-2 binding to the conformation-dependent AbAcrp30g antibody was also affected by the blood coagulation process. FIG. 20D shows that spiking of Acrp30g-2 in human blood led to detection of the recombinant protein in plasma after binding to conformation dependent AbAcrp30g antibody covalently bound to RS100 protein chip array followed by Seldi analysis (15.9 kDa). In contrast, spiked Acrp30g-2 was no longer detectable when blood coagulation process was allowed to proceed and serum was obtained. It was verified that Acrp30g-2 was indeed detectable when spiking was performed in serum immediately after termination of coagulation (FIG. 20D).

Conclusion

Both recombinant and physiological polypeptides of SEQ ID NO: 3 undergo structural changes during coagulation.

### Example 23

### Effect of Acrp30g on Tumor Implantation and Growth of Non-Small Cell Lung Carcinoma and Breast Carcinoma Cells in Nude Mice

About 1×10⁶ non-small cell lung carcinoma A549 cells or about 1.5×10⁶ human breast carcinoma MDAMB 231 cells, obtained from the American Tissue Culture Company (ATCC, USA), are injected into the flank of nude mice (Taconic Farms, USA). In addition, the nude mice are injected intraperitoneally with 50 μg/kg to 20 mg/kg of Acrp30g-2 5 minutes before and 4 hours after the subcutaneous injection of the carcinoma cells. Optionally, daily Acrp30g-2 injections are continued for an additional 4 to 9 days or for an additional 10 injections given every other day. Tumor size is measured each day from day 0 to day 20 using a Leica microsystem MML B 100S microscope (Germany) interfaced with a Boeckler Instruments Model 3-MR camera (USA) and RZM Biometrics BQ Nova Prime software (USA). A saline solution is used as a negative control and hirudin at 20 mg/kg may be used as a positive control.

### Example 24

### Effect of Acrp30g on Survival of Nude Mice Undergoing Experimental Pulmonary Metastasis of Non-Small Cell Lung Carcinoma Cells Following Tail-Vein Injection

About 1×10⁶ or 5×10⁶ non-small cell lung carcinoma A549 cells are injected into the tail-vein of nude mice. The nude mice are also injected intraperitoneally with 50 μg/kg to 20 mg/kg of Acrp30g-2 5 minutes before and 4 hours after the subcutaneous injection of the carcinoma cells. Acrp30g-2 injections are continued every other day for 10 days. The survival rate is calculated on 120 days. The lung of all animals dead within 120 days is autopsied. A saline solution is used as a negative control and hirudin at 20 mg/kg may be used as a positive control.

### Example 25

### Effect of Acrp30g Treatment on Growth of Tumors in Syngeneic Mice

About 1×10⁶ B16F10 melanoma cells or 1×10⁵ 4T1 breast carcinoma cells, obtained from the ATCC, are injected subcutaneously into C57BL/6 or BALB/C syngeneic mice. In the experiment with B15F10 cells, the mice are injected with 50 μg/kg to 20 mg/kg of Acrp30g-2 5 minutes before B16F10 implantation as well as 5 consecutive days afterward. In the experiment with 4T1 cells, the mice are injected with 50 μg/kg to 20 mg/kg of Acrp30g-2 5 minutes before 4T1 implantation as well as 10 consecutive days afterward. A saline solution is used as a negative control and hirudin at 10 mg/kg may be used as a positive control.

### Example 26

### Effect of Acrp30g Treatment on eNOS−/− Mice

Introduction

Scientific publications state that full-length Acrp30 increases nitric oxide (NO) production by activating the constitutive form of nitric oxide synthase (eNOS). The mouse and human globular forms of Acrp30 have also been shown to enhance NO production. NO is a potent signaling molecule regulating muscle glucose utilization, causing vasodilation and modulating platelet aggregation. On the basis of this information, the beneficial effect on PE in the animal models could be due to an acute increase of NO production. The effect of Acrp30g-2 in the PE model (see Example 9) using eNOS−/− mice was therefore tested.

Methods

Seven week old eNOS−/− mice (Jackson Laboratories) were housed in a regulatory-approved pathogen-free animal facility with a 12 h light-12 h dark cycle.

Results

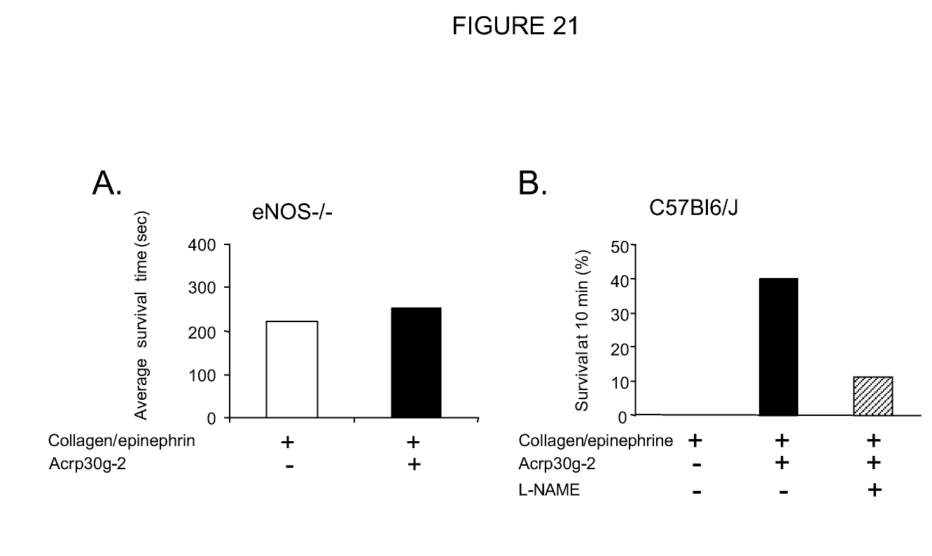

Results in FIG. 21A demonstrates that Acrp30g-2 had no effect on the survival rate after induction of PE in the eNOS−/− mice. Further, in C57BI6/J mice pretreated with the eNOS inhibitor, Nω-Nitro-L-arginine methyl ester hydrochloride (L-NAME), no statistically significant effect of Acrp30g-2 on the survival rate was detected (FIG. 21B).

Conclusion

Taken together, these results indicate that the eNOS enzyme is critical for the anti-thrombotic effect of Acrp30g-2. This conclusion relies on 2 independent methods of investigation: inactivation of eNOS by a small molecule inhibitor as well as genetic inactivation.

### Example 27

### Effect of Acrp30g on a Second Mouse Model for Thrombosis

Introduction

The effect of eNOS activation by Acrp30g was studied using another thrombosis model.

Methods

In this model, the thrombosis occurs in the high pressure arterial compartment. Blood flow was monitored in exposed carotid arteries of anesthetized mice using a Doppler probe. After establishing a baseline level, FeCl₃ was applied as described below. Penetration of the FeCl₃ toxin by diffusion into the arterial wall and lumen causes arterial thrombus formation and a reduced blood flow. After 50% blood flow reduction was achieved, Acrp30g-2 or a saline solution was administered

Arterial thrombosis was induced with FeCl₃ using a procedure adapted from the protocol disclosed in (Wang and Xu, 2005). Mice were anesthetized with sodium pentobarbital (60 mg/kg). After anesthesia, an incision of the skin was made directly on top of the right common carotid artery region. The fascia was then dissected and a segment of the right common carotid was exposed. We measured, using a Transonic® flowprobe (Transonic® Systems, INC), the blood flow in mice carotid artery. After establishing a baseline level, filter paper soaked in 3.75% FeCl₃ solution was applied downstream of the flowprobe and maintained throughout the entire experiment. After 50% of blood flow was achieved, a vehicle (0.9% NaCl) or Acrp30g-2 (400 μg/kg) was injected IP. In a group of mice also treated with Nω-Nitro-L-arginine methyl ester hydrochloride (L-NAME, Sigma), L-NAME (100 mg/kg) was injected IP 1 h before induction of arterial thrombosis with FeCl₃.

Results

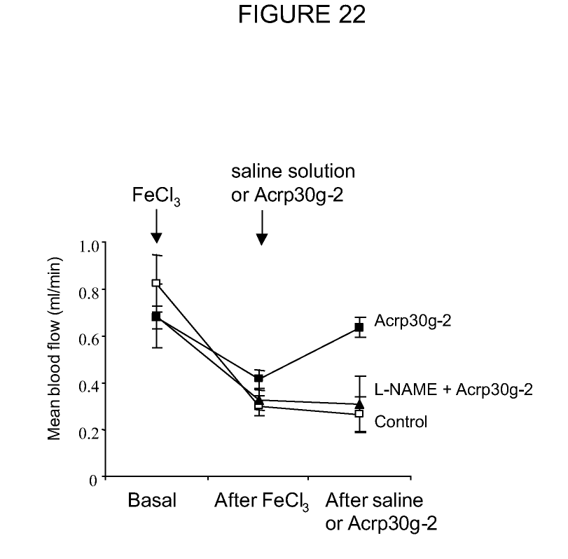

Treatment of animals with Acrp30g-2 restored arterial blood flow to practically baseline levels within 10 min (FIG. 22, closed squares) as compared to controls (FIG. 22, open squares), which showed 50% blood flow reduction. Restoration of blood flow occurred despite the fact that FeCl₃ remained in contact with the carotid artery throughout the experiment. In this model of arterial thrombosis, the anti-thrombotic effect of Acrp30g-2 was suppressed by L-NAME, consistent with the notion that NO production is responsible for restoration of blood flow.

Conclusion

Acrp30g-2 is a therapeutic with potent venous and arterial anti-thrombotic activities. These effects are directly related to the acute release of eNOS-derived NO.

### Example 28

### Effect of Acrp30g on In Vitro Haemostasis

To test the effect of Acrp30g-2 on collagen and epinephrine-induced platelet aggregation, a micro-method allowing monitoring of platelet aggregation on small sample volumes was performed as described in (Walkowiak et al., 1997).

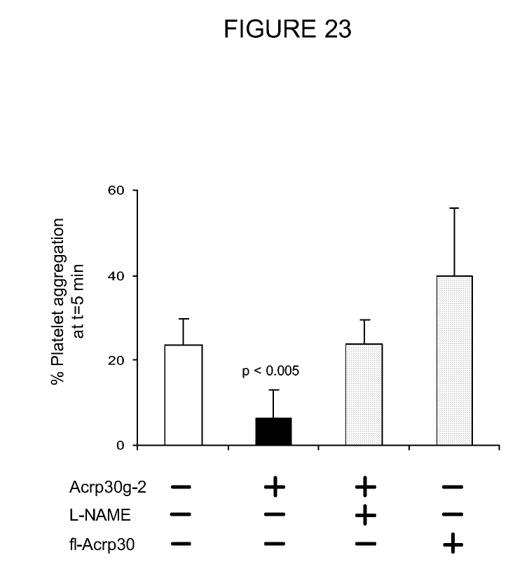

The effect of Acrp30g-2 on platelet aggregation using human PRP was tested at the concentration of 400 ng/ml. Acrp30g-2, but not full-length Acrp30, decreased human platelet aggregation (FIG. 23). The effect was inhibited by eNOS inhibitor L-NAME. The effective therapeutic dose in these in vitro assays was within the range achieved after in vivo injections that were found protective for both arterial and venous thrombosis.

### Example 29

### Acrp30g-2-Induced NO Production in the ECV 304 Cell Line

Introduction

Thrombus formation involves abnormal molecular cross-talk between damaged endothelium and circulating platelets. After demonstrating a direct effect of Acrp30g-2 on platelet aggregation through stimulation of NO production, the effect of Acrp30g-2 on human endothelial cells was investigated. This was carried out by assessing the No production by measuring the nitrate and nitrite levels in the incubation medium. Acrp30g-2-induced NO production was measured in the ECV 304 cell line. ECV 304 cells were incubated 5 min at 37° C. in the presence of increasing doses of Acrp30g-2 or equivalent molar concentrations of full-length Acrp30.

Methods

Cells were plated on Day 0 and used at Day 2 with a confluence of 80-90%. Cells were incubated in pre-warmed DMEM without phenol red in the presence or absence of Acrp30g-2 or full-length Acrp30. In some experiments, cells were pre-incubated with L-NAME before addition of Acrp30g-2, as described in the legend of FIG. 25. After the incubation time, cells were placed on ice, the media recovered and immediately assayed for nitrate and nitrite content using a commercial kit following the manufacturer's instructions (Cayman Chemical).

Results

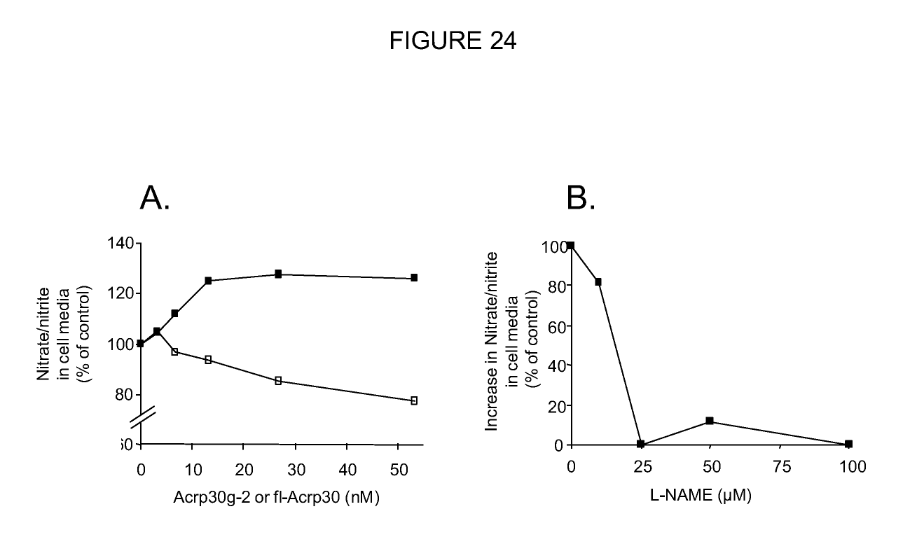

FIG. 24A shows that NO production, measured as the formation of nitrate and nitrite in the media, significantly increased in a dose-dependent manner in cells incubated in the presence of Acrp30g-2. In contrast to this, full-length Acrp30 did not increase NO formation. Full-length Acrp30 even induced a small decrease in NO formation. NO formation stimulated by Acrp30g-2 was inhibited by L-NAME (FIG. 24B). Maximal inhibition was achieved with 25 μM L-NAME.

### REFERENCES

1. Altschul, S. F., Gish, W., Miller, W., Myers, E. W., and Lipman, D. J. (1990). Basic local alignment search tool. J. Mol. Biol. 215, 403-410.
2. Altschul, S. F., Madden, T. L., Schaffer, A. A., Zhang, J., Zhang, Z., Miller, W., and Lipman, D. J. (1997). Gapped BLAST and PSI-BLAST: a new generation of protein database search programs. Nucleic Acids Res. 25, 3389-3402.
3. Angelillo-Scherrer, A., de Frutos, P., Aparicio, C., Melis, E., Savi, P., Lupu, F., Arnout, J., Dewerchin, M., Hoylaerts, M., Herbert, J., Collen, D., Dahlback, B., and Carmeliet, P. (2001). Deficiency or inhibition of Gas6 causes platelet dysfunction and protects mice against thrombosis. Nat. Med. 7, 215-221.
4. Arita, Y., Kihara, S., Ouchi, N., Takahashi, M., Maeda, K., Miyagawa, J., Hotta, K., Shimomura, I., Nakamura, T., Miyaoka, K., Kuriyama, H., Nishida, M., Yamashita, S., Okubo, K., Matsubara, K., Muraguchi, M., Ohmoto, Y., Funahashi, T., and Matsuzawa, Y. (1999). Paradoxical decrease of an adipose-specific protein, adiponectin, in obesity. Biochem. Biophys. Res. Commun. 257, 79-83.

5. ATC (1994). Collaborative overview of randomised trials of antiplatelet therapy—I: Prevention of death, myocardial infarction, and stroke by prolonged antiplatelet therapy in various categories of patients. Antiplatelet Trialists' Collaboration. BMJ 308, 81-106.
6. Berkarda, B., Arda, O., Tasyurekli, M., and Derman, U. (1992). Mitochondria-lytic action of warfarin in lymphocytes. Int. J. Clin. Pharmacol. Ther. Toxicol. 30, 277-279.
7. Berkarda, B., Marrack, P., Kappler, J. W., and Bakemeier, R. F. (1978). Effects of warfarin administration on the immune response of mice. Arzneimittelforschung. 28, 1407-1410.
8. Bhatt, D. L. and Topol, E. J. (2003). Scientific and therapeutic advances in antiplatelet therapy. Nat. Rev. Drug Discov. 2, 15-28.
9. Bihain et al. OBG3 globular head and uses thereof for decreasing body mass [WO 01/51645] 19-07-2001
10. Brutlag, D. L., Dautricourt, J. P., Maulik, S., and Relph, J. (1990). Improved sensitivity of biological sequence database searches. Comput. Appl. Biosci. 6, 237-245.
11. Clark, K. L., Goultier, A., Drew, G. M., Clark, B., and Mitchell, J. N. Adiponectin for treating cardiac disease. [WO 04/10578]. 9-12-2004.
12. Ellington, A. D. and Szostak, J. W. (1990). In vitro selection of RNA molecules that bind specific ligands. Nature 346, 818-822.
13. Fruebis, J., Tsao, T. S., Javorschi, S., Ebbets-Reed, D., Erickson, M. R., Yen, F. T., Bihain, B. E., and Lodish, H. F. (2001). Proteolytic cleavage product of 30-kDa adipocyte complement-related protein increases fatty acid oxidation in muscle and causes weight loss in mice. Proc. Natl. Acad. Sci. U.S. A 98, 2005-2010.
14. Gonnet, G. H., Cohen, M. A., and Benner, S. A. (1992). Exhaustive matching of the entire protein sequence database. Science 256, 1443-1445.
15. Grantham, R. (1974). Amino acid difference formula to help explain protein evolution. Science 185, 862-864.
16. Haines, S. T. (2004). Update on the prevention of venous thromboembolism. Am. J. Health Syst. Pharm. 61, S5-11.
17. Hara, K., Boutin, P., Mori, Y., Tobe, K., Dina, C., Yasuda, K., Yamauchi, T., Otabe, S., Okada, T., Eto, K., Kadowaki, H., Hagura, R., Akanuma, Y., Yazaki, Y., Nagai, R., Taniyama, M., Matsubara, K., Yoda, M., Nakano, Y., Tomita, M., Kimura, S., Ito, C., Froguel, P., and Kadowaki, T. (2002). Genetic variation in the gene encoding adiponectin is associated with an increased risk of type 2 diabetes in the Japanese population. Diabetes 51, 536-540.
18. Hawkins, D. (2004). Economic considerations in the prevention and treatment of venous thromboembolism. Am. J. Health Syst. Pharm. 61, S18-S21.
19. Henikoff, S, and Henikoff, J. G. (1993). Performance evaluation of amino acid substitution matrices. Proteins 17, 49-61.
20. Higgins, D. G., Thompson, J. D., and Gibson, T. J. (1996). Using CLUSTAL for multiple sequence alignments. Methods Enzymol. 266, 383-402.
21. Hu, E., Liang, P., and Spiegelman, B. M. (1996). AdipoQ is a novel adipose-specific gene dysregulated in obesity. J. Biol. Chem. 271, 10697-10703.
22. Hu, L., Lee, M., Campbell, W., Perez-Soler, R., and Karpatkin, S. (2004). Role of endogenous thrombin in tumor implantation, seeding, and spontaneous metastasis. Blood 104, 2746-2751.
23. Hug, C., Wang, J., Ahmad, N. S., Bogan, J. S., Tsao, T. S., and Lodish, H. F. (2004). T-cadherin is a receptor for hexameric and high-molecular-weight forms of Acrp30/adiponectin. Proc. Natl. Acad. Sci. U.S. A 101, 10308-10313.
24. Jackson, S. P. and Schoenwaelder, S. M. (2003). Antiplatelet therapy: in search of the ‘magic bullet’. Nat. Rev. Drug Discov. 2, 775-789.
25. Karlin, S, and Altschul, S. F. (1990). Methods for assessing the statistical significance of molecular sequence features by using general scoring schemes. Proc. Natl. Acad. Sci. U.S. A 87, 2264-2268.
26. Kondo, H., Shimomura, I., Matsukawa, Y., Kumada, M., Takahashi, M., Matsuda, M., Ouchi, N., Kihara, S., Kawamoto, T., Sumitsuji, S., Funahashi, T., and Matsuzawa, Y. (2002). Association of adiponectin mutation with type 2 diabetes: a candidate gene for the insulin resistance syndrome. Diabetes 51, 2325-2328.
27. Lindsay, R. S., Funahashi, T., Hanson, R. L., Matsuzawa, Y., Tanaka, S., Tataranni, P. A., Knowler, W. C., and Krakoff, J. (2002). Adiponectin and development of type 2 diabetes in the Pima Indian population. Lancet 360, 57-58.
28. Maeda, K., Okubo, K., Shimomura, I., Funahashi, T., Matsuzawa, Y., and Matsubara, K. (1996). cDNA cloning and expression of a novel adipose specific collagen-like factor, apM1 (AdiPose Most abundant Gene transcript 1). Biochem. Biophys. Res. Commun. 221, 286-289.
29. Maeda, N., Shimomura, I., Kishida, K., Nishizawa, H., Matsuda, M., Nagaretani, H., Furuyama, N., Kondo, H., Takahashi, M., Arita, Y., Komuro, R., Ouchi, N., Kihara, S., Tochino, Y., Okutomi, K., Horie, M., Takeda, S., Aoyama, T., Funahashi, T., and Matsuzawa, Y. (2002). Diet-induced insulin resistance in mice lacking adiponectin/ACRP30. Nat. Med. 8, 731-737.
30. Michaels. (1964). Cancer incidence and mortality in patients having anticoagulant therapy. Lancet 22, 832-835.
31. Nadar, S. and Lip, G. Y. (2004). Platelet activation in the hypertensive disorders of pregnancy. Expert. Opin. Investig. Drugs 13, 523-529.
32. Nutescu, E. A. (2004). Strategies for cost-effective prevention and treatment of venous thromboembolism. Introduction. Am. J. Health Syst. Pharm. 61, S3-S4.
33. Panidis, D., Kourtis, A., Farmakiotis, D., Mouslech, T., Rousso, D., and Koliakos, G. (2003). Serum adiponectin levels in women with polycystic ovary syndrome. Hum. Reprod. 18, 1790-1796.
34. Pearson, W. R. and Lipman, D. J. (1988). Improved tools for biological sequence comparison. Proc. Natl. Acad. Sci. U.S. A 85, 2444-2448.
35. Petridou, E., Mantzoros, C., Dessypris, N., Koukoulomatis, P., Addy, C., Voulgaris, Z., Chrousos, G., and Trichopoulos, D. (2003). Plasma adiponectin concentrations in relation to endometrial cancer: a case-control study in Greece. J. Clin. Endocrinol. Metab 88, 993-997.
36. Ramsay, J. E., Jamieson, N., Greer, I. A., and Sattar, N. (2003). Paradoxical elevation in adiponectin concentrations in women with preeclampsia. Hypertension 42, 891-894.
37. Roth, J. L., Mobarhan, S., and Clohisy, M. (2002). The Metabolic Syndrome: where are we and where do we go? Nutr. Rev. 60, 335-337.
38. Savage, B., Cattaneo, M., and Ruggeri, Z. M. (2001). Mechanisms of platelet aggregation. Curr. Opin. Hematol. 8, 270-276.
39. Scherer, P. E., Williams, S., Fogliano, M., Baldini, G., and Lodish, H. F. (1995). A novel serum protein similar to Clq, produced exclusively in adipocytes. J. Biol. Chem. 270, 26746-26749.
40. Thompson, J. D., Higgins, D. G., and Gibson, T. J. (1994). CLUSTAL W: improving the sensitivity of progressive multiple sequence alignment through sequence weighting, position-specific gap penalties and weight matrix choice. Nucleic Acids Res. 22, 4673-4680.
41. Tran, T. and Stewart, A. G. (2003). Protease-activated receptor (PAR)-independent growth and pro-inflammatory actions of thrombin on human cultured airway smooth muscle. Br. J. Pharmacol. 138, 865-875.
42. Tsao, T. S., Murrey, H. E., Hug, C., Lee, D. H., and Lodish, H. F. (2002). Oligomerization state-dependent activation of NF-kappa B signaling pathway by adipocyte complement-related protein of 30 kDa (Acrp30). J. Biol. Chem. 277, 29359-29362.
43. Tsao, T. S., Tomas, E., Murrey, H. E., Hug, C., Lee, D. H., Ruderman, N. B., Heuser, J. E., and Lodish, H. F. (2003). Role of disulfide bonds in Acrp30/adiponectin structure and signaling specificity. Different oligomers activate different signal transduction pathways. J. Biol. Chem. 278, 50810-50817.
44. Walkowiak, B., Kesy, A., and Michalec, L. (1997). Microplate reader—a convenient tool in studies of blood coagulation. Thromb. Res. 87, 95-103.
45. Wang, X. and Xu, L. (2005). An optimized murine model of ferric chloride-induced arterial thrombosis for thrombosis research. Thromb. Res. 115, 95-100.
46. Xu, A., Wang, Y., Keshaw, H., Xu, L. Y., Lam, K. S., and Cooper, G. J. (2003). The fat-derived hormone adiponectin alleviates alcoholic and nonalcoholic fatty liver diseases in mice. J. Clin. Invest 112, 91-100.
47. Yamauchi, T., Kamon, J., Ito, Y., Tsuchida, A., Yokomizo, T., Kita, S., Sugiyama, T., Miyagishi, M., Hara, K., Tsunoda, M., Murakami, K., Ohteki, T., Uchida, S., Takekawa, S., Waki, H., Tsuno, N. H., Shibata, Y., Terauchi, Y., Froguel, P., Tobe, K., Koyasu, S., Taira, K., Kitamura, T., Shimizu, T., Nagai, R., and Kadowaki, T. (2003). Cloning of adiponectin receptors that mediate antidiabetic metabolic effects. Nature 423, 762-769.
48. Yamauchi, T., Kamon, J., Waki, H., Terauchi, Y., Kubota, N., Hara, K., Mori, Y., Ide, T., Murakami, K., Tsuboyama-Kasaoka, N., Ezaki, O., Akanuma, Y., Gavrilova, O., Vinson, C., Reitman, M. L., Kagechika, H., Shudo, K., Yoda, M., Nakano, Y., Tobe, K., Nagai, R., Kimura, S., Tomita, M., Froguel, P., and Kadowaki, T. (2001). The fat-derived hormone adiponectin reverses insulin resistance associated with both lipoatrophy and obesity. Nat. Med. 7, 941-946.
49. Yokota, T., Oritani, K., Takahashi, I., Ishikawa, J., Matsuyama, A., Ouchi, N., Kihara, S., Funahashi, T., Tenner, A. J., Tomiyama, Y., and Matsuzawa, Y. (2000). Adiponectin, a new member of the family of soluble defense collagens, negatively regulates the growth of myelomonocytic progenitors and the functions of macrophages. Blood 96, 1723-1732.
50. Zacharski, L. R., Costantini, V., Wojtukiewicz, M. Z., Memoli, V. A., and Kudryk, B. J. (1990). Anticoagulants as cancer therapy. Semin. Oncol. 17, 217-227.
51. Zoccali, C., Mallamaci, F., Panuccio, V., Tripepi, G., Cutrupi, S., Parlongo, S., Catalano, F., Tanaka, S., Ouchi, N., Kihara, S., Funahashi, T., and Matsuzawa, Y. (2003). Adiponectin is markedly increased in patients with nephrotic syndrome and is related to metabolic risk factors. Kidney Int. Suppl S98-102.

1-17. (canceled)

18. A method of treating tumor seeding, metastasis or tumor implantation in a subject comprising the administration of a composition comprising an Acrp30g polypeptide or of an agonist thereof to said subject, wherein said Acrp30g polypeptide is selected from:
a) a polypeptide comprising amino acids 114 to 244 of SEQ ID NO: 1;
b) a polypeptide comprising SEQ ID NO: 2;
c) a polypeptide comprising SEQ ID NO: 3;
d) a polypeptide comprising amino acids 106 to 244 of SEQ ID NO: 1; or
e) a polypeptide comprising amino acids 79 to 244 of SEQ ID NO: 1;
and wherein said Acrp30g polypeptide does not comprise amino acids 1 to 70 of SEQ ID NO: 1; and
said Acrp30g polypeptide has an anti-coagulant and/or an anti-aggregant activity on platelets.

19. The method according to claim 18, wherein said Acrp30g polypeptide is PEGylated.

20. The method according to claim 18, wherein said Acrp30g polypeptide is a fused protein comprising a carrier molecule.

21. The method according to claim 20, wherein said Acrp30g polypeptide is a fused protein comprising an immunoglobulin (Ig) domain.

22. The method according to claim 18, wherein an anti-coagulant and/or anti-aggregant agent different from said Acrp30g polypeptide is simultaneously, sequentially, or separately administered.

23. The method according to claim 18, wherein a fibrinolytic agent is simultaneously, sequentially, or separately administered.

24. The method according to claim 18, wherein a drug for the treatment of cancer is simultaneously, sequentially, or separately administered.

25. A method of treating deep vein thrombosis (DVT), pulmonary embolism (PE), chronic venous insufficiency (CVI), thrombophlebitis or postphlebitic syndrome comprising the administration of a composition comprising an Acrp30g polypeptide or of an agonist thereof to said subject, wherein said Acrp30g polypeptide is selected from:
a) a polypeptide comprising amino acids 114 to 244 of SEQ ID NO: 1;
b) a polypeptide comprising SEQ ID NO: 2;
c) a polypeptide comprising SEQ ID NO: 3;
d) a polypeptide comprising amino acids 106 to 244 of SEQ ID NO: 1; or
e) a polypeptide comprising amino acids 79 to 244 of SEQ ID NO: 1;
and wherein said Acrp30g polypeptide does not comprise amino acids 1 to 70 of SEQ ID NO: 1; and
said Acrp30g polypeptide has an anti-coagulant and/or an anti-aggregant activity on platelets.

26. The method according to claim 25, wherein said Acrp30g polypeptide is PEGylated.

27. The method according to claim 25, wherein said Acrp30g polypeptide is a fused protein comprising a carrier molecule.

28. The method according to claim 27, wherein said Acrp30 polypeptide is a fused protein comprising an immunoglobulin (Ig) domain.

29. The method according to claim 25, wherein an anti-coagulant and/or anti-aggregant agent different from said Acrp30g polypeptide is simultaneously, sequentially, or separately administered.

30. The method according to claim 25, wherein a fibrinolytic agent is simultaneously, sequentially, or separately administered.

31. The method according to claim 25, wherein a drug for the treatment of cancer is simultaneously, sequentially, or separately administered.

32. A method of treating a hypertensive disorder of the pregnancy comprising the administration of a composition comprising an Acrp30g polypeptide or of an agonist thereof to said subject, wherein said Acrp30g polypeptide is selected from:
a) a polypeptide comprising amino acids 114 to 244 of SEQ ID NO: 1;
b) a polypeptide comprising SEQ ID NO: 2;
c) a polypeptide comprising SEQ ID NO: 3;
d) a polypeptide comprising amino acids 106 to 244 of SEQ ID NO: 1; or
e) a polypeptide comprising amino acids 79 to 244 of SEQ ID NO: 1;
and wherein said Acrp30g polypeptide does not comprise amino acids 1 to 70 of SEQ ID NO: 1; and
said Acrp30g polypeptide has an anti-coagulant and/or an anti-aggregant activity on platelets.

33. A method of treating coronary arterial thrombosis, ischemic stroke, intermittent claudication or atrial fibrillation comprising the administration of a composition comprising an Acrp30g polypeptide or of an agonist thereof to said subject, wherein said Acrp30g polypeptide is selected from:
a) a polypeptide comprising amino acids 114 to 244 of SEQ ID NO: 1;
b) a polypeptide comprising SEQ ID NO: 2;
c) a polypeptide comprising SEQ ID NO: 3;
d) a polypeptide comprising amino acids 106 to 244 of SEQ ID NO: 1; or
e) a polypeptide comprising amino acids 79 to 244 of SEQ ID NO: 1;
and wherein said Acrp30g polypeptide does not comprise amino acids 1 to 70 of SEQ ID NO: 1; and
said Acrp30g polypeptide has an anti-coagulant and/or an anti-aggregant activity on platelets.

34. A method of treating arterio-veinous shunts, disseminated intravascular coagulation and tlhrombophilia comprising the administration of a composition comprising an Acrp30g polypeptide or of an agonist thereof to said subject, wherein said Acrp30g polypeptide is selected from:
a) a polypeptide comprising amino acids 114 to 244 of SEQ ID NO: 1;
b) a polypeptide comprising SEQ ID NO: 2;
c) a polypeptide comprising SEQ ID NO: 3;
d) a polypeptide comprising amino acids 106 to 244 of SEQ ID NO: 1; or
e) a polypeptide comprising amino acids 79 to 244 of SEQ ID NO: 1;
and wherein said Acrp30g polypeptide does not comprise amino acids 1 to 70 of SEQ ID NO: 1; and
said Acrp30g polypeptide has an anti-coagulant and/or an anti-aggregant activity on platelets.

35. A method of treating melanomas, leukemias, lymphomas, myelomas or carcinomas comprising the administration of a composition comprising an Acrp30g polypeptide or of an agonist thereof to said subject, wherein said Acrp30g polypeptide is selected from:
a) a polypeptide comprising amino acids 114 to 244 of SEQ ID NO: 1;
b) a polypeptide comprising SEQ ID NO: 2;
c) a polypeptide comprising SEQ ID NO: 3;
d) a polypeptide comprising amino acids 106 to 244 of SEQ ID NO: 1; or
e) a polypeptide comprising amino acids 79 to 244 of SEQ ID NO: 1;
and wherein said Acrp30g polypeptide does not comprise amino acids 1 to 70 of SEQ ID NO: 1; and
said Acrp30g polypeptide has an anti-coagulant and/or an anti-aggregant activity on platelets.


SEQUENCE LISTING (3 sequences)

SEQ ID NO: 1 - protein, 244 aa, Homo sapiens
  SIGNAL (1)..(18)
MLLLGAVLLLLALPGHDQETTTQGPGVLLPLPKGACTGWMAGIPGHPGHNGAPGRDGRDGTPGEKGEKGDPGLIGPKGDIGETGVPGAEGPRGFPGIQGRKGEPGEGAYVYRSAFSVGLETYVTIPNMPIRFTKIFYNQQNHYDGSTGKFHCNIPGLYYFAYHITVYMKDVKVSLFKKDKAMLFTYDQYQENNVDQASGSVLLHLEVGDQVWLQVYGEGERNGLYADNDNDSTFTGFLLYHDTN

SEQ ID NO: 2 - protein, 136 aa, Homo sapiens
MVYRSAFSVGLETYVTIPNMPIRFTKIFYNQQNHYDGSTGKFHCNIPGLYYFAYHITVYMKDVKVSLFKKDKAMLFTYDQYQENNVDQASGSVLLHLEVGDQVWLQVYGEGERNGLYADNDNDSTFTGFLLYHDTN

SEQ ID NO: 3 - protein, 137 aa, Homo sapiens
AYVYRSAFSVGLETYVTIPNMPIRFTKIFYNQQNHYDGSTGKFHCNIPGLYYFAYHITVYMKDVKVSLFKKDKAMLFTYDQYQENNVDQASGSVLLHLEVGDQVWLQVYGEGERNGLYADNDNDSTFTGFLLYHDTN